(19) United States
(12) Patent Application Publication
(10) Pub. No.: US 2022/0395293 A1
(43) Pub. Date: Dec. 15, 2022

(54) TRANSVAGINAL ACCESS DEVICES AND METHODS OF USE

(71) Applicant: Atropos Limited, Bray (IE)

(72) Inventors: Stephen WILLIAMS, Blackrock (IE); Frank BONADIO, Bray (IE); Kieran GIBBONS, Ramelton (IE); Shane MacNally, Delgany (IE); Ronan McManus, Bray (IE); Olwen COUGHLAN, Dublin 18 (IE); Adam GOLDRICK, Ballinamore (IE); Sani MACNALLY, Delgany (IE); Colm Flynn, Enfield (IE); Lucy Dolores HALPIN, Dublin (IE)

(73) Assignee: Atropos Limited, Bray (IE)

(21) Appl. No.: 17/838,864

(22) Filed: Jun. 13, 2022

Related U.S. Application Data
(60) Provisional application No. 63/297,103, filed on Jan. 6, 2022.
(60) Provisional application No. 63/257,220, filed on Oct. 19, 2021.
(60) Provisional application No. 63/209,831, filed on Jun. 11, 2021.

(51) Int. Cl. A61B 17/34

(52) U.S. Cl. CPC A61B 17/3423; A61B 17/3462; A61B 2017/3445; A61B 2017/345; A61B 2017/3464

(57) ABSTRACT

A medical device to protect a body opening, such as a vagina. The medical device may include a lumen and anchoring structure(s) for securing the device in the body opening. In some embodiments, the medical device includes a sleeve, a proximal ring attached to a proximal end of the sleeve, and a distal ring attached to a distal end of the sleeve.

### CROSS-REFERENCE TO RELATED APPLICATIONS

This application claims the benefit of priority of U.S. Provisional Patent Application No. 63/209,831, filed Jun. 11, 2021, U.S. Provisional Patent Application No. 63/257,220, filed Oct. 19, 2021, and U.S. Provisional Patent Application No. 63/297,103, filed Jan. 6, 2022, the entirety of all of which are incorporated herein by reference.

### TECHNICAL FIELD

The present disclosure relates generally to medical systems and devices for accessing a body cavity, and more particularly, to methods and devices for transvaginal access during a medical procedure.

### BACKGROUND

Procedures occurring in a body cavity accessed via an opening in a body, e.g., a natural orifice such as a transvaginal opening or via an incision, may require a device to assist in accessing the body cavity. In some instances, a fluid (e.g., a gas or other fluid) may be supplied to insufflate the body cavity to aid in performing the medical procedure. Absent a fluid seal, the fluid may escape via the body opening. Medical procedures may also require medical instruments that may be too large to fit through the body opening. For example, a diameter of the body opening may be too small to provide sufficient access to medical instruments to perform the medical procedure. Additionally, the size and/or the shape of the body opening may decrease the ability of the user to properly access a target site within the body cavity. This disclosure may address one or more of these problems or other problems in the art. The scope of the disclosure, however, is defined by the attached claims and not the ability to address a specific problem.

### SUMMARY OF THE DISCLOSURE

According to an aspect, a medical device configured to protect a body opening, the medical device including: a sleeve having a proximal end and a distal end; a proximal ring attached to the proximal end of the sleeve; and a distal ring attached to the distal end of the sleeve.

In another embodiment, a medical device configured to protect a body opening, the medical device including: a sleeve having a proximal end and a distal end; a proximal ring attached to the proximal end of the sleeve; a distal ring attached to the distal end of the sleeve; and one or more valves at the proximal end of the sleeve.

In another embodiment, a method of using a medical device configured to protect a body to perform a surgical procedure, the method including: positioning the medical device within a body cavity, the medical device including: a sleeve having a proximal end, a distal end, an outer wall, and an inner wall; a proximal ring attached to the proximal end of the sleeve; a distal ring attached to the distal end of the sleeve; and wherein the proximal ring is positioned outside the body cavity and the distal ring is positioned inside the body cavity; expanding the outer wall of the sleeve so that a cavity wall of the body cavity retracts; inserting one or more surgical tools through the medical device and into the body cavity; and performing the surgical procedure using at least the one or more surgical tools.

In another embodiment, a medical access device for use in a body opening, including: a lumen having a proximal end and a distal end; a movable member configured to move between an insertion position and a radially expanded securing position; and a stabilizing element configured to increase the rigidity of the medical access device.

In another embodiment, a medical device including: a proximal ring defining an opening; an inflatable sleeve extending distally from the proximal ring and defining a lumen, the lumen connected to the opening; and at least one tab extending from the proximal ring into the lumen.

In another embodiment, a medical device including: a first tab and a second tab; a distal ring connected to a distal end of each of the first tab and the second tab; and an inflatable sleeve connected to the first tab and the second tab.

In another embodiment, a medical device, including: a first crossbar and a second crossbar, wherein the first and the second crossbars are parallel; a first movable member having a first opening and a second opening, the first opening receiving a portion of the first crossbar and the second opening receiving a portion of the second crossbar; a second movable member having a third opening and a fourth opening, the third opening receiving a portion of the first crossbar and the fourth opening receiving a portion of the second crossbar; and an inflatable sleeve extending from the first crossbar and the second crossbar to distal ends of the first movable member and the second movable member.

In another embodiment, a medical device including: an annular member defining a lumen; an inflatable member attached to a radially outer surface of the annular member; and a cap configured to fluidly seal a proximal end of the medical device.

In another embodiment, a medical device including: an annular member defining a lumen, wherein the annular member is configured to expand from a non-expanded configuration, in which a cross-section of the annular member is generally circular, to an expanded configuration, in which the cross-section of the annular member is generally elliptical; and an inflatable member attached to a radially outer surface of a distal end of the annular member.

In another embodiment, a medical device including: an inflatable member defining a central lumen; and a distal ring attached to a distal end of the inflatable member.

In another embodiment, a medical device including: a first tab and a second tab and defining a lumen therebetween; a distal ring connecting a distal end of the first tab to a distal end of the second tab; a sleeve fluidly sealing a proximal end of the lumen; and an inflatable ring connected to a radially outer surface of the first tab and a radially outer surface of the second tab.

In another embodiment, a medical access device for use in a body opening, including: an outer member including: a lumen having a proximal end and a distal end; a flat base; and a pair of side walls connected to the flat base.

In another embodiment, a method of using a medical access device configured to protect a body to perform a surgical procedure, the method including: positioning the medical access device within a body cavity, the medical access device including: an lumen having a proximal end and a distal end; a flat base; a pair of side walls connected to the flat base; inserting one or more medical instruments through the medical access device and into the body cavity; and performing the surgical procedure using at least one or more surgical instruments.

### BRIEF DESCRIPTION OF THE DRAWINGS

FIG. 1 is a perspective view of a medical device for accessing the inside of a patient;

FIGS. 2A-2D are perspective views of the medical device of FIG. 1, according to an example;

FIGS. 2E and 2F are cross sectional views of the medical device of FIG. 1, according to an example;

FIGS. 3A-3C are perspective views of the medical device of FIG. 1 after the device is inflated, according to an example;

FIGS. 4A and 4B are views of the medical device of FIG. 1 inserted into a vagina, according to an example;

FIGS. 5A and 5B are views of a medical device, according to another example;

FIG. 6A is a perspective view of the medical device of FIG. 1, according to an example;

FIGS. 6B and 6C are cross sectional views of the inflatable medical device of FIG. 6A, according to an example;

FIG. 6D is a view of the medical device of FIG. 6A positioned in a body opening, according to an example;

FIG. 7 is a side view of a distal ring, according to an example;

FIGS. 8A-8E are cross-sectional views of a medical device including an inverted sleeve, according to an example;

FIGS. 9A-9E are cross-sectional views of the distal ring of FIG. 7 and the medical device of FIG. 8A;

FIGS. 10A-10F are cross-sectional views of a medical device, according to another example;

FIGS. 11A and 11B are views of the medical device of FIG. 1OF positioned in a body opening, according to an example;

FIGS. 12A is a cross-sectional view of medical instruments being inserted through the medical device of FIG. 11A, according to an example;

FIG. 12B is a cross-sectional view of an embodiment of a medical device, according to an example;

FIGS. 13A-13C are cross-sectional views of a medical device, according to an example;

FIGS. 14A-14D are side views of a medical device including an inflatable ring, according to an example;

FIG. 15A is a side view of a medical device, according to an example;

FIGS. 15B and 15C illustrate the medical device of FIG. 15A inserted into a body opening;

FIG. 16A is a side view of a medical device, according to an example;

FIGS. 16B and 16C illustrate the medical device of FIG. 16A inserted into a body opening;

FIGS. 17A-17C illustrate a medical device, according to an example;

FIG. 17D illustrates the medical device of FIG. 17A positioned in a body opening, according to an example;

FIGS. 18A and 18B are cross sectional views of a distal end of the medical device of FIGS. 17A-17D, according to an example;

FIG. 19 is a cross-sectional view of an inflatable ribbed distal end of the medical device of FIGS. 17A-17D, according to an example;

FIGS. 20A-20C illustrate a medical device, according to an example;

FIGS. 21A-21D illustrate a medical device according to an example;

FIGS. 22A-22D illustrate a medical device, according to an example;

FIGS. 23A and 23B are views of a medical device positioned in a body opening, according to an example;

FIG. 24A is a perspective view a retractor medical device, according to an example;

FIG. 24B illustrates a medical device positioned in a body opening, according to an example;

FIGS. 25A-25D illustrate a medical device including valve cap with multiple ports, according to an example;

FIGS. 26A and 26B illustrate a container removably coupled to a cap of a medical device, according to an example;

FIGS. 27A-27C illustrate a medical device, according to an example;

FIGS. 28A and 28B illustrate a medical device, according to an example;

FIGS. 29A and 29B illustrate components decoupled from the medical device of FIGS. 28A and 28B, according to an example;

FIGS. 30A and 30B illustrate the medical device of FIGS. 26A and 26B, according to an example;

FIG. 31 illustrates a medical device, according to an example;

FIG. 32 is a cross-sectional view of a cap for an access device, according to an example;

FIGS. 33A and 33B illustrate a cap for a medical device, according to an example; and

FIG. 34 is illustrates a medical device positioned in a body opening, according to an example.

FIGS. 35A-35D illustrate a medical device for use with a body opening, according to an example;

FIGS. 36A-36F illustrate a method of performing a medical procedure using the medical device of FIGS. 35A-35D;

FIGS. 37A-37C illustrate a medical device for use with a body opening, according to another example;

FIGS. 38A and 38B illustrate a medical device for use with a body opening, according to another example;

FIGS. 39A-39C illustrate a method of performing a medical procedure using the medical device of FIGS. 38A and 38B;

FIGS. 40A-40C illustrate a medical device for use with a body opening, according to another example;

FIGS. 41A-41C illustrate a method of performing a medical procedure using the medical device of FIGS. 40A-40C;

FIGS. 42A-42C illustrate a medical device for use with a body opening, according to another example;

FIGS. 43A-43C illustrate a method of performing a medical procedure using the medical device of FIGS. 42A-42C;

FIGS. 44A-44D illustrate a medical device for use with a body opening, according to another example;

FIGS. 45A-45C illustrate a method of performing a medical procedure using the medical device of FIGS. 44A-44D;

FIGS. 46A-46C illustrate a medical device for use with a body opening, according to another example;

FIGS. 47A and 47B illustrate a method of performing a medical procedure using the medical device of FIGS. 46A-46C;

FIGS. 48A-48F illustrate a medical device for use with a body opening, according to another example;

FIGS. 49A-49C illustrate a method of performing a medical procedure using the medical device of FIGS. 48A-48F;

FIGS. 50A-50F illustrate a medical device for use with a body opening, according to another example;

FIGS. 51A-51C illustrate a method of performing a medical procedure using the medical device of FIGS. 50A-50F;

FIGS. 52A-52D illustrate a medical device for use with a body opening, according to another example;

FIGS. 53A-53C illustrate a method of performing a medical procedure using the medical device of FIGS. 52A-52D;

FIGS. 54A-54D illustrate a medical device for use with a body opening, according to another example;

FIGS. 55A-55C illustrate a method of performing a medical procedure using the medical device of FIGS. 54A-54D;

FIGS. 56A-56D illustrate a medical device for use with a body opening, according to another example;

FIGS. 57A-57C illustrate a method of performing a medical procedure using the medical device of FIGS. 56A-56D;

FIGS. 58A-58C illustrate a medical device for use with a body opening, according to another example;

FIGS. 59A and 59B illustrate a medical device for use with a body opening, according to another example;

FIGS. 60A-60D illustrate a medical device for use with a body opening, according to another example;

FIGS. 61A-61C illustrate a method of performing a medical procedure using the medical device of FIGS. 60A-60D;

FIGS. 62A-62C illustrate a medical device for use with a body opening, according to another example;

FIGS. 63A-63C illustrate a medical device for use with a body opening, according to another example;

FIGS. 64A-64C illustrate a method of performing a medical procedure using the medical device of FIGS. 62A-62C;

FIGS. 65A and 65B illustrate a medical device for use with a body opening, according to another example;

FIGS. 66A and 66B illustrate a method of performing a medical procedure using the medical device of FIGS. 65A and 65B;

FIGS. 67A-67C illustrate a medical device for use with a body opening, according to another example;

FIGS. 68A-68C illustrate a medical device for use with a body opening, according to another example;

FIGS. 69A and 69B illustrate a medical device for use with a body opening, according to another example;

FIGS. 70A and 70B illustrate a medical device for use with a body opening, according to another example;

FIGS. 71A and 71B illustrate a medical device for use with a body opening, according to another example;

FIGS. 72A-72E illustrate a medical device for use with a body opening, according to another example;

FIGS. 73A-73C illustrate a method of performing a medical procedure using the medical device of FIGS. 72A-72E;

FIGS. 74A and 74B illustrate a medical device for use with a body opening, according to another example

FIGS. 75A-75D illustrate a medical device for use with a body opening, according to another example;

FIGS. 76A and 76B illustrate a method of performing a medical procedure using the medical device of FIGS. 75A-75D;

FIGS. 77A-77I illustrate a medical device for use with a body opening, according to another example;

FIGS. 78A-78D illustrate a medical device for use with a body opening, according to another example;

FIG. 79 illustrates a method of performing a medical procedure using the medical device of FIGS. 78A-78D; and

FIGS. 80A and 80B illustrate a medical device for use with a body opening, according to another example.

FIGS. 81A-81C illustrate a medical device for use with a body opening, according to another example.

FIGS. 82A-82C illustrate a medical device for use with a body opening, according to another example.

FIGS. 83A-83C illustrate insertion of the medical devices of FIGS. 81A-82C in a body opening

FIGS. 84A-84D are views of a medical device inserted into a body opening, according to an example;

FIGS. 85A-85E are views of certain components of the medical access device of FIGS. 84A-84D, according to an alternative examples;

FIGS. 86A-86B are views of the medical access device of FIG. 85C, according to an example;

FIGS. 87A-87E are views of the medical access device of FIG. 85C, according to alternative examples;

FIG. 88 is a side view of the medical access device of FIGS. 87E in a body opening, according to an example;

FIGS. 89A-89E are views of the medical access devices 85C, according to alternative examples;

FIGS. 90A-90D are views of the medical access device 85C, according to alternative examples;

FIGS. 91A-91D are views of a medical access device for accessing the inside of a patient;

FIGS. 92A is a perspective view of a medical access device for use with a body opening;

FIGS. 92B-92D are cross sectional views of the medical access device of FIG. 92A, according to an example;

FIGS. 93A-93B are views of the medical access device of FIG. 91A, according to an example;

FIGS. 94A-94B are perspective views of a section of the medical device of FIG. 92A, according to an example;

FIGS. 95A-95C are perspective views of a section of the medical device of FIG. 92A, according to an example.

### DETAILED DESCRIPTION

The present disclosure is now described with reference to exemplary medical access devices that may be used in accessing the interior of the body. However, it should be noted that reference to any particular procedure is provided only for convenience and not intended to limit the disclosure. A person of ordinary skill in the art would recognize that the concepts underlying the disclosed devices and application methods may be utilized in any suitable procedure, medical or otherwise. The present disclosure may be understood with reference to the following description and the appended drawings, wherein like elements are referred to with the same reference numerals.

For ease of description, portions/regions/ends of a device and/or its components are referred to as proximal and distal ends/regions. It should be noted that the term “proximal,” is intended to refer to ends/regions closer to a user of the disclosed device (e.g., outside the body of the patient), and the term “distal,” used herein to refer to ends/regions farther away from the user of the disclosed device (e.g., inside the body of the patient). Similarly, extends “distally” indicates that a component extends in a distal direction, and extends “proximally” indicates that a component extends in a proximal direction. Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

The present disclosure relates generally to medical systems and devices for accessing a body cavity, and more particularly, to methods and devices for transvaginal access during a medical procedure.

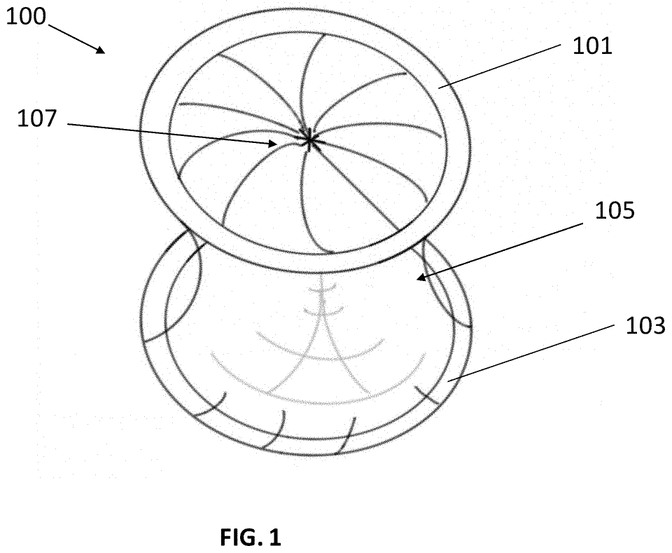

FIG. 1 illustrates a medical device 100 according to an example. Medical device 100 may include a proximal ring 101, a distal ring 103, and a sleeve 105 connecting proximal 101 ring and distal ring 103. Medical device 100 may be a device for accessing a body cavity via an opening in the body, e.g., via an incision or a natural orifice (e.g., a vagina). In an example, medical device 100 may be a transvaginal access device for accessing the vagina. Medical device 100 may be positioned so that the distal ring 103 may be positioned inside the body via the opening and the proximal ring 101 may be positioned outside the body opening, e.g., on an outer surface of the body. Distal ring 103 may be an annular ring, may be flexible, and/or may be formed of, for example, an elastomer. Distal ring 103 may be bendable or otherwise manipulated for insertion into an opening of a body, e.g., a vagina. Distal ring 103 may have sufficient rigidity to position medical device 100 within the body opening during a medical procedure. For example, medical device 100 may remain within in the body opening absent a force applied to medical device 100 sufficient to overcome a holding force of distal ring 103 within the body opening.

Proximal ring 101 may be an annular ring and may be similar to distal ring 103 and may be positioned outside the body opening. Alternatively, proximal ring 101 and distal ring 103 may be of different materials, shapes, and/or dimensions. Proximal ring 101 may have a rigidity sufficient to maintain a generally annular opening. For example, proximal ring 101 may be elastomeric, but may have a rigidity sufficient to provide access to the body opening via an opening in proximal ring 101.

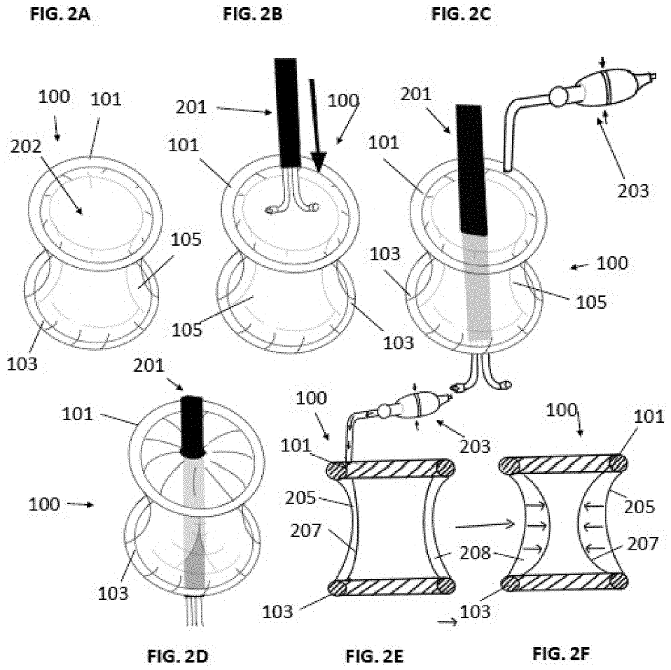

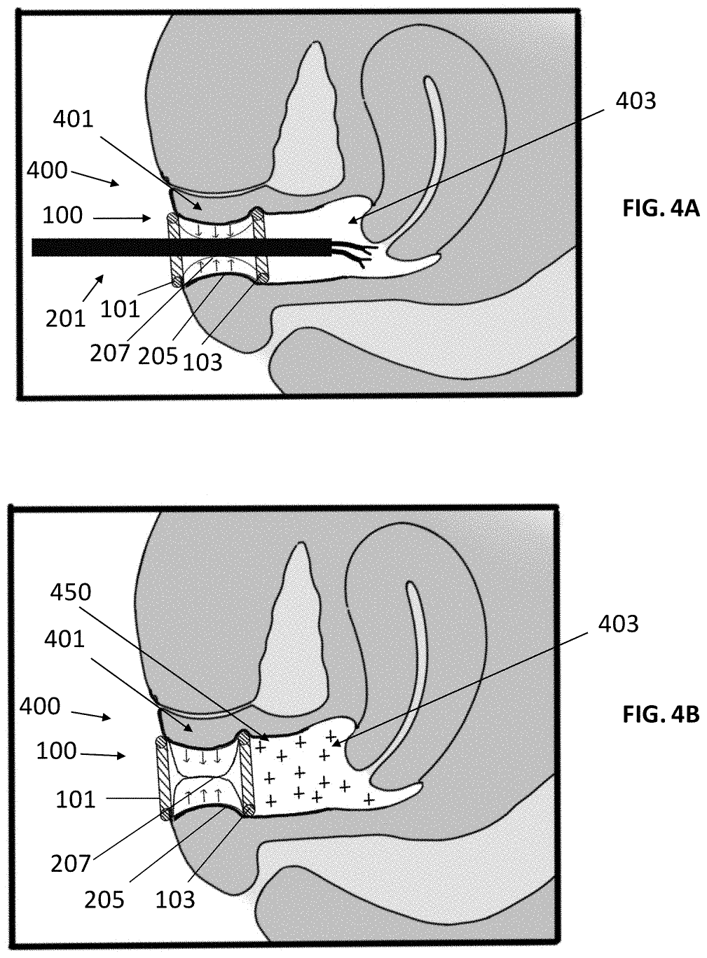

Sleeve 105 may contact tissue in the body opening and may provide access to a body cavity of the patient. Sleeve 105 may provide a radial force sufficient to provide access through the body opening of the patient. Sleeve 105 may include two walls or layers. For example, sleeve 105 may include a radially inner layer and a radially outer layer and may form a sealed cavity (e.g., cavity 403 shown in FIG. 4A-4B) there between. Sleeve 105 may be sealably coupled to the proximal and distal rings 101, 103. Sleeve 105 may define a lumen (e.g., a lumen 202 shown in FIG. 2A) extending from, and connecting, the opening of ring 101 to the opening of ring 103. A radially internal wall of sleeve 105 may be a twisted internal wall and may form an iris or eye configuration. When cavity of sleeve 105 is inflated, e.g., via air or other gas, the radially internal wall may create a fluid seal 107 through the center of the medical device 100. A medical tool (e.g., tool 201 shown in FIG. 2B) may be inserted through the seal 107 of the medical device 100. As the medical tool is inserted through seal 107, the medical tool may push or urge walls of sleeve 105 radially outward. The medical tool may be, for example, a scalpel, grasper, imaging device, cutting device, suction device, thermometer, medical sensor, or other similar medical instruments.

FIGS. 2A-2F illustrate medical device 100. FIG. 2A shows transvaginal medical device 100 having a lumen 202 defined by walls of sleeve 105 and extending between the opening of proximal ring 101 and the opening of distal ring 103. Lumen 202 may provide access to the body cavity during a medical procedure. For example, walls of sleeve 105 may urge tissue surrounding the body opening radially outwards. In this manner, the user may access the body cavity via lumen 202.

FIG. 2B shows a surgical tool 201 (surgical instrument) positioned at or adjacent the opening of proximal ring 101. As shown by arrow B, tool 201 may be moved into lumen 202 and toward the body cavity. As described above, tool 201 may be any surgical instrument or medical device used in a medical procedure, including a scalpel, grasper, imaging device, cutting device, suction device, thermometer, medical sensor, and/or other medical instrument.

FIG. 2C shows an inflation device 203 being used to inflate the cavity of sleeve 105 (e.g. fluid cavity 208 shown in FIG. 2E). The cavity of sleeve 105 may be inflated after tool 201 has been inserted through lumen 202 and into the body cavity. Inflation device 203 may be a manual inflator, e.g., a handheld bulb pump. Inflation device 203 may also be a motorized pump capable of inflating sleeve 105 by moving fluid into sleeve 105 and/or deflating sleeve 105 by removing fluid out of sleeve 105. Inflation device 203 may be removably coupled to the fluid receiving cavity a port. Alternatively, inflation device 203 may be fixed to medical device 100 as a single unit. Inflating the cavity of sleeve 105 may cause the radially inner wall 207 of sleeve 105 to move radially inward and may minimize or close lumen 202, as shown in FIG. 2D. Deflating the cavity of sleeve 105 may cause radially inner wall 207 to move radially outward toward outer wall 205 and may open or maximize the size of lumen 202

FIG. 2D shows tool 201 inserted into medical device 100. Lumen 202 may be closed off such that sleeve 105 contacts an outer surface of tool 201, such that the sleeve 105 is sealed around tool 201. Seal 107 may allow the body cavity to maintain a pressure with the body cavity and/or may prevent fluids or other materials from escaping the body cavity. In one example, the medical tool 201 may be a scope (e.g., a duodenoscope, a ureterscope, a colonoscope, an endoscope, etc.) or similar device. In this instance, one or more medical instruments may be inserted through the scope to perform a medical procedure within the body cavity.

FIGS. 2E and 2F show medical device 100 with inflation device 203 being used to inflate sleeve 105. Sleeve 105 may be inflated by supplying a fluid into cavity 208 formed between inner wall 207 and outer wall 205 of sleeve 105. For example, as fluid is supplied to cavity 208, inner wall 207 may move radially inward toward a central axis of lumen 202 and away from outer wall 205, as indicated by arrows C in FIG. 2F. Sleeve 105 may be deflated by purging or removing the fluid from cavity 208, which may cause inner wall 207 to move radially outward toward outer wall 205 (opposite arrows C in FIG. 2F).

In some examples, sleeve 105 may be a single piece of material that extends from the proximal ring 101, around the distal ring 103, and back to the proximal ring 101. In this manner, sleeve 105 may form inner wall 207 and outer wall 205. For example, sleeve 105 may extend distally from the proximal ring 101 to wrap around the outer surface of distal ring 103 to create outer wall 205. Sleeve 105 may then extend from the inner surface of distal ring 103 to the inner surface of proximal ring 101 to create inner wall 207.

A method of using medical device 100 will now be explained with reference to FIGS. 2A-2F. Medical device 100 may be positioned in a body opening, e.g., an incision in the body or a natural orifice (e.g., a vagina). Distal ring 103 may be inserted into the body opening and advanced toward the body cavity. In some instances, distal ring 103 may be positioned in the body opening. In other instances, distal ring 103 may be positioned in the body cavity and may anchor medical device 100 in the body opening. For example, distal ring 103 may contact tissue defining the body cavity and may anchor medical device within the body opening. Proximal ring 101 may be positioned on an outer surface of the patient.

Cavity 208 may be uninflated during insertion of medical device 100. Once medical device 100 is inserted in the body opening, tool 201 may be inserted into lumen 202 via the opening in proximal ring 101 as shown by arrow B in FIG. 2B. After positioning tool 201 within lumen 202, cavity 208 of sleeve 105 may be inflated via inflation device 203. Inflating cavity 208 may create a fluid seal between sleeve 105 and tool 201, as shown in FIG. 2D. As described above, tool 201 may be a scope and may include one or more lumens through which medical instruments may be inserted and advanced into the body cavity to perform a medical procedure.

Once the medical procedure is complete, cavity 208 may be deflated using inflation device 203 and/or a fluid release valve coupled to cavity 208. Releasing fluid from cavity 208 may break the fluid seal between sleeve 105 and tool 201 and may allow tool 201 to be removed from lumen 202. After tool 201 is removed from lumen 202, medical device 100 may be removed from the body opening. For example, a user may pull on proximal ring 101 with a force sufficient to overcome the anchoring force of distal ring 103. Alternatively, or additionally, the user may use a thumb and one or more fingers to grasp distal ring 103 and pull distal ring 103 through the body opening. It will be understood that a medical instrument may also be used to grasp distal ring 103 to remove medical device 100 from the body opening.

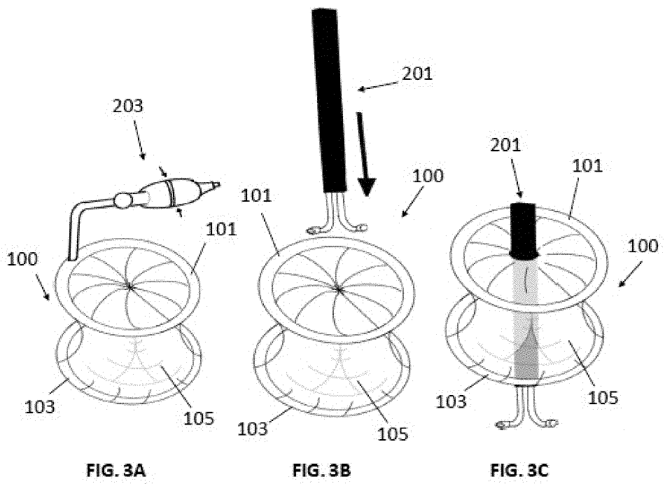

FIGS. 3A-3C illustrate tool 201 being inserted into medical device 100 after sleeve 105 is inflated. In this manner, a fluid seal between a body cavity and the outside of the body may be formed before tool 201 is inserted into the body. When the tool 201 is inserted through lumen 202 of medical device 100, sleeve 105 may seal around tool 201. Sleeve 105 may be a double sleeve with inner wall 207 sealed around the tool 201 and outer wall 205 sealing against the body opening. A cavity (e.g., cavity 208 in FIG. 2E) formed by inner wall 207 and outer wall 205 may be inflatable and may cause sleeve 105 to seal lumen 202 and/or create a seal with tissue surrounding the body opening when the cavity is inflated. Sleeve 105 may have increased rigidity when the cavity is inflated. The cavity formed by sleeve 105 may be inflated after tool 201 is inserted into lumen 202 or, alternatively, before tool 201 is inserted.

Medical device 100 may be inserted into a body opening (e.g., a natural orifice or an incision) to provide access to a body cavity 403, shown in FIGS. 4A and 4B. For example, proximal ring 101 may be positioned outside body cavity 403 and distal ring 103 may be positioned inside body cavity 403. For example, medical device 100 may be inserted into a vagina 400 so that a surgeon may access cavity 403 (e.g., a vaginal cavity) to perform a medical procedure.

FIG. 4A shows medical device 100 inserted within a vagina 400. Medical device 100 may be designed to have one or more sizes and may fit in different sized openings. In some instances, medical device 100 may be larger than vagina 400 and may create a seal against a tissue/vaginal wall 401 surrounding the opening of vagina 400. Medical device 100 may be inflated by inflation device 203 while seated within the vagina 400. Medical device 100 may cause vaginal wall 401 surrounding the opening of vagina 400 to move radially outward (e.g., retract). For example, proximal ring 101 and distal ring 103 may be formed of a material that urges sleeve 105 radially outward (with or without inflation). Inflating sleeve 105 may cause vaginal wall 401 to move further outward in a radial direction. Vaginal wall 401 may be held or urged radially outward by outer wall 205 of sleeve 105. Surgical tool 201 may be extended through an opening in proximal ring 101, sleeve 105, and distal ring 103 to access cavity 403. Inner wall 207 of sleeve 105 may seal around the tool 201 and may prevent fluid or other bodily materials from escaping cavity 403 during the medical procedure.

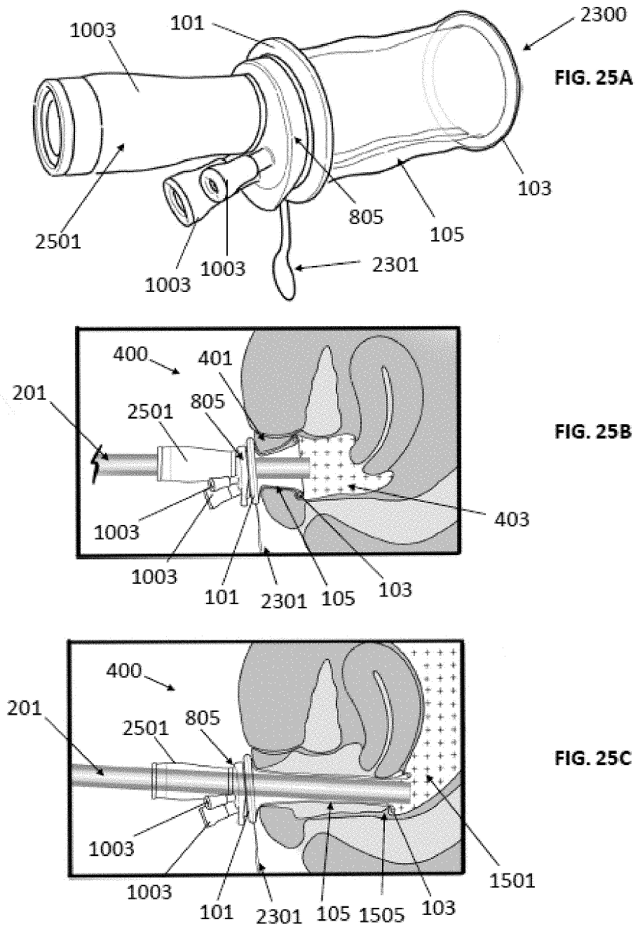

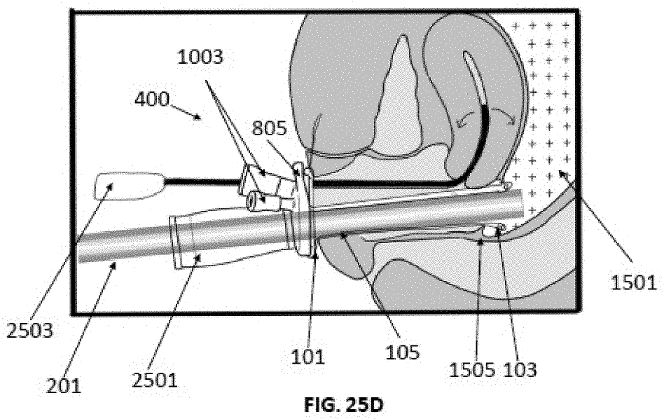

FIG. 4B shows medical device 100 inserted within a vagina 400. The medical device 100 may be capable of providing and maintaining insufflation interior of the opening (e.g., the vagina and/or abdomen) as will be discussed in further detail below. Alternatively, the insufflation (e.g., the flow of insufflation gas) can originate from the peritoneum and be sealed by the medical device 100 (FIG. 4C). Alternatively, the flow of insufflation gas could enter the body opening through an incision in the vaginal wall (see, e.g., FIG. 25D). The vagina 400 cavity 403 may be insufflated to expand cavity 403, which may create additional space within the body for the medical procedure. The medical device 100, while expanded, may be capable of retaining penumo pressure within the cavity 403 as indicated by the + signs within cavity 403 (+ signs may indicate pnemo pressure within a cavity). This may occur when inner wall 207 of sleeve 105 come into contact with either itself or a medical tool 201 (not shown).

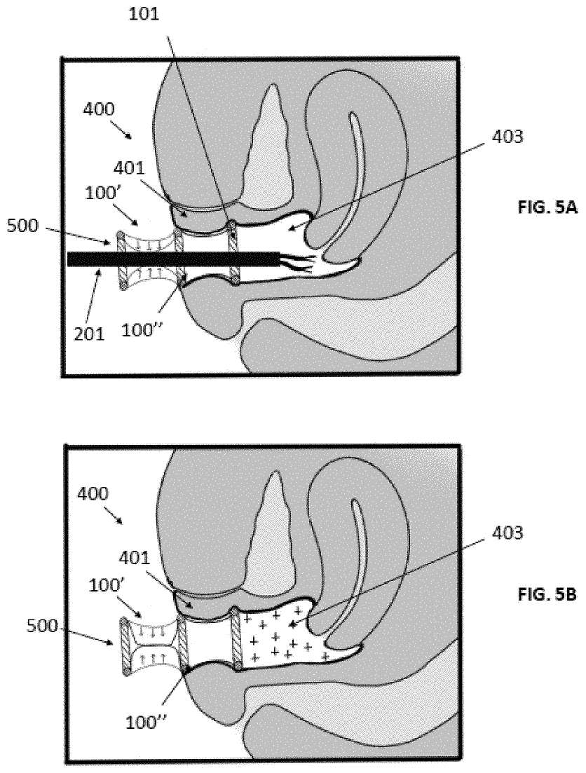

FIG. 5A shows a medical device 500 including a proximal access device 100′ and a distal access device 100″, according to another example. Proximal access device 100′ may be placed external to the opening of cavity 403 and may form an external seal. Distal access device 100″ may be placed inside cavity 403 of vagina 400 and may contact an inner surface of vagina 400. It is understood that distal access device 100″ may include two layers or walls or just a single layer or wall. As shown, the distal access device 100″ is anchored in the cavity 403 of vagina 400 (similar to FIGS. 4A and 4B). In this embodiment, the inflated proximal access device 100′ of medical access device 500 external to the anatomy. In some instances, distal access device 100″ may be inflated. In another embodiment, distal access device 100″ may hold open the vaginal walls of cavity 403 vagina 400. The rigid material of distal access device 100″ may allow for the device to stay in place and hold open the vaginal walls of cavity 403 of vagina 400.

When the sealing proximal device 100′ is located outside of the body, there may more space within the opening of the vagina 400. This may allow for a surgeon to have more space or a better angle to navigate surgical tool 201 during medical procedures. Further, an external seal may provide for a better and/or easier to maintain seal compared to an internal seal. FIG. 5B illustrates the proximal access device 100′ forming a seal completely without instruments (e.g., no surgical tool 201 is depicted) in situ to maintain pressure of the cavity 403. In FIG. 5B, proximal access device 100′, may still form a seal and maintain pressure, even when no surgical tool 201 is located within medical access device 500. FIG

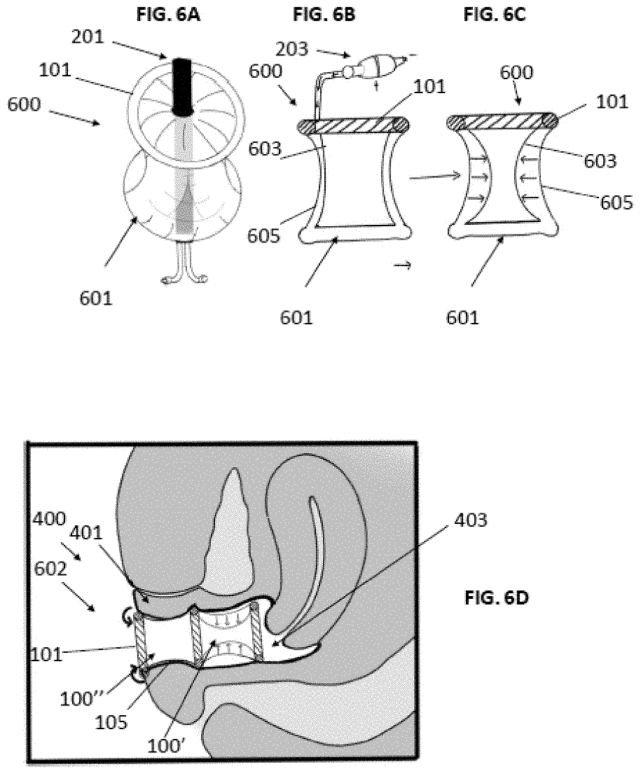

FIGS. 6A-6C show a medical device 600 that may be similarly configured as access device 100, but may further include an inflatable distal end 601. The proximal ring 101 may be a rigid, semi-rigid, or flexible ring. The inflatable distal end 601 may be a part of the sleeve 105 and located at a distal end of sleeve 105. For example, the inflatable distal end 601 may be fluidly connected to the sleeve 105. The inflatable distal end 601 may have an inner wall 603 and an outer wall 605. Similar to medical device 100, when inflated by an inflation device 203 the inner wall 603 may form an airtight seal. Further, once inflated the outer wall 605 may provide a radial force to hold open vaginal walls 401 of an opening in a body. The inflatable distal end 601 may be inflated along with sleeve 105 using the inflation device 203, as described above.

FIG. 6D shows a medical device 602. Medical device 500 may be inverted to form medical device 602. In medical device 602 the inflatable access device 100′ may be a distal device forming the seal and may be positioned entirely in vaginal 400, and thus located distal of the retracing access device 100″. Inflatable access device 100′ may be partially inflatable, such as device medical 600, or completely inflatable. A proximal ring 101 of the retracting access device 100″ may be positioned on an exterior of a body opening (e.g. vagina 400). Inflatable access device 100′ may be connected to the exterior rigid ring 101 via a flexible sleeve 105. Sleeve 105 may be rolled at the proximal ring 101 to reduce any slack in the flexible sleeve 105. The rolling of proximal ring 101 may cause a force that causes the vaginal walls 401 to retract. Inflatable access device 100′ may be inflated either while anchored in the cavity 403 or prior to insertion of medical device 602.

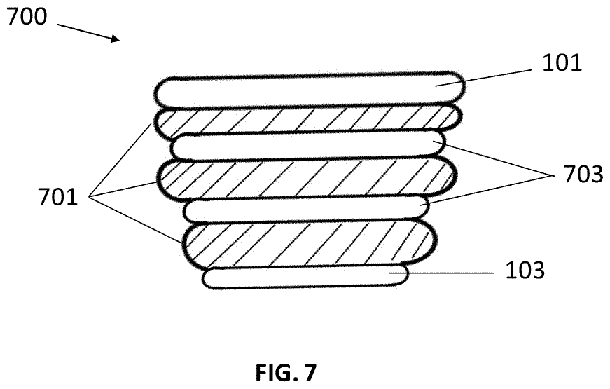

FIG. 7 shows a medical device 700 that includes staggered ribs 701 and middle rings 703. Medical device 700 may further include a proximal ring 101 located at a proximal end of medical device 700, a distal ring 103 located at a distal and of medical device 700. In between proximal rings 101 a distal ring 103, there may be ribs 701 and middle rings 703 that are staggered. In one embodiment, there may be two middle rings 703 and three ribs 701 total. The ribs 701 and middle ring 703 may be inflatable. The ribs 701 may be made of rigid, semi-rigid, or flexible material. Ribs 701 may each be a different type or material. For example, some ribs 701 may be inflatable, while others may be rigid. One or more middle rings 703 may be positioned between the proximal ring 101 and distal ring 103. Each middle ring 703 may have an adjacent proximal rib 701 and an adjacent distal rib 701. Middle rings 703 may be rigid, semi-rigid, or flexible. Each middle ring 703 may be a different type or material.

The rings 101, 103, 703 and ribs 701 may each have varying diameters and thicknesses. This may allow for different rings to be used based on the differing anatomies of different patients. One or more of the rings 101, 103, 703 and ribs 701 may be doubled, meaning of each there may be two each type of ring stacked on itself within the medical devices in order to provide extra stability and potentially double a sealing surface. In another embodiment, three or more rings may be stacked on itself to further increase the height. One or more of the rings 101, 103, 703 and ribs 701 may be inflated or expandable. If expandable, the rings may be inserted into an individual while compressed and able to expand in situ in a patient. Adjacent rings 101, 103, 703 and ribs 701 may be different materials, varying thicknesses, and/or have different rigidity. The proximal ring 101 may have a larger radius than the distal ring 103. Ribs 701 may have a larger radius than adjacent middle rings 703. The Ribs 701 nearest the proximal ring 101 may have the largest diameter of the ribs 701, and the radius of each subsequent rib 701 approaching the distal ring 103 may be smaller than the preceding rib 701. Similarly, the radius of each middle ring 703 may decrease as the middle rings approach the distal ring 103. The ribs 701 and middle rings 703 may provide additional friction to anchor medical device 700 in a body cavity and/or may assist in sealing the medical device 700 in a body cavity. In another embodiment, the proximal ring 101 may have the smallest diameter, the distal ring 103 may have the largest diameter, and the middle rings 703 may increase in diameter from the proximal ring 101 to the distal ring 103.

The rings 101, 103, 703 and ribs 701 may each have varying cross sections. In one example, the rings may have ovular cross sections. This may allow for better stability and an elongated sealing surface. In another example, the rings may have a C-shaped cross section. This may improve the grip or stability of the rings. In another example, the rings may have an inflatable portion circumferential to the primary ring. This may provide additional sealing and support for rings in situ in an opening. In another example, the rings with C-shaped cross sections may include inflatable portions circumferential to the primary ring. This may including filing in the c contour of the rings.

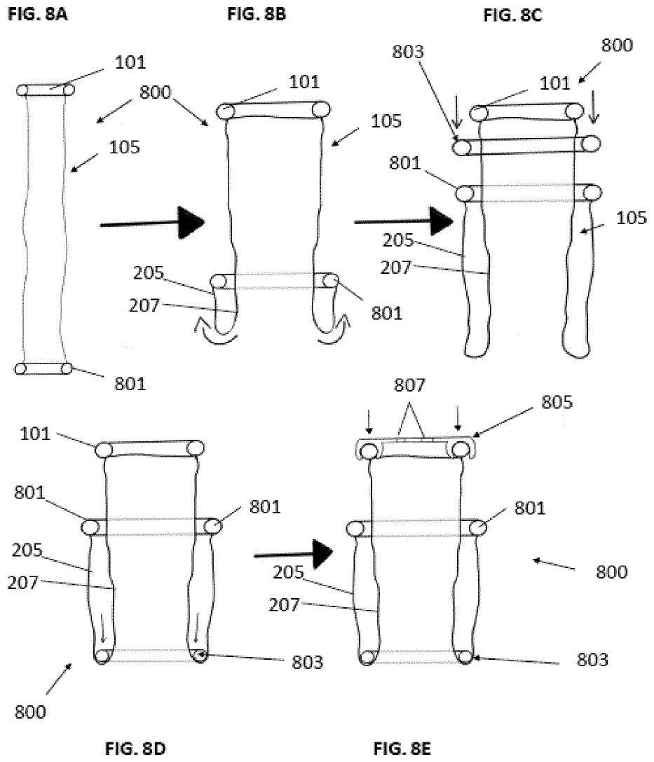

FIGS. 8A-8E show a single sleeve 105 with a ring on each end that may be inverted within itself to create a double layer arrangement medical device 800. This may allow for the distal ring 103 to be a separate component and may allow for use of various ring sizes and shapes.

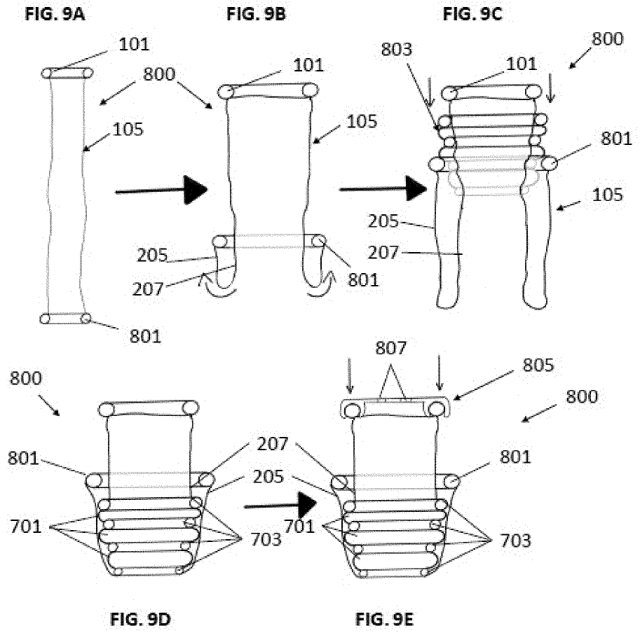

FIG. 8A shows a medical device 800 with a proximal ring 101, a variable internal ring 801, and a sleeve 105. FIG. 8B shows the process of inverting the variable internal ring 801 of medical device 800 around the outer diameter of sleeve 105 to create the double layer sleeve 105 that may include an inner wall 207 and an outer wall 205. FIG. 8C shows a third ring 803 (e.g., a variable internal ring) that may be inserted into the gap between the inner wall 207 and the outer wall 205 of the inverted sleeve 105. The gap may be referred to as the sleeve cavity. Third variable internal ring 801 may be various sizes and types of rings. For example, third ring 803 may be inflatable, rigid, semi-rigid, or flexible. In another embodiment, medical device 800 may include a plurality of third rings 803 that may be inserted into the gap between the outer wall 205 and the inner wall 207 (depicted in FIG. 9A-9E). FIGS. 8D and 8E illustrate the medical device 800 once third ring 803 has been inserted into sleeve cavity and moved to a distal end of the sleeve cavity between the inner wall 207 and the outer wall 205 of the inverted sleeve 105.

FIG. 8E further shows medical device 800 with a cap 805 applied to the proximal ring 101 to facilitate sealing with surgical tools 201. The cap 805 may friction fit around the proximal ring 101 and may have a multitude of access ports 807 for surgical tools 201. Cap 805 may also be snap fit, adhered to the proximal ring 101, or may be attached in any other known manner to the proximal ring. The cap 805 may be rigid, semi-rigid, flexible, or a combination. For example, the proximal surface of the cap may be a flexible film, while the rest of the cap is a rigid plastic. The cap 805 may seal around the proximal ring 101. The access ports 807 may be opened or closed as needed to allow surgical tools 201 access to the body cavity. The access ports 807 may seal around any surgical tool 201 inserted.

FIGS. 9A-9E shows medical device 800 of FIGS. 8A-8E with a plurality of third rings 803. The inverted sleeve 105 may be compatible with a variety of ring designs and multiple third rings 803 between the inner wall 207 and outer wall 205 of the inverted sleeve 105. For example, FIGS. 9C-9E show that the third rings 803 may be substituted for the staggered rib 701 and middle ring 703 of medical device 700 of FIG. 7 wherein ribs 701 may be inflatable. The staggered rib 701 and middle ring 703 of medical device 700 may similarly be inserted in a gap between outer wall 205 and inner wall 207 as described in FIG. 8A-8E. FIG. 9E shows medical device 800 with a cap 805 with access ports 807, similar to FIG. 8E.

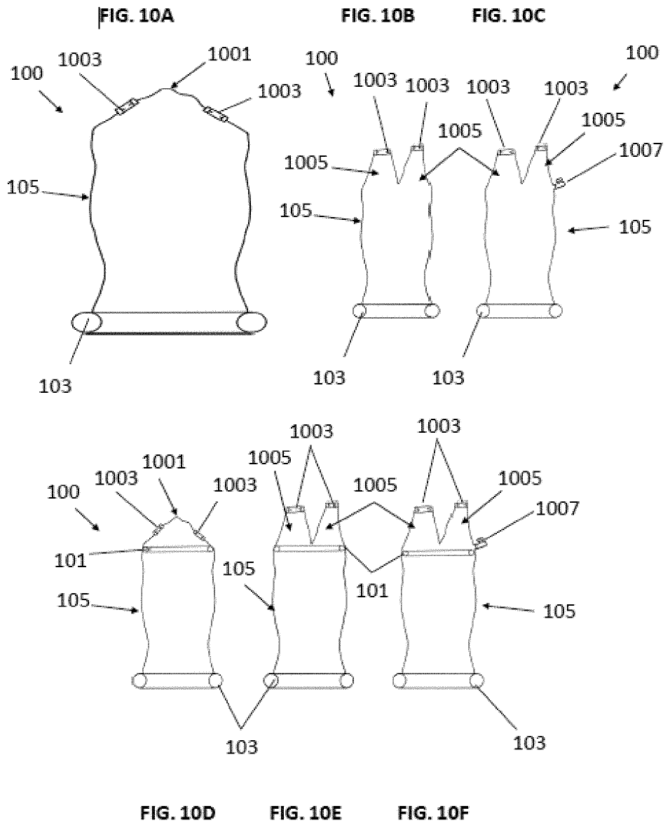

FIG. 10A shows another embodiment of the medical device 100 that includes a single layer sleeve 105, a fixed distal ring 103, and a proximal enclosed sleeve end 1001. The enclosed sleeve end 1001 may incorporate multiple valves 1003 capable of receiving surgical instruments 201. The valves 1003 may be in an open state capable of accepting surgical instruments 201 or in a closed state, sealing the valve without a tool 201. While in an open state, the valves 1003 may seal around surgical instruments 201 inserted through the valve 1003.

FIGS. 10B shows medical device 100 with individual chutes 1005 in the sleeve 105 for each valve 1003. While FIGS. 10B-10C and FIGS. 10E-10F show two chutes 1005, any number of chutes 1005 may be at the proximal end of the device. For example, the medical device 100 may include a chute 1005 for every valve 1003 included in sleeve 105. The chutes 1005 may be portions of the same sleeve 105 that narrow to valves 1003 at the proximal end of the medical device 100. Each chute 1005 within sleeve 105 may be the same size or different sizes. For example, a first chute 1005 may be sized to fit a first surgical tool 201, while a second chute 1005 may be sized to fit a second surgical tool 201. Similarly, the valves 1003 may be the same size or different sizes. For example, each of the valves 1003 may be sized to fit a specific surgical tool 201. The openings of valves 1003 may be adjustable to fit different surgical tools 201 or to assist in sealing.

FIG. 10C shows medical device 100, as described in FIG. 10A-10B, further including an insufflation port 1007. The insufflation port 1007 may be used with inflation device 203 to insufflate a body cavity, adjust pressure within the cavity, inflate the sleeve 105, deflate the sleeve 105, or otherwise adjust fluid within the medical device 100 or body cavity. Any embodiment of the medical device 100 may include an insufflation port 1007.

FIGS. 10D-10E show a medical device 100, as described in FIGS. 10A-10C that further includes a proximal ring 101 inside the sleeve 105 located closer to the proximal enclosed sleeve end 1001. The proximal ring 101 may provide structure and support for the sleeve 105. Similar to FIGS. 10A-10C, the device 100 of FIGS. 10D-10F may include distal ring 103, sleeve 105, valves 1003, chutes 1005, and insufflation port 1007. The sleeve 105 may be a single sleeve or a double sleeve with an outer wall 205 and an inner wall 207. If device 100 is in a double sleeve 105 configuration, the double sleeve 105 may be inflated for additional rigidity and/or support. Further, the insufflation port 1007 may be used to inflate and/or insufflate the body cavity.

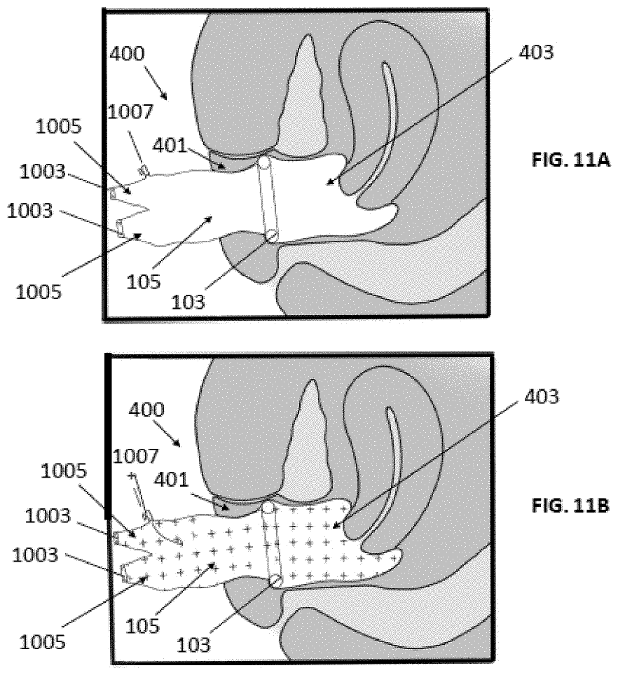

FIGS. 11A and 11B show medical device 100 from FIG. 10C while in use. The medical device 100 may have distal ring 103, sleeve 105, enclosed proximal end 1001, valves 1003, chutes 1005, and insufflation port 1007. The distal ring 103 may be anchored in the vaginal wall 401 to hold the device 100 in place within the vagina 400. FIG. 11B shows that the medical device 100 and cavity 403 may be insufflated through the insufflation port 1007 in order to maintain pneumoperitoneum pressure within the vaginal cavity 403 while in use.

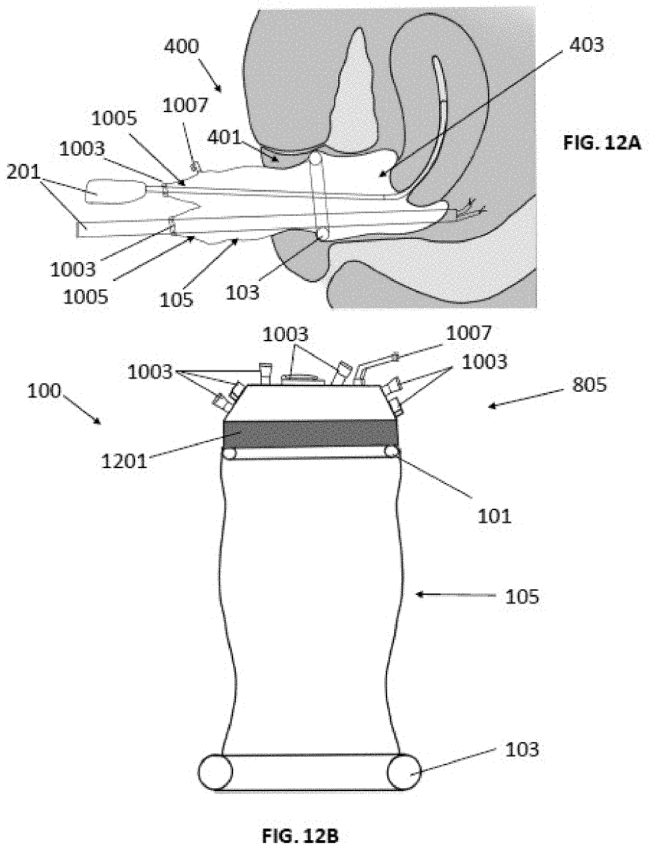

FIG. 12A shows the medical device 100 as while in use. The medical device 100 may have a distal ring 103, the enclosed proximal end 1001, valves 1003, and chutes 1005. Surgical tools 201 may be inserted through valves 1003 to access the vaginal cavity 403. Each surgical tool 201 may be a different tool 201 for a surgical procedure. Valves 1003 may be sized differently to accommodate different tools 201. Valves 1003 may seal around each tool 201.

FIG. 12B shows the medical device 100 with a cap 805. The cap 805 may have a plurality of valves 1003. In one embodiment, the cap may have a wider base as a distal end and the cap may narrow in diameter at a most distal end of the cap. Valves 1003 may protrude perpendicular or at an angle from the proximal ring 101. Further, valves 1003 may be located at a proximal end of cap 805 or along the sides of cap 805. Valves 1003 may be rigid, semi-rigid, or flexible. The cap 805 may include a base 1201 that is capable of coupling to the proximal ring 101. The base 1201 may create an airtight seal around the proximal ring 101. The cap 805 and/or the base 1201 may be rigid, semi-rigid, or entirely flexible.

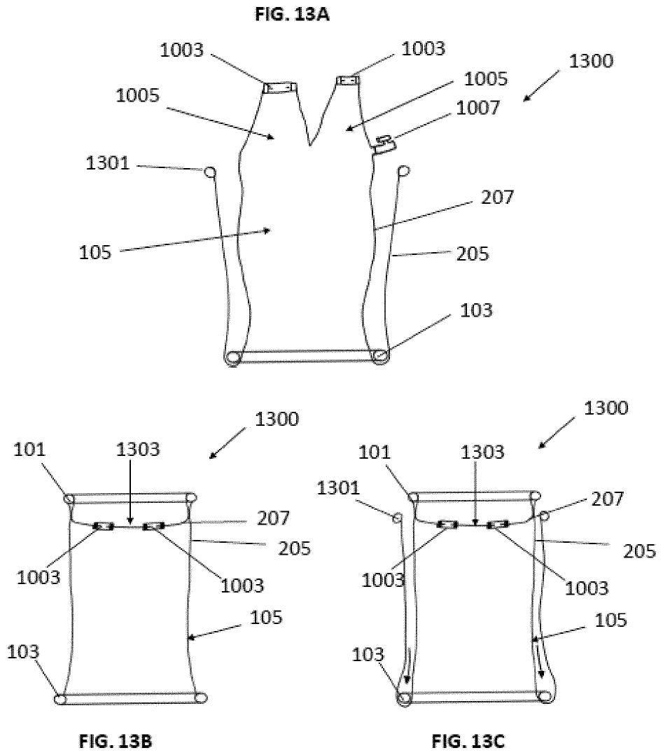

FIG. 13A shows an embodiment of a medical device 1300 that may include the chutes 1005 for each instrument valve 1003 at a proximal end of medical device 1300. Medical device 1300 may be an alternative embodiment of medical device 100. Medical device 1300 may include an inverted sleeve 105 and distal ring 103 described in FIGS. 8A-8E and the chutes 1005 and instruments valve 1003 described in FIGS. 10A-10F. The medical device 1300 may thus include the variable internal ring design with an enclosed sleeve valving system. Distal ring 103 may be capable of being positioned in a body cavity and may anchor medical device 1300 in a body opening. For example, distal ring 103 may contact tissue defining the body cavity and may anchor medical device 1300 within a body opening. The inverted sleeve 105 may be used with the distal ring 103. The distal ring 103 may be located between the inner wall 207 and outer wall 205 of the inverted sleeve 105. Distal ring 103 may be any of the discussed ring designs, such as a single ring or a staggered ring design with inflatable ribs 701. In one example, an inverted sleeve end 1301 may refer to an end of sleeve 105 located at an opposite end of the sleeve from chutes 1005. The inverted sleeve end 1301 may be wrapped around the distal ring 103 and pulled from outside a body cavity 403 to tighten the sleeve 105. The inverted sleeve end 1301 may be a ring. Alternatively, the inverted sleeve end may be structures not connected to each other. The sleeve 105 may be a single wall that tightens when inverted sleeve end 1301 is pulled in a proximal direction, which may retract a cavity wall, such as vaginal wall 401. The sleeve may also be a double sleeve wall that may be inflated to retract a vaginal wall 401.

FIGS. 13B and 13C show an alternative design medical device 1300 in which the sleeve 105 extends from the distal ring 103 to wrap around the outer radius of the proximal ring 101 into the inside of the sleeve 105. The inner wall 207 of the sleeve 105 may be coupled, such as by welding or by an adhesive, to the outer wall 205 to create a sleeve area 1303 for one or more valves 1003. The sleeve area 1303 may provide additional structure and support that is internal to the sleeve 105 and offset from the proximal ring 101. FIG. 13C shows an embodiment with the inverted sleeve from FIG. 13A extending around the distal ring 103.

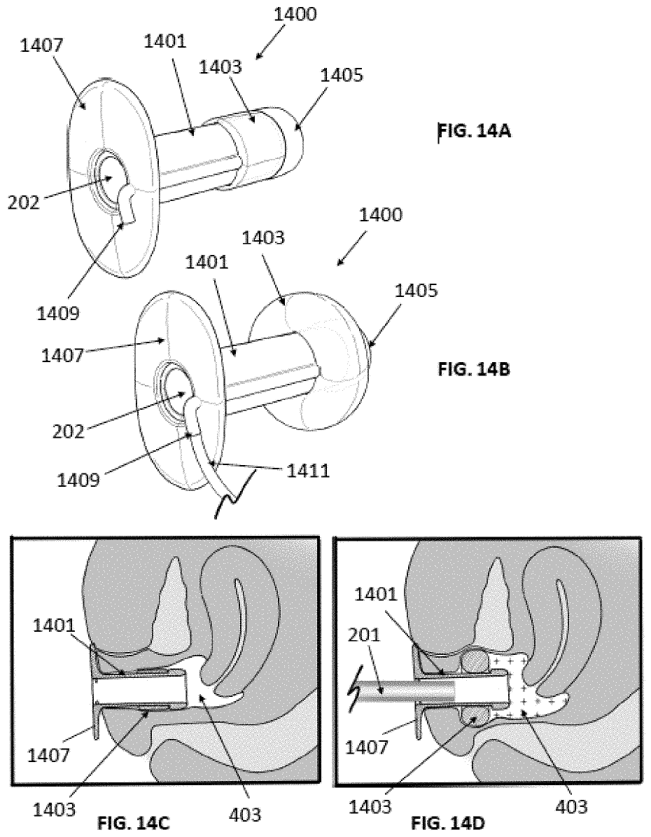

FIGS. 14A-D show a medical device 1400. FIG. Medical device 1400 may include a rigid or semi-rigid base element 1401 with a proximal end 1407 and a distal end 1405. The base element 1401 may be cylindrical in shape. A lumen 202 may extend from proximal end 1407 of a rigid or semi-rigid base element 1401 through to a distal end 1405. The proximal end 1407 may be flared, tapered, or otherwise structured as a ring. The distal end 1405 of the medical device 1400 may include an inflatable element 1403. Inflatable element 1403 may wrap around a section of the distal end 1405. Inflatable element 1403 may be capable of receiving a gas through one or more openings to expand and protrude radially from base element 1401. The inflatable element 1403 be capable of being insufflated. The medical device 1400 may include a port 1409 on the proximal end 1407 used to inflate or deflate the inflatable element1403 of the medical device 1400. The port 1409 opening may be located at the proximal end 1407 and extend along base element 1401 until the port 1409 contacts the inflatable element 1403. Port 1409 may be capable of being fluidly connected to a device 1411 capable of inflating the inflatable element 1403.

FIG. 14A shows the inflatable element 1403 when not inflated and FIG. 14B shows the inflatable element 1403 when inflated. The inflatable element 1403 may be inflated by an inflation device 203. Inflating the inflatable element 1403 while in cavity 403 may secure the device within the vagina 400 and/or create a seal within the vagina 400. The size of the medical device 1400 and inflatable element 1403 may be changed based on the anatomy of the patient. The rigid or semi-rigid element 1401 may be completely disposed within an opening of the patient (e.g., vagina 400). The inflatable element 1403 may be made of many smaller inflatable portions or one large inflatable portion. When inflated, inflatable element 1403 may form the shape of a torus.

FIGS. 14C and 14D show the medical device 1400 with inflatable element 1403 in use when not inflated (FIG. 14C) and when inflated (FIG. 14D), respectively. FIG. 14D shows an inflated inflatable element 1403 sealing cavity 403 to maintain pressure within the cavity. One or more instruments 201 or other shafts may be inserted through the lumen 202 of the device 1400 to access the cavity 403.

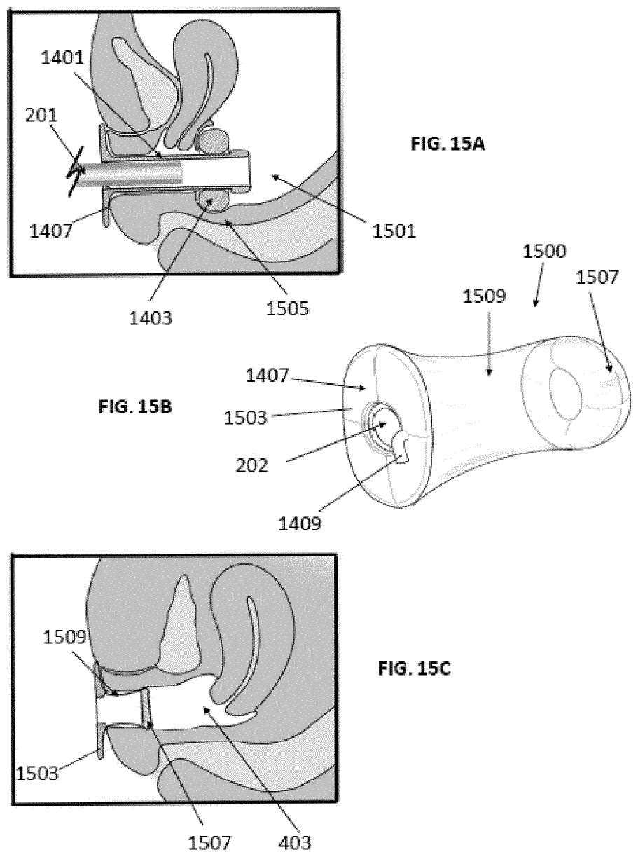

FIG. 15A shows the medical device 1400 of FIGS. 14A-14D inserted through a colpotomy 1501. Colpotomy 1501 may be an incision or opening within the vagina 400. Inflatable element 1403 may be capable of holding open the colpotomy wall 1505 when insufflated. This may allow tool 201 to access the colpotomy for a surgical procedure.

FIG. 15B shows medical device 1500. Medical device 1500 may include a rigid or semi-rigid cap 1503 at the proximal end 1407 that is connected to an inflatable distal ring 1507 via a flexible sleeve 1509. The inflatable distal ring 1507 may be equivalent to inflatable element 1403. Flexible sleeve 1509 may be an inflatable flexible sleeve or a non-inflatable flexible material. A lumen 202 may extend from the rigid or semi-rigid cap 1503 through the flexible sleeve 1509 through the inflatable distal ring 1507. Port 1409 may be used to inflate inflatable distal ring 1507 and/or flexible sleeve 1509. FIG. 15C shows the medical device 1500 seated in a patient's anatomy in an inflated state. The inflatable distal ring 1507 may hold the medical device 1500 in place

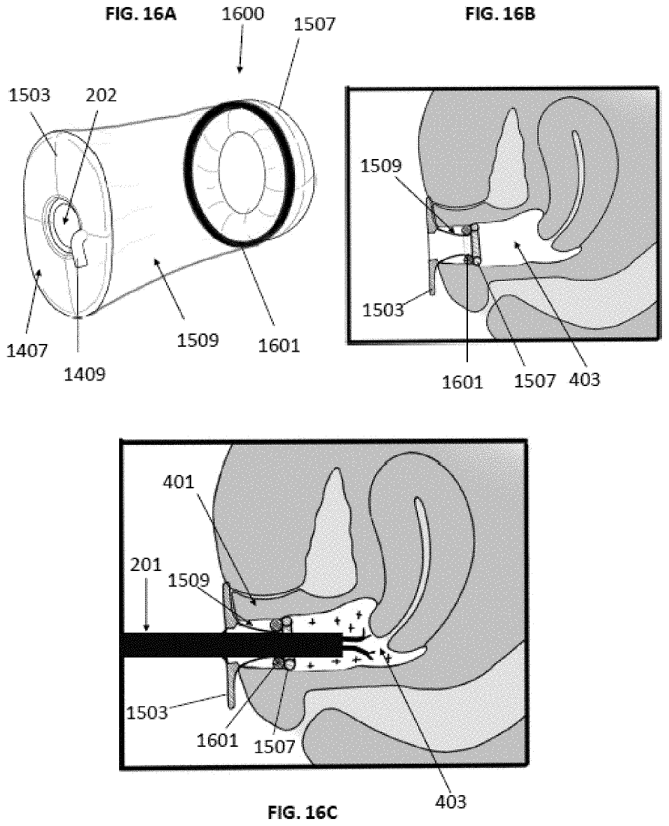

FIG. 16A shows medical device 1600 that includes a rigid or semi-rigid cap 1503 at the proximal end 1407 that is connected to an inflatable distal ring 1507 at the distal end via a flexible sleeve 1509, similar to medical device 1500 described in FIGS. 15B-15C. Unlike medical device 1500, medical device 1600 may include a rigid ring 1601. The rigid ring 1601 be in the shape of a torus and located on the exterior of flexible sleeve 1509 immediately adjacent to the inflatable ring 1507 at the distal end. The presence of the rigid ring 1601 may help to structurally support the inflatable ring 1507. Alternatively, rigid ring 1601 may be semi-rigid. FIG. 16B shows medical device 1600 seated in a patient's anatomy. The inflatable distal ring 1507 may anchor and/or seal the medical device 1600 within the cavity 403, while ring 1601 provides support for sleeve 1509. FIG.16C shows the medical device 1600 seated in a patient's anatomy while in use with a tool 201. Tool 201 may access cavity 403 through lumen 202. Rigid ring 1601 and inflatable distal ring 1507 may seal lumen 202 around tool 201 and vaginal wall 401 around sleeve 1509 to maintain pressure within a patient's cavity 403.

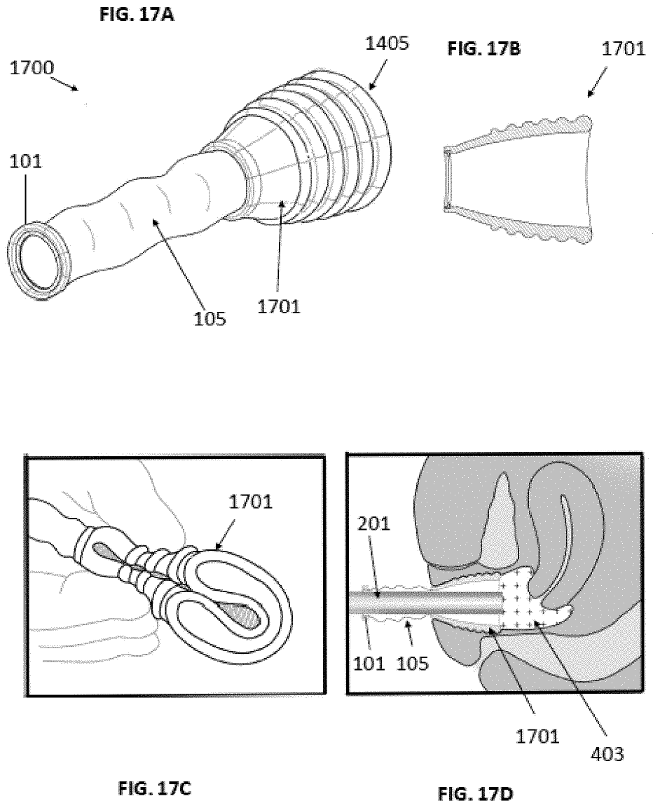

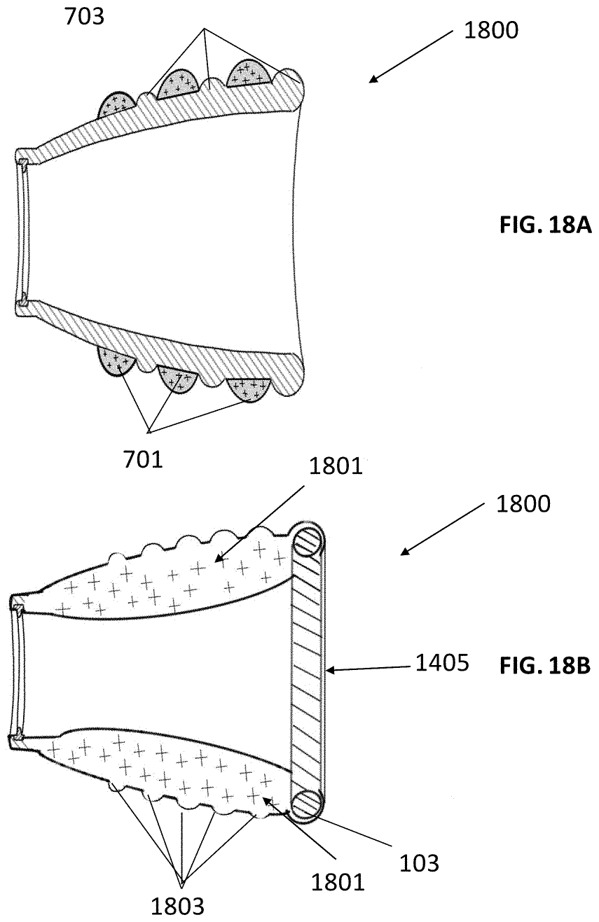

FIGS. 17A-17D show medical device 1700 that may include a proximal ring 101, sleeve 105, and distal ring section 1701. The distal ring section 1701 may be a separate piece (e.g., attachable or non-attachable) or part of the same piece as the rest of the medical device 1700. The distal ring section 1701 may consist of multiple semi-rigid rings connected to each other. These rings may have increasing radii as the rings approach the distal end 1405. As shown in in FIG. 17C, the medical device 1700 may be inserted into the vagina 400 by folding the distal ring section 1701 outside the patient, inserting the folded distal ring section into the patient through a body cavity 403, and expanding the distal ring section 1701 when medical device 1700 is positioned correctly. Expansion of distal ring section 1701 may occur automatically to pressure no longer being applied to the exterior of distal ring section 1701. The series of rings and/or ridges may help secure the device in place and/or seal the cavity 403. The distal ring section 1701 may be cut or trimmed to reduce the length and number of rings as needed to match a patient's anatomy. The proximal ring 101 may be able to receive one or more valves 1003 to allow instruments 201 to pass through the medical device 1700 while maintaining a seal. In another embodiment, medical device 1700′s distal ring section 1701 may be may ring section device 1800 (shown in FIG. 18A-18B). Ring section device 1800 is discussed in further detail below.

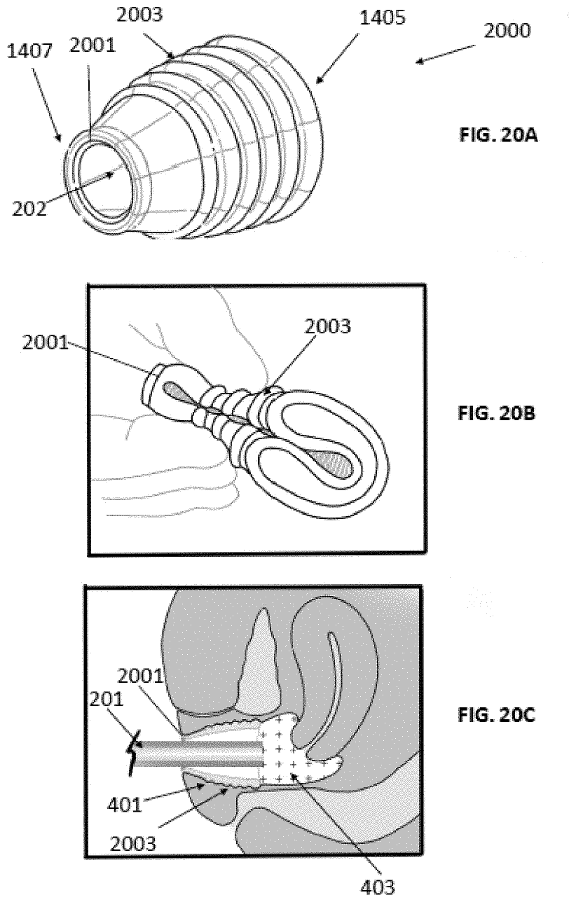

FIG. 18A shows a ring section device 1800 similar to distal ring section 1701 of FIGS. 17A-17D. Ring section device 1800 includes a plurality of inflatable ribs 701 between a plurality of rings 703. This configuration of ring section device 1800 may aid in sealing and anchoring the device by providing less trauma to the patient while ring section device 1800 is inserted/maintained in cavity 403. The rings 703 may be rigid, semi-rigid, or flexible. Ring section device 1800 may be attached to a sleeve 105 with a proximal ring 101, as distal ring section 1701 is in FIGS. 17A-17D. Alternatively, ring section device 1800 may be inserted into a patient's anatomy as its own access device (see FIGS. 20A-20C).

FIG. 18B shows a ring section device 1800. In this embodiment, the ring section device 1800 may be inflatable and include an inflatable distal ring structure 1801. When inflated, the inflatable distal ring structure 1801 may expand outward. The inflatable rings 1803 may expand when inflated. Inflatable rings 1803 may be rings encircling inflatable distal ring structure 1801 or ribs partially encircling inflatable distal ring structure 1801. Inflatable rings 1803 may be portions of the distal ring structure 1801 or separate rings. The inflatable nature of ring section device 1800 may aid in insertion, sealing, and/or anchoring the ring section device 1800 in the patient. The ring section device 1800 may include a rigid or semi-rigid distal ring 103 at the distal end 1405 to provide structure for the inflatable ring section device 1800.

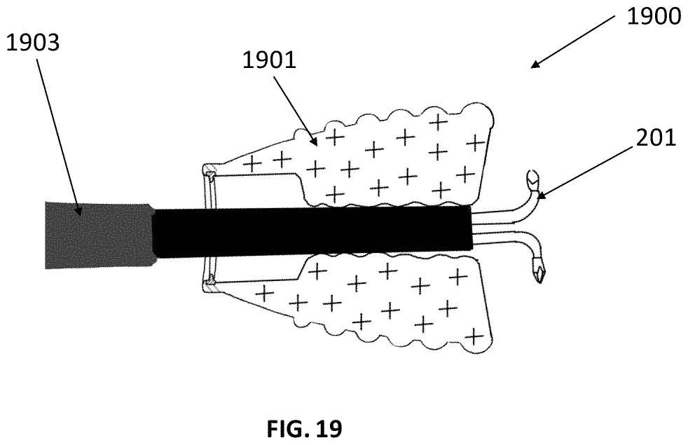

FIG. 19 shows an inflatable ribbed device 1900. Inflatable ribbed device 1900 may include inflatable section 1901 that may expand both outward and inwards. Expanding outwards may aid in insertion, sealing and/or anchoring the inflatable ribbed device 1900 in the patient. Inflatable ribbed device 1900 may be capable of receiving surgical tools through a lumen. When inflatable section 1901 expands inward, this may aid in sealing around a surgical tool 201. Inflatable section 1901 may be inflated after the inflatable ribbed device 1900 is positioned within an opening (e.g., vagina 400). Inflatable section 1901 may be kept at a constant pressure or pressure may be adjusted based on the anatomy of the patient and one or more surgical tools 201 inserted. Adjusting the pressure of inflatable section 1901 may help with sealing the device along with any inserted instruments 201.

A surgical tube 1903 may pass through inflatable ribbed device 1900 or other medical devices 100 described herein. The surgical tube 1903 may be a hollow tube that surgical tools 201 pass through to access a body cavity. The surgical tube 1903 may seal around the surgical tool at the proximal end, the distal end, or in between. Surgical tube 1903 may be rigid, semi-rigid, or flexible. Inserting surgical tools 201 through a surgical tube 1903 may be preferable to using surgical tools 201 alone. For example, different surgical tools 201 may be differently sized or may move while in use, which may create difficulties in sealing around the surgical tools 201. Surgical tubes 1903 of a known diameter may be used in conjunction with medical devices 100 and inflatable ribbed device 1900. Medical device 100 and inflatable ribbed device 1900 may be designed to create an airtight seal around surgical tubes 1903. Surgical tubes 1903 may allow surgeons to manipulate surgical tools 201 freely without creating breaches of the airtight seal. Surgical tubes 1903 may be specialized tubes designed to be used with access devices, such as medical devices 100 and inflatable ribbed device 1900. A surgical tube 1903 may also be a surgical tool 201, such as an endoscope, that is able to hold other surgical tools 201, allow other surgical tools 201 to pass through, or otherwise allow other surgical tools 201 access to body cavity. For example, an endoscope may act as a surgical tube 1903 while a suction or cutting tool 201 may pass through the endoscope body to be used within the body cavity the endoscope is viewing.

FIGS. 20A-20C show a medical device 2000 similar to medical device 1700 of FIGS. 17A-17D but without a sleeve 105. FIG. 20A shows a medical device 2000 with a lip seal 2001 and a distal ring section 2003. The distal ring section 2003 may consist of multiple rings connected to each other. The rings of distal ring section 2003 may be semi-rigid, flexible, or a combination. In some examples, distal ring section 2003 may be inflatable and expand outward and/or inward. The rings on distal ring section 2003 may have increasing radii as the rings approach the distal end 1405. Instead of sleeve 105, a lip seal 2001 may be formed at the proximal end 1407 of the medical device 2000. This lip seal 2001 may seal the device against a single tool 201. Alternatively, there may be multiple lip seals 2001 for multiple instruments 201. Instruments may pass through lumen 202 to access a body cavity 403. FIG. 20B shows medical device 2000 with a semi-rigid distal ring section 2003. The semi-rigid distal ring section 2003 may be folded or adjusted so that medical device 2000 may be inserted into a patient. After insertion, the medical device 2000 may return to a neutral non-folded state. FIG. 20C shows medical device 2000 inserted within vagina 400 after medical device 2000 has returned to the neutral non-folded state. The rigidity of the distal ring section 2003 may retract the vaginal walls 401.

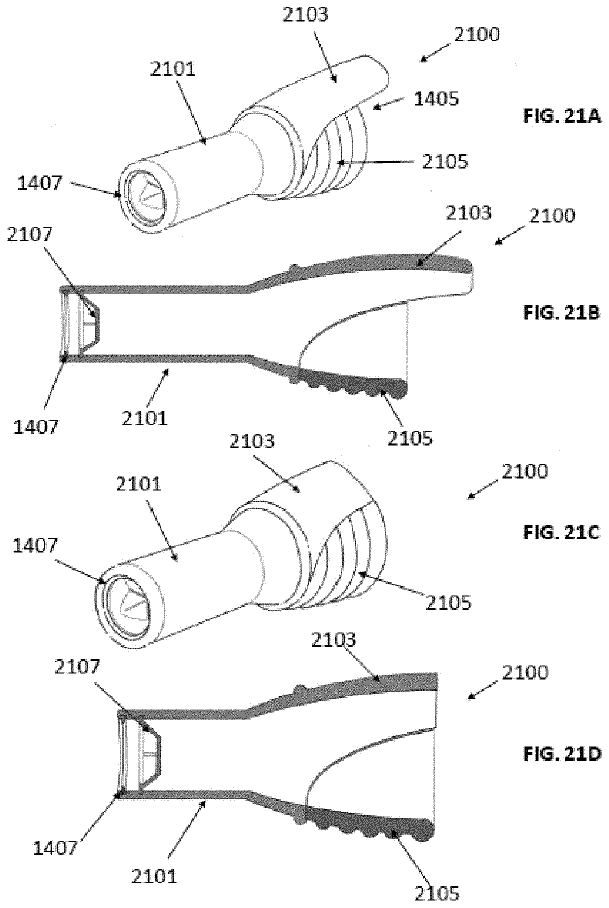

FIGS. 21A and 21B show a medical device 2100 with a rigid proximal end 1407 and a sleeve portion 2101. The sleeve portion 2101 may include a hood 2103 on at least a portion of the distal end 1405. Hood 2103 may extend from sleeve portion 2101 at the upper side of the distal end 1405 of the sleeve portion 2101. Hood 2101 may have a single rib that encircles the sleeve portion and is located adjacent to sleeve portion 2101 Hood 2103 may be rigid. Hood 2103 may extend further distally than lower side 2105. Lower side 2105 may be semi-rigid. Lower side 2105 may me made of multiple rings or ribs connected to each other. Lower side 2105 may be inflatable. The rigid hood 2103 may extend further distally than lower side 2105 in order to assist in accessing one side of the vagina during a colpotomy as this may minimize obstruction. The rigid proximal end 1407 may include an internal rigid cannula 2107 for tool 201 insertion. FIGS. 21C-21D show an embodiment of medical device 2100 where hood 2103 does not extend further than lower side 2105.

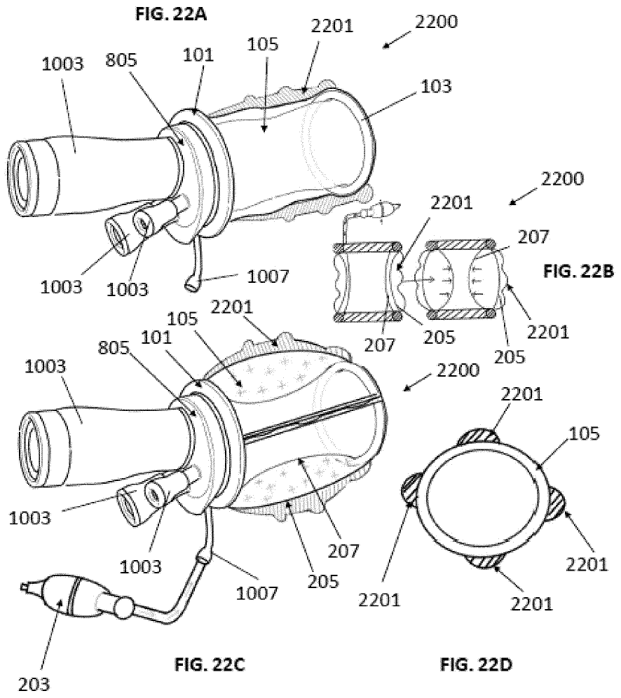

FIGS. 22A-D show a medical device 2200 with a cap 805 and an inflatable sleeve 105. FIG. 22A shows medical device 2200 while deflated. Inflatable sleeve 105 may be a flexible inflatable sleeve 105 with four semi-rigid, ribbed strips 2201 spaced at 90 degree intervals around the circumference of sleeve 105. The ribbed strips 2201 aid anchoring of the medical device 2200 within the vagina 400. The ribbed strips 2201 may extend from the proximal ring 101 along the sleeve 105 to the distal ring 103. The cap 805 may include one or more valves 1003 of various sizes. Further, the medical device 2200 may include an insufflation port 1007 that is capable of insufflating sleeve 105. Insufflation port 1007 may be located at a proximal end of medical device 2200.

FIG. 22B shows the medical device 2200 being inflated. The outer wall 205 may expand radially outward and the inner wall 207 may expand radially inward when sleeve 105 is inflated.

FIG. 22C shows medical device 2200 while inflated. The sleeve 105 may be inflated by an inflation device 203 at insufflation port 1007. The inflatable sleeve 105 may have an inner wall 207 and an outer wall 205. When inflated, sleeve 105 may creates a seal that maintains pneumovagina/pneumoperitoneum pressure between the outer wall 205 and the anatomy (e.g., vaginal walls) and as well as the inner wall 207 and any surgical tools 201 which are inserted. FIG. 22D shows a front view of medical device 2200. Four ribbed strips 2201 may be positioned at 90 degree intervals around the circumference of sleeve 105. Each of the ribbed strips 2201 may extend/protrude radially further than the radius of proximal ring 101 and distal ring 103. Alternatively, medical device 2200 may include different amounts of ribbed strips 2201, such as eight ribbed strips 2201 located at 45 degrees.

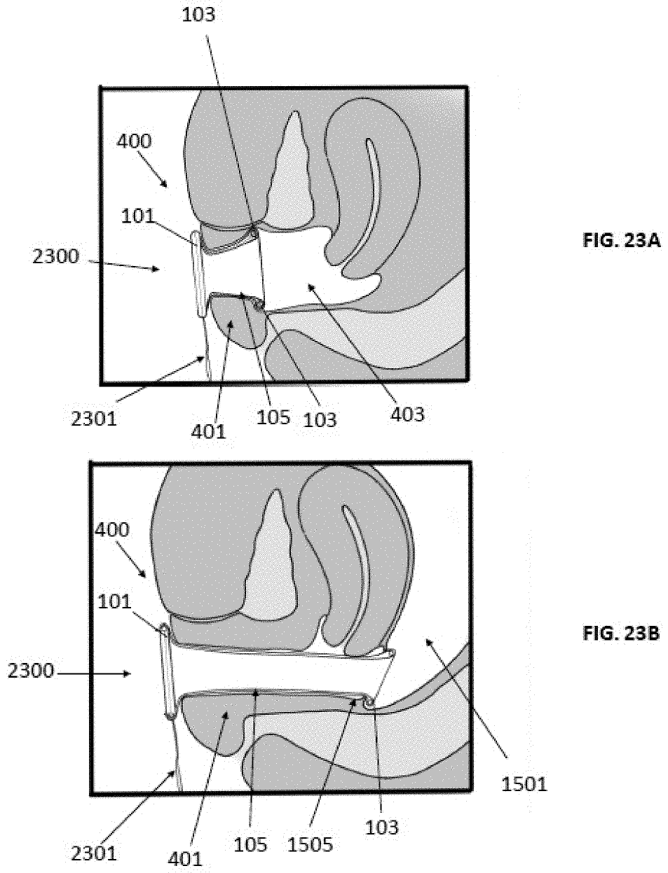

FIG. 23A and FIG. 23B show a medical device 2300 in situ in a vagina 400 or a colpotomy 1501, respectively. Medical device 2300 may have a double sleeve 105, a proximal ring 101, a distal ring 103, and a base retractor 2301. The base retractor 2301 may be a length of material, such as string or a plastic length, that when pulled tightens the flexible sleeve 105. When medical device 2300 is positioned in a patient, tightening the sleeve 105 may cause sleeve 105 to push against and retract either the vaginal wall 401 or the colpotomy wall 1505.

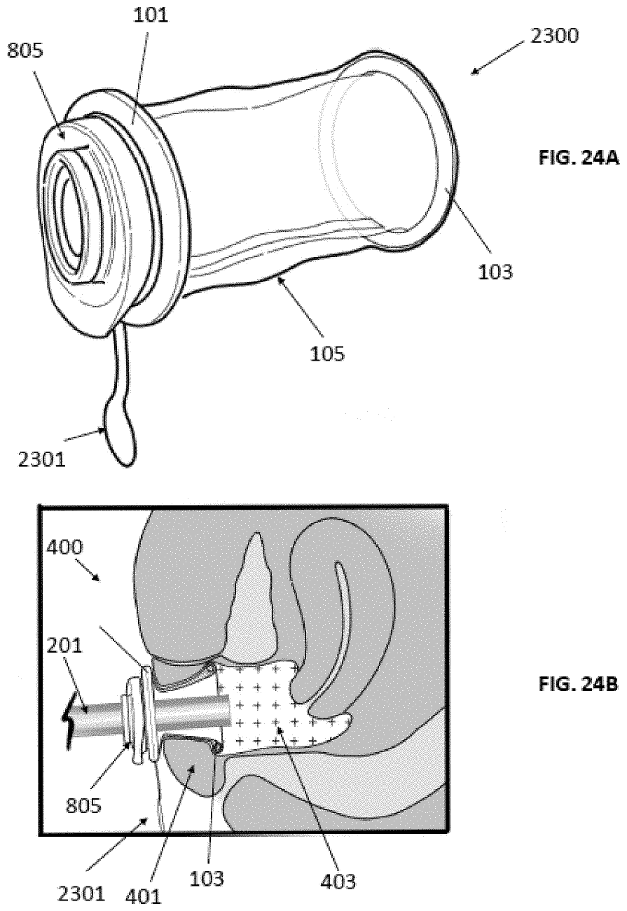

FIG. 24A shows the medical device 2300 with a valve cap 805 attached to the proximal end of the medical device 2300. The medical device 2300 may be in a double sleeve 105 configuration, as described above. The valve cap 805 may seal around one or more surgical tools 201. Alternatively, the valve cap 805 may have a single opening that is designed to seal around a surgical tube 1903, as described above. FIG. 24B shows the medical device 2300 in use. The medical device 2300 may have a tool 201 or surgical tube 1903 inserted through it into the vagina 400. The valve cap 805 may create a seal with the tool 201 or tube 1903 while the tool 201 is being used. Valve cap 805 may also create a seal with proximal ring 101. This may allow medical device 2300 to maintain pressure within cavity 403.

FIG. 25A shows the medical device 2300 with a valve cap 805 that may include multiple valves 1003. The valves 1003 may be rigid, semi-rigid, or flexible. Each valve 1003 on the valve cap 805 may be different materials and sizes. Each valve 1003 on the valve cap 805 may be opened or closed independent from the other valves 1003. The largest valve 1003 of the valve cap 805 may be a main valve 2501. The main valve 2501 may have a flexible film extension to offset the bulk of the valve housing. FIGS. 25B-25D show the device medical 2300 anchored respectively within a vagina 400 or colpotomy 1501. An tool 201 or tube 1903 may be inserted through the main valve 2501 and main valve 2501 may seal around tool 201 or tube 1903. The tool 201 may create a seal while in situ in the vagina or through a colpotomy 1501 to maintain pressure within the cavity. In FIG. 25D, additional instrument 2503, which may be a uterine manipulator, may be located in a valve 1003 of cap 805. The additional instrument 2503 may be separate of sleeve 105.

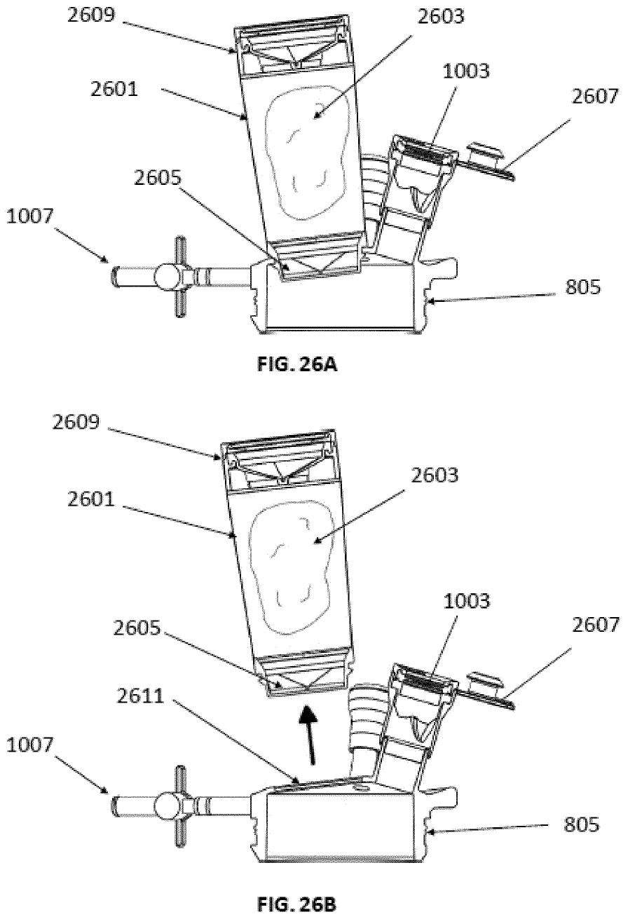

FIGS. 26A and 26B show an embodiment of a cap 805 with a detachable container 2601. Detachable container 2601 may sealably couple to valve cap 805 at an opening in the valve cap 805. The opening that detachable container 2601 attaches to the valve cap 805 may be the largest opening in the valve cap 805. Detachable container 2601 may be a container with a coupling side 2605 and a proximal side 2609. Detachable container 2601 may be a flexible plastic sleeve. Alternatively, detachable container 2601 may be rigid, semi-rigid, flexible, or a combination of materials. For example, coupling side 2605 and proximal side 2609 may be rigid, while the container of detachable container 2601 may be a flexible plastic sleeve.

The coupling side 2605 may create an air-tight seal when coupled to cap 805. Coupling side 2605 may couple to cap 805 by, for example, a friction fit, a snap fit, screw threads, or similar known temporary coupling means. Coupling side may be sealed so that the detachable container 2601 is sealed from the cap 805. In some examples, proximal side 2609 have a vacuum or other source of suction. Alternatively, proximal side 2609 may have an opening where a suction source may be attached to apply suction. Proximal side may have a seal 2607 similar to seals 2607 for valves described below.

FIG. 26A-26B further depicts tissue 2603 located within the detachable container 2601. Many operations require the removal of tissue 2603 within a patient. During an operation, tissue 2603 may be removed from within a body cavity, such as a vaginal cavity 403 or colpotomy 1501. The removal of tissue 2603 may occur from suction. Tissue 2603 may be placed into detachable container 2601 by the suction source in the proximal side 2609. Alternatively, tissue 2603 may be removed from a body cavity by any other traditional methods. FIG. 26B shows a sealed detachable container 2601 with removed tissue 2603 detached from cap 805. Cap 805 may also seal the port 2611 that detachable container 2601 couples to, so that when detachable portion is detached, the cap 805 may maintain the air-tight seal with the access device 100.

A cap 805 may have one or more valves 1003 for providing access to surgical tools 201 or surgical tubes 1903. Valves 1003 on the cap 805 may extend proximally from the cap 805. Valves 1003 may also extend at a non-90 degree angle from the surface of the cap 805. Alternatively, valves 1003 may be sealable gaps in the cap 805 that do not extend from the cap 805. Valves 1003 may be partially sealable, such as around a tool 201. Valves 1003 may also be completely sealed while not in use, for example by a removable valve seal 2607. Valve seal 2607 may be attached to valve 1003 by a hinge. Valve seal 2607 may also be a separate piece only coupled to valve 1003 when the valve 1003 is being completely sealed. Valve seal 2607 may fit into valve 1003 to provide an air-tight seal when the valve 1003 is not in use.

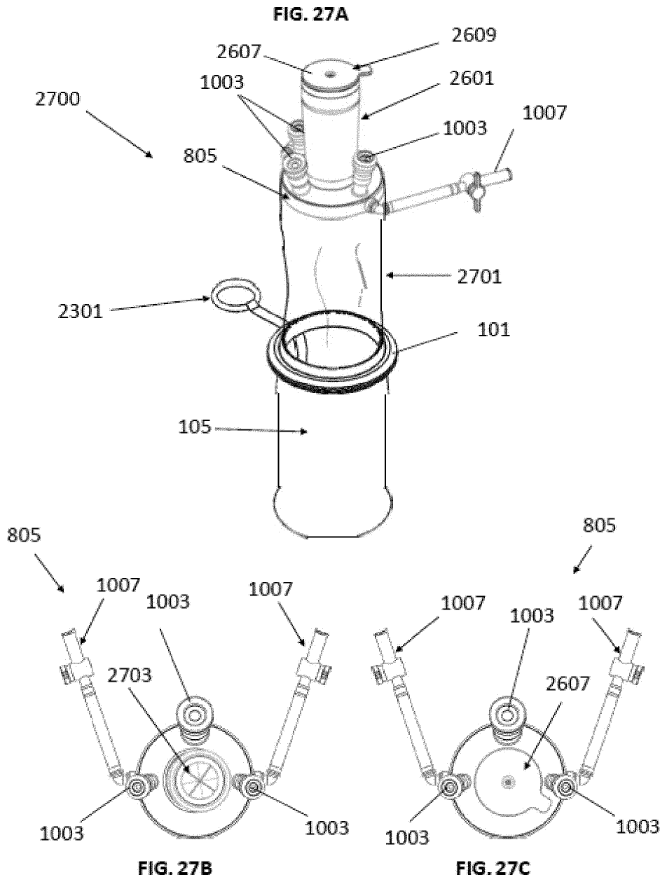

FIGS. 27A-27C show medical device 2700 where the valve cap 805 is attached to a film sleeve 2701 which is attached to a proximal ring 101 of a medical device 100. Film sleeve 2701 may be attachable or detachable from proximal ring 101 and/or retractor 2301. As described above, retractor 2301 may be pulled to tighten, shorten, or otherwise adjust sleeve 105. The film sleeve 2701 may be a flexible plastic. The film sleeve 2701 may be attachable and detachable from the proximal ring 101 and the valve cap 805. The film sleeve 2701 may be used to facilitate sample removal. The valve cap may include multiple valves 1003 for multiple instruments 201 and/or multiple insufflation ports 1007. The valve cap 805 may include a detachable container 2601, as described above.

FIG. 27B shows a top view of the valve cap 805 described in FIG. 27A when detachable container 2601 is detached from valve cap 805. A seal 2703 may be formed when detachable container 2601 is detached so that pressure and fluids may be maintained within the medical device 2700. FIG. 27C shows a top view of the valve cap 805 when detachable container 2601 is attached. Proximal side 2609 of the detachable container 2601 may have a seal 2607 to seal the proximal side of the detachable container 2601. This seal 2607 may be attached to the detachable container 2601 on a hinge or similar connection, as described above.

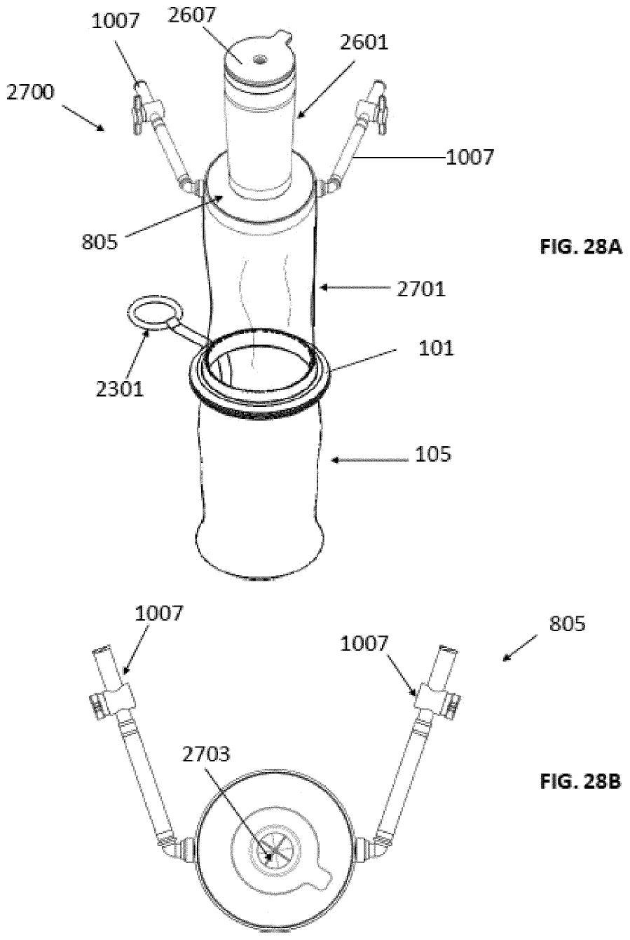

FIG. 28A-28B shows medical device 2700 where the valve cap 805 may include a single detachable container 2601 and two insufflation ports 1007. In one embodiment, the sleeve container may protrude proximally from the cap 805. One or more insufflation ports 1007 may be located on side of cap 805 or film sleeve 2701 and protrude outward. Insufflation ports 1007 may be located 180 degrees apart from each other along the circumference of cap 805 or film sleeve 2701. The detachable container 2601 may be used for sample removal, as discussed above. Alternatively, the detachable container 2601 may be used for the insertion of instruments 201 or tubes 1903. When multiple valves 1003 are not needed, a cap 805 with a single valve 1003 or detachable container 2601 may be used. This may be preferable for a surgeon to minimize the points of failure for a breach of the seal and/or to increase the working area for a surgical tool 201.

FIG. 29A shows medical device 2700 with film sleeve 2701 detached from proximal ring 101. Film sleeve 2701 may couple to proximal ring 101 with screw threads 2901 or a similar known temporary coupling method. FIG. 29B shows detachable container 2601 being detached from valve cap 805. Valve cap 805 may have cap screw threads 2903 to attach and detach from film sleeve 2701. Further, detachable container 2601 may have screw threads 2905 to attach and detach from valve cap 805. Alternatively, screw threads 2901, 2903, 2905 may be substituted for other known attachment means.

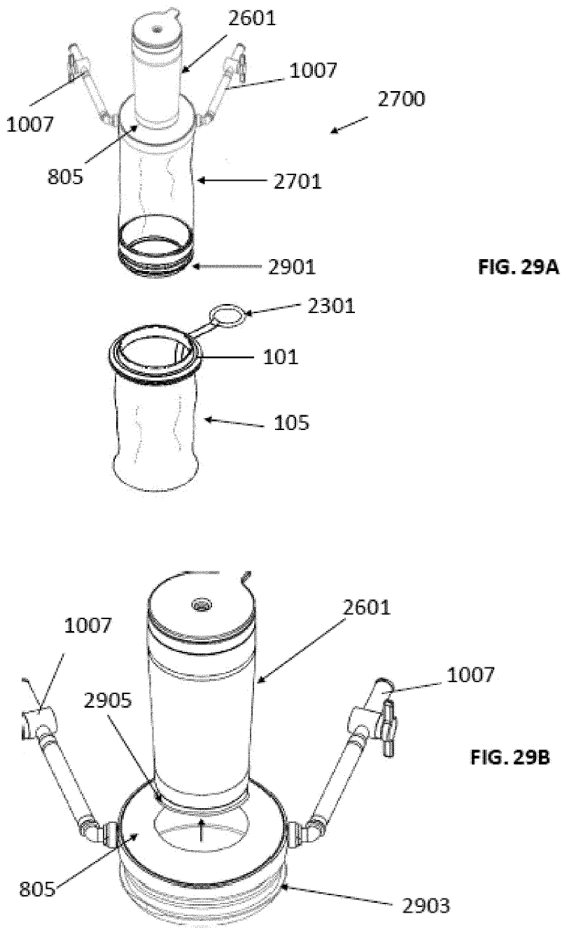

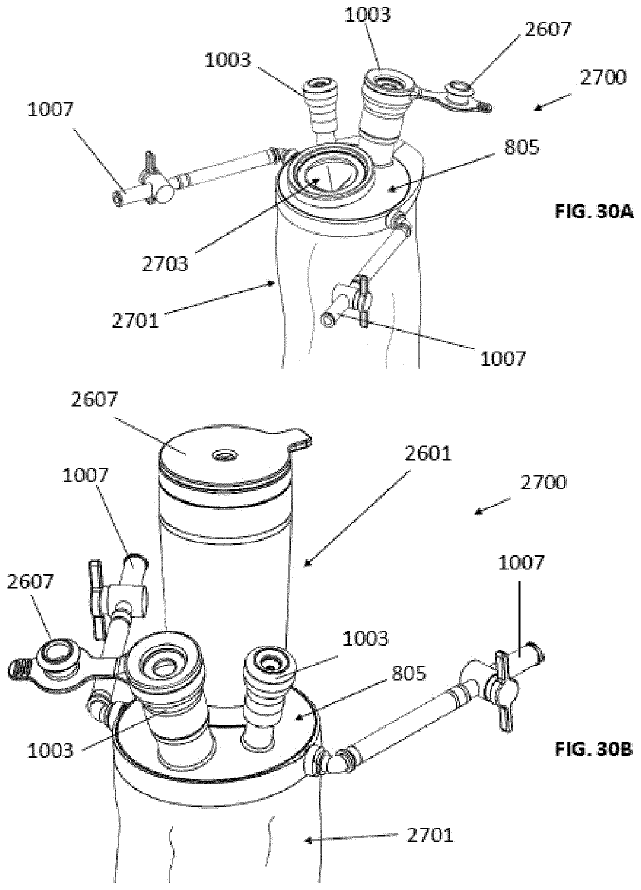

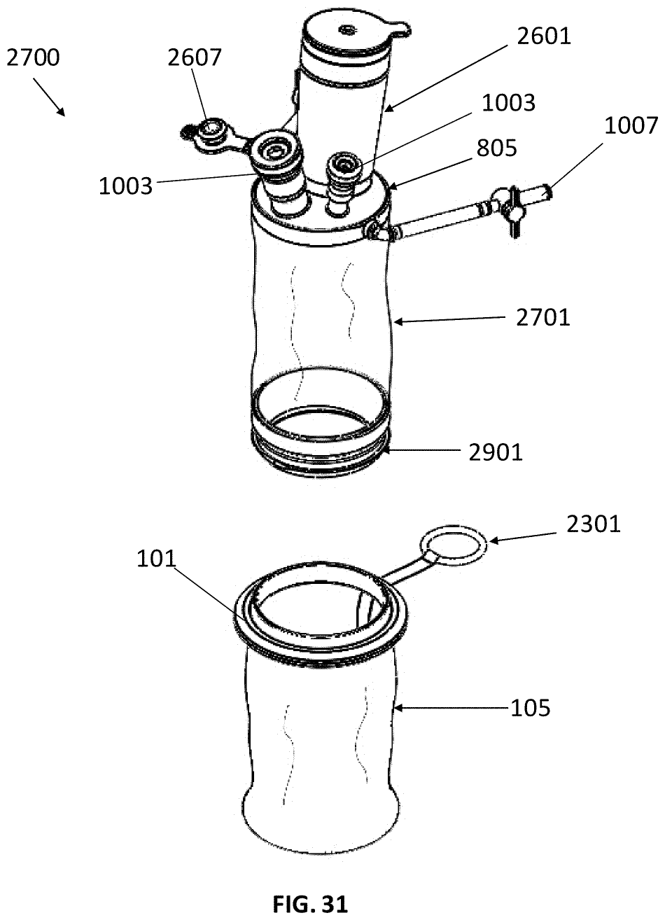

FIGS. 30A-30B show medical device 2700 with the valve cap 805 and detachable container 2601. The cap 805 may have one or more valves 1003 in addition to the detachable container 2601. FIG. 30A shows the detachable container 2601 detached from the valve cap 805. The cap 805 may have an airtight seal 2703 that seals the cap 805 when the detachable container 2601 is detached. FIG. 30B shows the medical device 2700 with the detachable container 2601 attached at the valve cap 805. FIG. 31 shows the medical device 2700 of FIGS. 30A-30B with the film sleeve 2701 detached from the proximal ring 101.

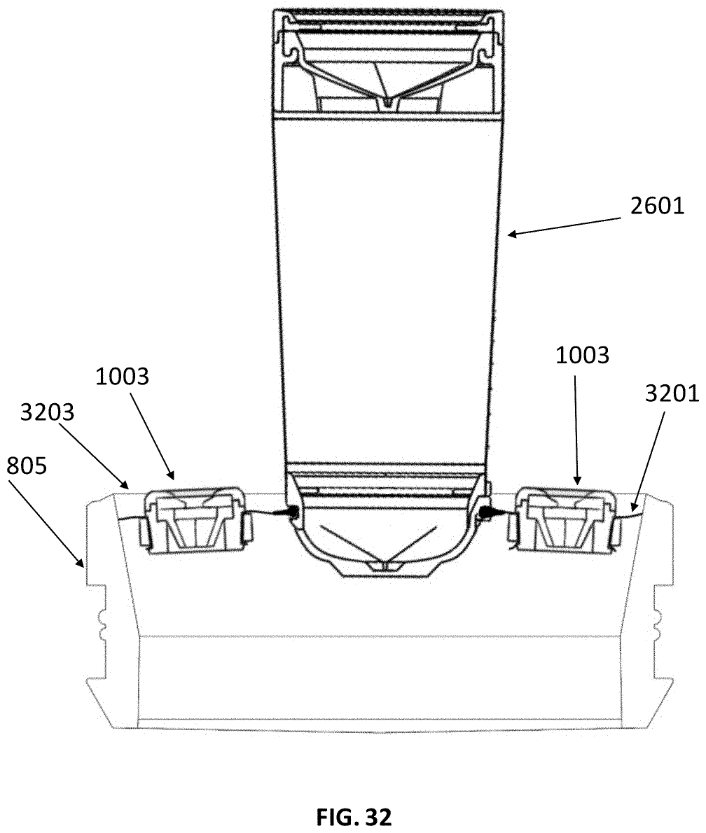

FIG. 32 shows a cap 805 with valves 1003 and detachable container 2601. While the valves 1003 and detachable container 2601 are shown mounted to a cap 805, they may also be mounted to an enclosed proximal end 1001 (e.g., FIG. 10A), a chute 1005 (e.g., FIG. 10B), or elsewise as described herein. The valves 1003 and detachable container 2601 may be mounted onto a flexible film surface 3201 of flexible film 3203. The flexible film surface 3201 may be a semi-rigid or flexible surface capable of movement. The flexible film surface 3201 may allow mounted valves 1003, detachable containers 2601, insufflation ports 1007, or other attachments to have a higher degree of movement than if they were mounted to a rigid surface. This may allow for more freedom of movement for surgical tools 201 being used in a procedure. Flexible film surface 3201 may be contact a proximal side of a valve cap 805. The valves 1003 may extend proximal to flexible film surface 3201. Further, valves 1003 may be located at ports in flexible film surface 3201 that do not substantially extend proximal to cap 805. The flexible film surface 3201 may contact the radial exterior of valves 1003 and then extend distally down the sidewall of valves 1003. Valves 1003 may further contain an outer member that may be used to connect the flexible film surface 3201 to valves 1003. The outer member of valve 1003 may extend radially around the entire circumference of valve 1003. The outer member may contact a distal surface of flexible film surface 3201, while the sidewall of valve 1003 may contact a proximal surface of flexible film surface 3201. The outer member and sidewall of valve 1003 may pinch the flexible film surface 3201 in order to form the valve's 1003 connection with the flexible film surface 3201. The flexible nature of the flexible film surface 3201 may allow for each valve 1003 to move (e.g., rotate) independently of any other valve 1003 connected to cap 805.

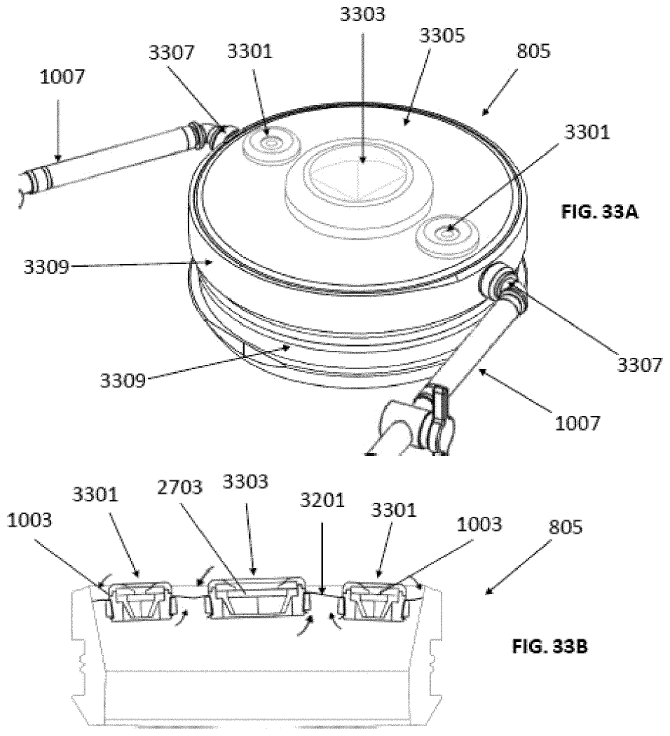

FIG. 33A shows a valve cap 805 that may be mounted at the proximal end of an medical device 100. The valve cap 805 may have one or more valve mounting areas 3301 where extending valves 1003 may be mounted. The mounting area 3301 may be located on the cap surface 3305. Valve mounting areas 3301 may be sealed when a valve 1003 is not mounted. Alternatively, valve mounting areas 3301 may be valves 1003 that do not extend past the cap surface 3305. The cap surface 3305 may be rigid, semi-rigid, or flexible. The cap surface 3305 may be a different material from the rest of the cap 805. The cap surface 3305 may also have one or more detachable container mounting areas 3303 where a detachable container 2601 may be mounted. Alternatively, a tool 201 may be inserted through detachable container mounting area 3303. These mounting areas 3301, 3303 may be sealed when they do not have their respective components mounted.

The cap 805 may include valve connecting areas 3307 on the cap coupling portion 3309 or the cap surface 3305. Cap coupling portion 3309 may be located along the circumference of cap 805. The valve connecting areas 3307 may be where insufflation ports 1007 couple to cap 805. The valve connecting areas 3307 may create an airtight seal with the cap 805. The cap 805 may couple to any access device, sleeve, flexible sleeve, or ring described herein at cap coupling portion 3309. Cap 805 may couple using cap coupling portion 3309 using a friction fit, screw threads, snap features, or similar known coupling means.

FIG. 33B shows a cross sectional view of the valve cap 805 with mounting areas 3301, 3303 described in FIG. 33B. The mounting areas 3301, 3303 may be mounted on a flexible film surface 3201 of the cap 805. This may give a higher degree of movement to the mounting areas 3301, 3303. Each of the mounting areas 3301, 3303 may be able to move independently from the other mounting areas 3301, 3303, the cap 805, and the rest of the device 100. The flexible film 3203 may connect to the valves 1003 as described in FIG. 32.

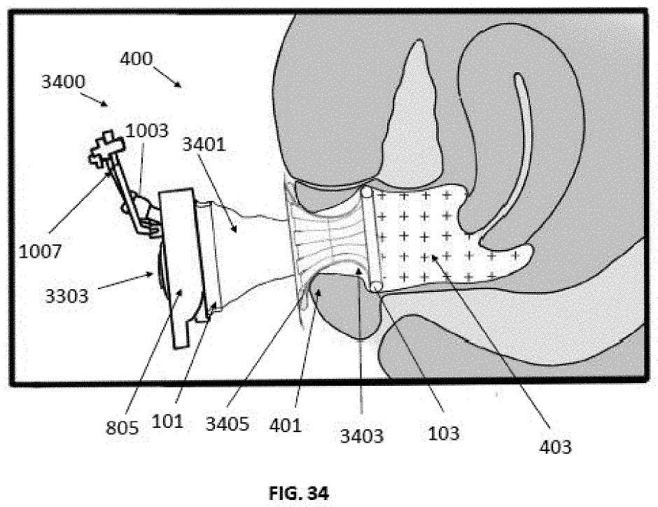

FIG. 34 shows medical device 3400 that includes a valve cap 805 at the proximal ring 101, a film sleeve 3401, a middle ring 3405, a petal portion 3403, and a distal ring 103. The valve cap 805 may include any needed number of detachable sleeve containers 2601, valves 1003, insufflation ports 1007, valve mounting areas 3301, and detachable container mounting areas 3303.

The petal portion 3403 may be positioned between the middle ring 3405 and the distal ring 103. The petal portion 3403 may be a separate component or the same piece as the sleeve 3401. The petal portion 3403 may be a guard that includes multiple members that fan out during use. The plurality of petal members may be movably connected to distal ring 103 and/or the middle ring 3405. The members of the petal portion 3403 may extend from the middle ring 3405 to the distal ring 103. When fanned out, the members may create a lumen 202 through which instruments 201 may access cavity 403. The petal portion 3403 may be inserted into the vagina 400 with the members grouped in a non-fanned out state. After positioning the medical device 3400 within cavity 403, the members of the petal portion 3403 may be fanned out, for example by rolling film sleeve 3401 towards the distal ring 103. Fanning out members of the petal portion 3403 may retract the vaginal wall 401 of the cavity 403.

FIGS. 35A-35D illustrate a medical device 4100 (e.g., a transvaginal device). Medical device 4100 includes a proximal ring 4105, a distal ring 4110, and a membrane 4115 (e.g., a sleeve) extending between proximal ring 4105 and distal ring 4110. Proximal ring 4105 may be an annular ring and may define an lumen 4107. Distal ring 4110 may also be an annular ring and may define an opening 4112. Membrane 4115 may define a lumen 4117 extending between lumen 4107 of proximal ring 4105 and opening 4112 of distal ring 4110. Proximal ring 4105, distal ring 4110, and membrane 4115 may be flexible, semi-rigid, or rigid, and may allow a user to bend or deform these members for insertion into a body opening (e.g., a natural orifice such as a vagina or an incision).

Proximal ring 4105 and/or distal ring 4110 may be integrally formed with membrane 4115. In some instances, membrane 4115 may include an outer wall 4115a, an inner wall 4115b, and a chamber 4115c defined between outer wall 4115a and inner wall 4115b. Chamber 4115c may be inflated with a fluid, e.g., air, via a pump 4140 (FIG. 35D), such as a hand pump or other similar device. Distal ring 4110 may be an extension of membrane 4115 and may also include chamber 4115c. Inflation of chamber 4115c may assist in securing medical device 4100 in a body opening. The combination of proximal ring 4105, distal ring 4110, and membrane 4115 may be referred to as a movable member. The movable member may be configured to move between an insertion position and a radially expanded securing position within an opening as described in greater detail below.

A single tab or leg 4120 (e.g., an elongated member) may extend from proximal ring 4105 into lumen 4117 toward distal ring 4110. Tab 4120 may extend the entire length of lumen 4117 to distal opening 4112 in distal ring 4110, or tab 4120 may terminate proximally of opening 4112. Tab 4120 may include a curve (concave or convex) from a proximal end to a distal end. This curve may assist in conforming medical device 4100 to body opening 4020 when inserted in the patient. In some instances, tab 4120 may be attached to membrane 4115 via an adhesive, laser welding, or the like, which may assist in positioning medical device 4100 in body opening 4020. Alternatively, membrane 4115 may be unattached from tab 4120. Tab 4120 may have a width of approximately 10 mm to approximately 50 mm. The width of tab 4120 may be a distance of tab 4120 perpendicular to longitudinal axis A. Tab 4120 may also include side edges having a curve, and the curve may be approximately 5 degrees to approximately 355 degrees of an equivalent circle. Tab 4120 may be rigid or semi-rigid, and may have a rigidity sufficient to facilitate insertion of membrane 4115 into body opening 4020. The tab 4120 may be biased radially outward. In some instances, a cap 4130 (FIG. 35B) may be attached to a proximal side of proximal ring 4105. Cap 4130 may include one or more access ports 4135 which may receive medical instruments (e.g., medical tools 4150 shown in FIG. 36C) during a medical procedure. It will be understood that the medical instruments may be robotically controlled, manually controlled, or a combination of both. Cap 4135 may be attached to proximal ring 4105 via snap fit or similar connection and may provide a fluid seal between an outside atmosphere and a body cavity. This may assist in maintaining pneumo during a medical procedure.

FIGS. 36A-36F illustrate method of performing a medical procedure using medical device 4100. A user may insert medical device 4100 into a body opening 4020, e.g., a vagina, in an uninflated configuration, as indicated by arrows A in FIG. 36A. Once medical device 4100 is in body opening 4020, a user may inflate membrane 4115 and/or distal ring 4110 using a pump 4140. While shown as a manual pump, it will be understood that pump 4140 may be any pump or device for supplying a fluid to inflate, or removing a fluid to deflate, a device. As described above, inflating membrane 4115 and/or distal ring 4110 may increase a size (e.g., a diameter) of lumen 4107. The user may then attach cap 4130 to proximal ring 4105, shown in FIG. 36C. Medical tools 4150 may be subsequently inserted into a body cavity B via ports 4135 and lumen 4107 to perform a medical procedure.

In another example, cap 4130 may be attached to proximal ring 4105 prior to inserting medical device 4100 into body opening 4020, as shown in FIG. 36D. Additionally, medical tools 4150 may extend through ports 4135 and into lumen 4107 during insertion of medical device 4100 into body opening 4020. A user may push on a proximal end of medical device 4100 to seat medical device 4100 within body opening 4020. Subsequently, the user may inflate membrane 4115 and/or distal ring 4110 using pump 4140. Once the medical procedure is complete, the user may deflate membrane 4115 and/or distal ring 4110 to release the inflating fluid. The user may then pull on the proximal end of medical device 4100 to remove medical device 4100 from body opening 4020.

FIGS. 37A-37C illustrate a medical device 4100′ according to another example. Medical device 4100′ may be similar to medical device 4100 in FIGS. 35A-35D. Medical device 4100′ may include proximal ring 4105, distal ring 4110, and membrane 4115. Medical device 4100′ may also include two tabs 4120. Tabs 4120′ may be similar to tab 4120 in FIGS. 35A-35D. In some instances, tabs 4120′ may be of different length and/or of different materials from each other. Tabs 4120′ may be spaced on radially opposite sides of medical device 4100′, or may be positioned at any other location around proximal ring 4105. Inflation of distal ring 4110 and/or chamber 4115c may cause medical device 4100′ to expand radially outward as indicated by arrow C in FIG. 37C. As shown in FIGS. 37A and 3B, an opening in proximal ring 4105 may be different to be used with various sized body openings. Medical device 4100′ may be inserted into body opening 4020 in a similar manner as described relative to medical device 4100 in FIGS. 36A-36F.

A medical device 4200 is shown in FIGS. 38A and 38B. Medical device 4200 may be similar to medical devices 4100, 4100′ and may include a proximal ring 4205 having an opening, a distal ring 4210 having an opening, and a membrane 4215 connecting distal ring 4210 to proximal ring 4205. A lumen may be defined by membrane 4215 and may connect the opening in proximal ring 4205 to the opening in distal ring 4210. Similar to medical device 4100′, two tabs 4220 may extend from proximal ring 4205 into the lumen defined by membrane 4215. It will be understood that membrane 4215 and/or distal ring 4210 may be inflatable. Membrane 4215 may be similar to membrane 4115, and may include an outer membrane 4215a, an inner membrane 2515b, and a chamber 4215c defined between outer membrane 4215a and inner membrane 4215b.

Proximal ring 4205 may be expendable. For example, proximal ring 4205 may expand from a generally ring shaped member in FIG. 38A to a generally elliptical shaped member in FIG. 38B. A force on proximal ring 4205 radially outward in the direction indicated by arrows in FIG. 38B may allow proximal ring 4205 to expand by sliding along connectors 4205a, 4205b. Expansion of proximal ring 4205 may assist in increasing the body opening.

A method of using medical device 4200 is shown in FIGS. 39A-39B. Medical device 4200 may be inserted into body opening 4020 as shown in FIG. 39A. A user may expand proximal ring 4205 in a radially outward direction, as indicated by arrows D. Proximal ring 4205 may include a ratchet device or similar friction device that may maintain the expanded configuration. Cap 4130 may be flexible and/or may include different shapes and/or sizes, which may allow cap 4130 to conform to different shapes and/or size of proximal ring 4205 to create a fluid seal. In some instances, pump 4140 may be used to inflate membrane 4215 and/or distal ring 4210. A cap 4130 may be attached to proximal ring 4205. It will be understood that cap 4130 may have a shape similar to the shape of expanded proximal ring 4205. One or more medical instruments may be inserted into body cavity B via ports in cap 4130 and the lumen defined by membrane 4215. One or more of deflation of membrane 4215 and/or distal ring 4210, and a distal force on medical device 4200 may be used to remove medical device 4200 from body opening 4020.

A medical device 4200′ is shown in FIGS. 40A-40C. Medical device 4200′ may be similar to medical device 4200 in FIGS. 38A and 38B. Unlike medical device 4200, medical device 4200′ may include a proximal ring 4205′ that does not expand. In some instances, proximal ring 4205′ may also be rigid, e.g., may not bend. In other instances, proximal ring 4205′ may include a material capable of bending. Medical device 4200′ may include one, two, or more tabs or legs 4220 as shown in FIG. 40B and/or inflatable portions, e.g., membrane 4215 and/or distal ring 4210, shown in FIG. 40C. As described above, membrane 4215 may be attached or may be unattached to tabs 4220.

A method of inserting medical device 4200′ in body opening 4020 is shown in FIGS. 41A-41C. A fore in a proximal direction may move medical device 4200′ into opening 4020 and towards body cavity B. A user may inflate one or more portions of medical device 4200′ as described herein, e.g., using pump 4140. In some instances, cap 4130 may be attached to proximal ring 4205′ to provide a fluid seal between the outer atmosphere and body cavity B. Medical device 4200′ may be removed from body opening 4020 in any manner described herein.

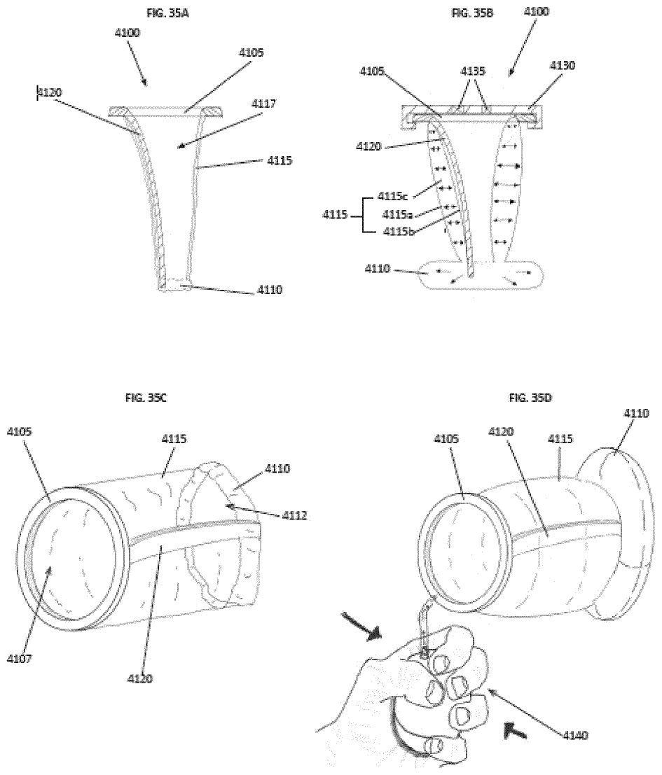

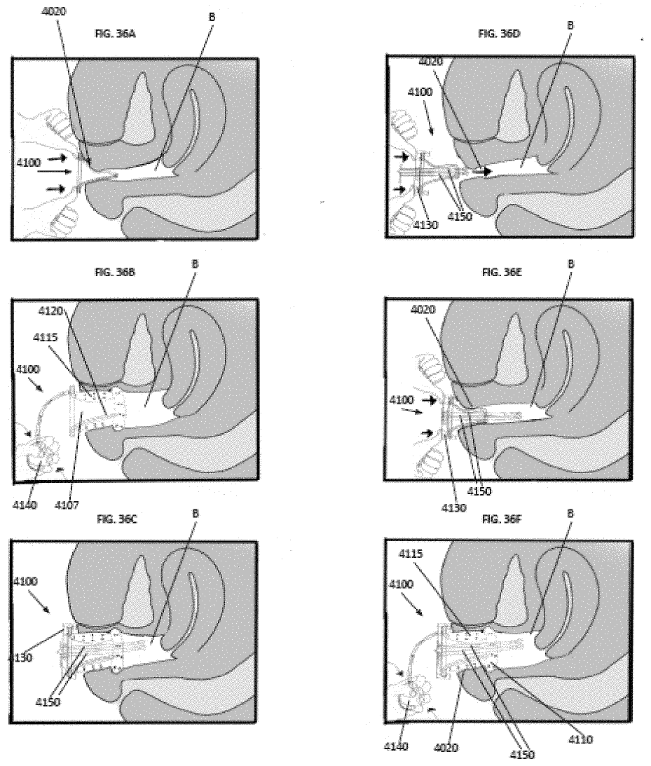

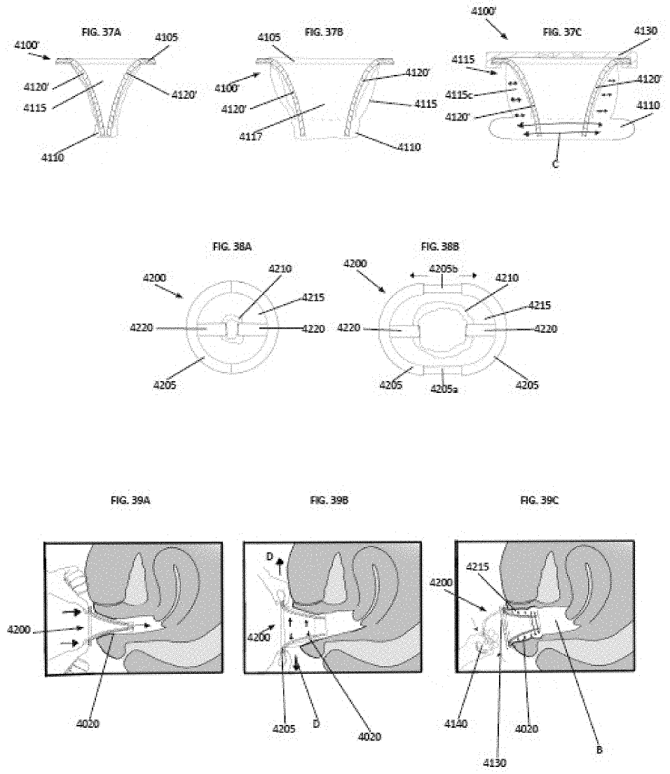

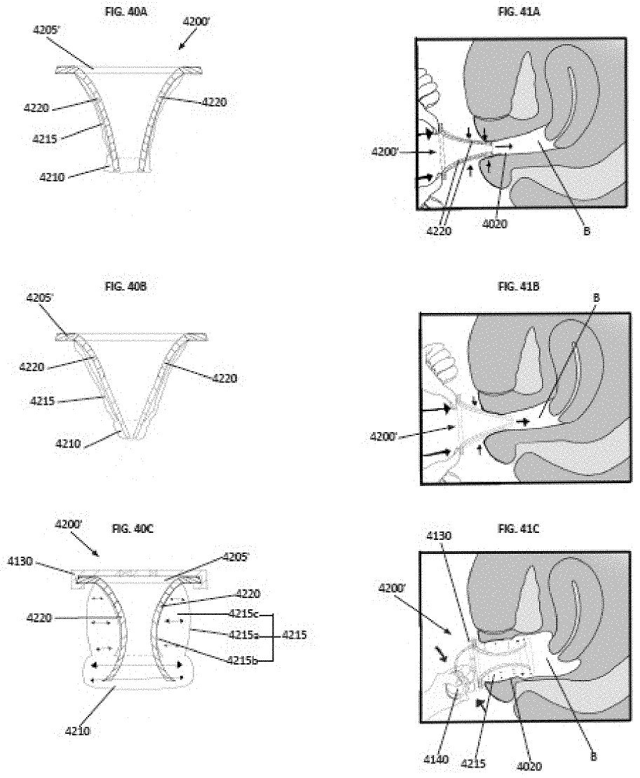

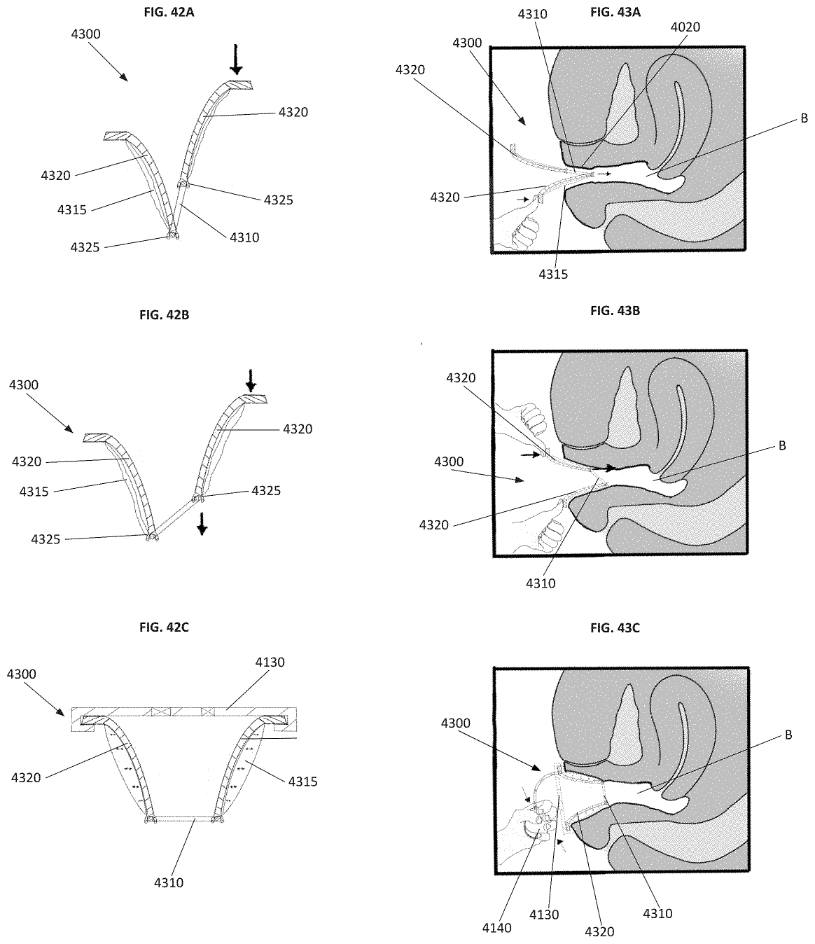

A medical device 4200 is shown in FIGS. 42A-42C. Medical device 4300 may include two or more tabs or legs 4320. Tabs 4320 may be connected at their distal ends via a distal ring 4310. Each tab 4320 may be attached via a pivot connection 4325 to distal ring 4310. In some instance, a membrane 4315 extends from distal ring 4310 to a proximal end of tabs 4320 and may be connected to tabs 4320, e.g., via adhesive, laser welding, or the like. Membrane 4315 may define a lumen in which tabs 4320 may be disposed. Membrane 4315 may be inflatable. Further, membrane 4315 may be connected to a radially outer surface of the tabs 4320 and a central lumen may be defined by a radially inner surface of the tabs 4320.

Pivot connections 4325 may allow medical device 4300 to be inserted into a body opening 4020 in a generally linear configuration (FIG. 42A) where distal ring 4310 is near to parallel with tabs 4320. Tabs 4320 may pivot relative to distal ring 4310 via pivot connections 4325 and may allow medical device 4300 to expand such that distal ring 4310 is generally perpendicular to tabs 4320, as shown in FIG. 42C. In some instances, cap 4130 may be attached to a proximal end of tabs 4320 to secure the pivoting position of tabs 4320 and provide a fluid seal between the outside atmosphere and a body cavity.

A method of inserting medical device 4300 into body opening 4020 is shown in FIGS. 43A-43C. Medical device 4300 may be inserted in a generally parallel orientation shown in FIG. 43A. In some instances, a first one of tabs 4320 may be inserted into opening 4020. The second one of tabs 4320 may be subsequently inserted into opening 4020 in FIG. 43B. As the second tab 4320 is moved distally, distal ring 4310 pivots relative to tabs 4320 and achieves a generally perpendicular orientation relative to tabs 4320. Membrane 4315 may be inflated to further secure medical device 4300 within body opening 4020. Medical instruments may be inserted through the lumen of medical device 4300 and into body cavity B. Medical device 4300 may be removed from body opening 4020 in any manner described herein.

A medical device 4300′ is shown in FIGS. 44A-44C. Medical device 4300′ may be similar to medical device 4300 in FIGS. 42A-42C, and may include distal ring 4310 pivotally connected to distal end of tabs 4320 via pivot connections 4325. Medical device 4300′ may also include a proximal ring 4305′ and which may be pivotally connected to proximal ends of tabs 4320 via pivot connections 4313. Proximal ring 4305′ may be a non-expandable member, or may expand from a generally ring shaped member to an elliptical shape, shown in FIG. 44D and similar to proximal ring 4205 in FIG. 38B. As with medical device 4300, medical device 4300′ may move from a generally parallel configuration in FIG. 44A to a configuration in which proximal ring 4305′ and distal ring 4310 are generally perpendicular to tabs 4320. Membrane 4315 may connect proximal ring 4305′ to distal ring 4310, which may define a lumen therebetween.

A method of inserting medical device 4300′ is shown in FIGS. 45A-45C. Medical device 4300′ may be inserted into a body opening 4020 in a generally parallel configuration in which distal ring 4310, tabs 4320, and proximal ring 4305′ are generally parallel to each other. In this instance, one tab 4320 may be inserted into opening 4020 prior to insertion of the second tab 4320.

As shown in FIG. 45B, the second tab 4320 may be moved in a distal direction and may cause distal ring 4310 to pivot about pivot connections 4325 and proximal ring 4305′ to rotate about pivot connections 4313. This may cause distal ring 4310 and proximal ring 4305′ to be generally perpendicular to tabs 4320, as shown in FIG. 45C. Additionally, proximal ring 4305′ may be moved radially outward as indicated by arrows E in FIG. 45B. This may assist in securing medical device 4300′ in body opening 4020 and/or may increase the size of the lumen defined by membrane 4315. In some instances, cap 4130 may be attached to proximal ring 4305′ which may assist in creating a fluid seal between the outside atmosphere and body cavity B. Additionally, or alternatively, pump 4140 may be used to inflate membrane 4315 which may assist in securing medical device 4300′ in body opening 4020. After the medical procedure is performed, medical device 4300′ may be removed from body opening 4020 in any manner described herein.

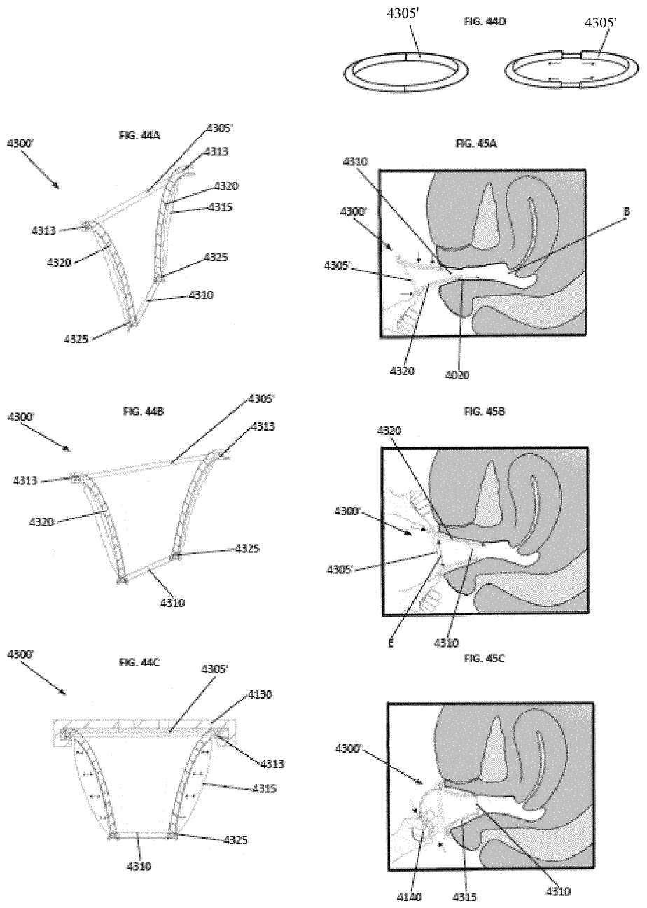

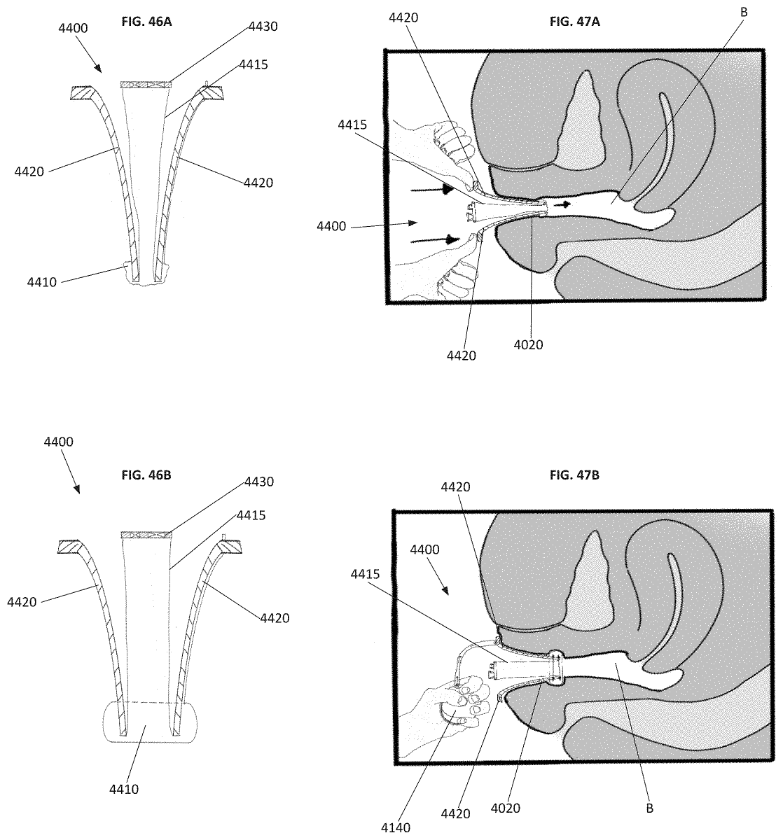

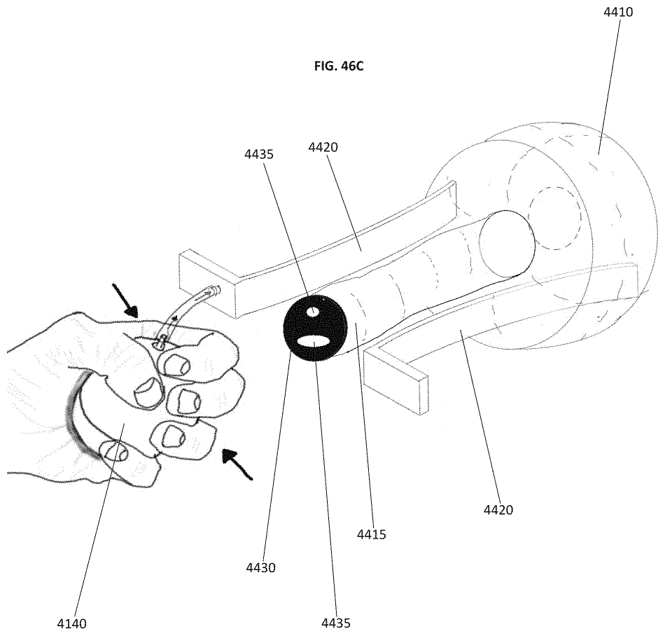

A medical device 4400 is shown in FIGS. 46A-46C. Medical device 4400 includes two tabs or legs 4420. A membrane 4415 may be attached to distal ends of tabs 4420 and a lumen may be formed by a wall of membrane 4415. A distal portion of membrane 4415 may form a distal ring 4410. Alternatively, distal ring 4410 may be separately formed and attached to the distal end of tabs 4420 and/or a distal end of membrane 4415. A cap 4430 may be attached to a proximal end membrane 4415. Capp 4430 may include one or more lumens 4435 capable of receiving medical instruments. Tabs 4420 may be positioned radially outward of membrane 4415. Pump 4140 may be attached to medical device 4400 to inflate one or more chambers defined by membrane 4415 and/or distal ring 4410. In some instances, membrane 4415 includes a flexible material that may allow a user additional movement of medical tools extending into the body via body opening 4020.

FIGS. 47A and 47B illustrate a method of inserting medical device 4400 into body opening 4020. A user may insert distal ends of tabs 4420 into body opening 4020 and move medical device 4400 distally into body cavity B. Subsequent inflation of distal ring 4410 may secure medical device 4400 in body opening 4020. Medical device 4400 may be removed from body opening 4020 in any manner described herein.

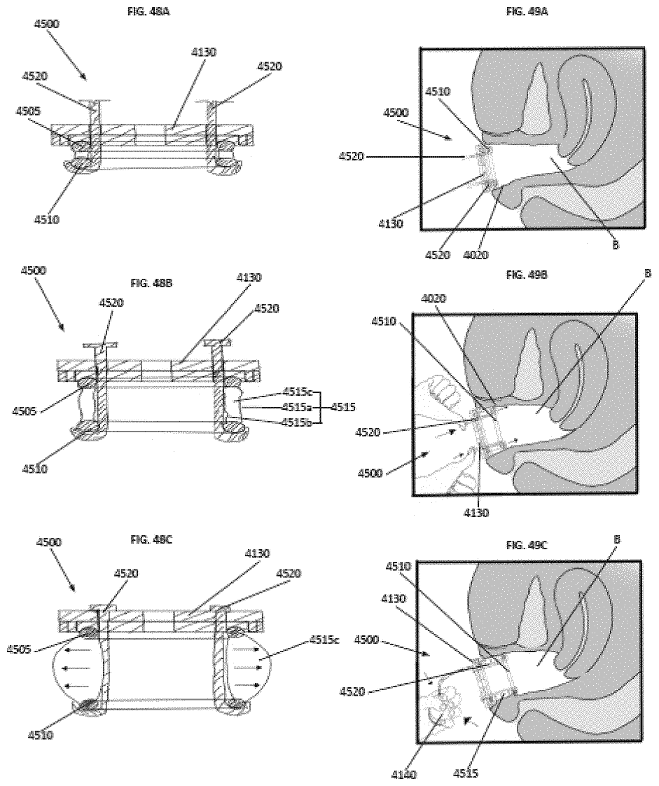

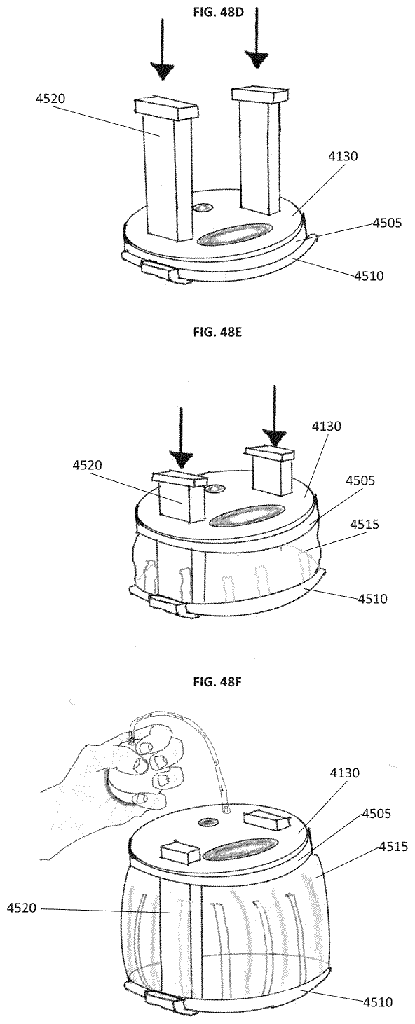

A medical device 4500 according to another example is shown in FIGS. 48A-48F. Medical device 4500 may include a proximal ring 4505, a distal ring 4510, and a pair of tabs or legs 4420 attached to distal ring 4510. A membrane 4515 may be attached at a proximal end to proximal ring 4505 and at a distal end to distal ring 4510. Cap 4130 may be attached to proximal ring 4505. Tabs 4520 may be connected to one or both of cap 4130 or proximal ring 4505. Tabs 4520 may move proximally and distally relative to cap 4130 and proximal ring 4505. For example, proximal ring 4505 and distal ring 4510 may be positioned adjacent each other in an unexpanded configuration in FIG. 48A. Distal movement of tabs 4520 may cause distal ring 4510 to move distally to an expanded configuration (FIG. 48C). Movement from the unexpanded configuration to the expanded configuration may cause membrane 4515 to expand in a proximal-distal direction. Membrane 4515 may be similar to membrane 4115, and may include an outer membrane 4515a, an inner membrane 4515b, and a chamber 4515c defined between outer membrane 4515a and inner membrane 4515b. Pump 4140 may be connected to medical device 4500 and may inflate and/or deflate chamber 4515c.

A method of performing a medical procedure is shown in FIGS. 49A-49C. Medical device 4500 may be inserted into body opening 4020 in the unexpanded configuration. Pushing on proximal ends of tabs 4520 in the distal direction may cause distal ring 4510 to move distally relative to proximal ring 4505. Membrane 4515 may subsequently be inflated and may secure medical device 4500 in body opening 4020. To remove medical device 4500, membrane 4515 may be deflated and medical device 4500 may be moved in a proximal direction from body opening 4020.

FIGS. 50A-50F illustrate a medical device 4600. Medical device 4600 include a proximal ring 4605 and a plurality (e.g., two, three, four, or more) of tabs or legs 4620 extending distally from proximal ring 4605. Tabs 4620 may be biased radially outward by a material and/or a shape of tabs 4620. A distal ring 4610 may be attached to one or more actuators 4612. Distal ring 4610 may be positioned at a distal end of tabs 4620 in an unexpanded configuration and may prevent tabs 4620 from expanding. Actuator 4612 may be moved proximally and may move distal ring 4610 in a proximal direction. As distal ring 4610 moves proximally, tabs 4620 may expand radially outward to their natural biased position.

Actuators 4612 may extend through openings in cap 4130 to allow a user to grasp and move actuators 4612 in the proximal direction. In some instances, a membrane 4615 may be attached at a distal end to a distal end of each of tabs 4620. A proximal end of membrane 4615 may be attached to distal ring 4610. Membrane 4615 may be similar to other membranes described herein and may define a chamber that may be inflated. As distal ring 4610 is moved in the proximal direction, membrane 4615 may form a lumen between proximal ring 4605 and distal ends of tabs 4620. The lumen may provide access to a body cavity B (e.g., FIG. 51A), as described herein. Alternatively, an inner surface of distal ring 4610 may be disposed radially outward of tabs 4620 and membrane 4615. In this instance, membrane 4615 may be fixed to proximal ring 4605 and the distal end of each of tabs 4620. Movement of distal ring 4610 in the proximal direction in this example may cause medical device 4600 to transition from the unexpanded configuration to the expanded configuration.

A method of performing a medical procedure using medical device 4600 is shown in FIGS. 51A-51C. Medical device 4600 may be inserted into body opening 4020 by pushing on proximal ends of tabs 4620, which may position medical device 4600 in body opening 4020. Actuators 4612 may then be moved in a proximal direction, which may cause distal ring 4610 to move in the proximal direction. Proximal movement of distal ring 4610 may allow tabs 4620 to move radially outward to their natural bias. The proximal movement of distal ring 4610 may also cause membrane 4615 to create a lumen between openings in proximal ring 4605 and distal ring 4610. The user may also inflate chambers formed within membrane 4615 using pump 4140. The radial expansion of tabs 4620 and/or the inflation of membrane 4615 may secure medical device 4600 in body opening 4020. Medical device 4600 may be removed in any manner described herein. For example, distal ring 4610 may be moved distally to move tabs 4620 radially inward. Alternatively, or additionally, a proximal force on medical device 4600 may remove medical device 4600 from body opening 4020.

A medical device 4700 according to another example is shown in FIGS. 52A-52D. Medical device 4700 may include a ratchet-mechanism to move from an unexpanded configuration to an expanded configuration. A pair of crossbars 4705 may each include a plurality of protrusions 4705a. Two movable members 4707 may each have openings 4707a and 4707b. Openings 4707a may receive a first crossbar 4705 and openings 4707b may receive a second crossbar 4705. Movable members may also be pivotally connected at two pivot points 4709. A distal ring 4710 may be attached to distal ends of movable members 4707. A membrane 4715 may surround a radially outer surface of movable members 4707. In some instances, membrane 4715 may include an inflatable cavity.

Movable members 4707 may move from an unexpanded configuration in FIG. 52A to an expanded configuration in FIG. 52B. As openings 4707a and 4707b move relative to crossbars 4705, openings 4707a and 4707b may engage protrusions 4705a. The cooperation between openings 4707a and 4707b and protrusions 4705a may maintain an expanded configuration. To move medical device 4700 from the expanded configuration to the unexpanded configuration, it may be necessary to supply a force sufficient to overcome the holding force of the protrusions. Alternatively, or additionally, a release mechanism may be provided on openings 4707a and 4707b to allow medical device 4700 to move to the unexpanded configuration.

As with other example medical devices described herein, membrane 4715 and/or distal ring 4710 may be expanded to further secure medical device 4700 within body opening 4020.

A medical procedure is described with reference to FIGS. 53A-53C. Medical device 4700 may be inserted into 4020 body opening in FIG. 53A. Movable members 4707 may be moved radially outward via pivot points 4709. The expansion of medical device 4700 may cause openings 4707a, 4707b to cooperate with protrusions 4705a and may prevent medical device 4700 from collapsing to the unexpanded configuration.

After medical device 4700 is expanded, membrane 4715 may be inflated. Medical instruments may be inserted through the lumen defined by membrane 4715. Once the medical procedure is complete, membrane 4715 may be deflated and a force sufficient to move movable members 4707 into the unexpanded configuration may be supplied to medical device 4700.

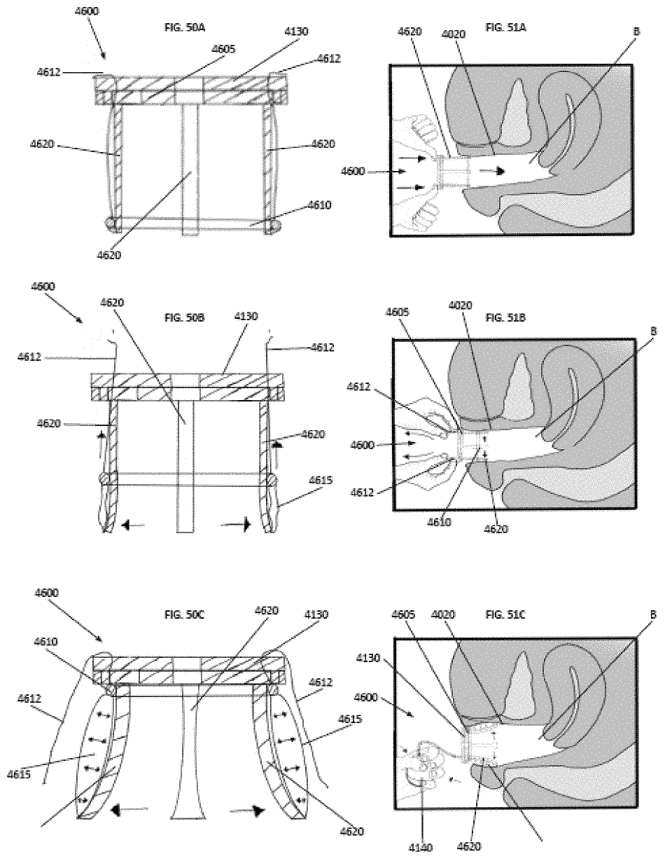

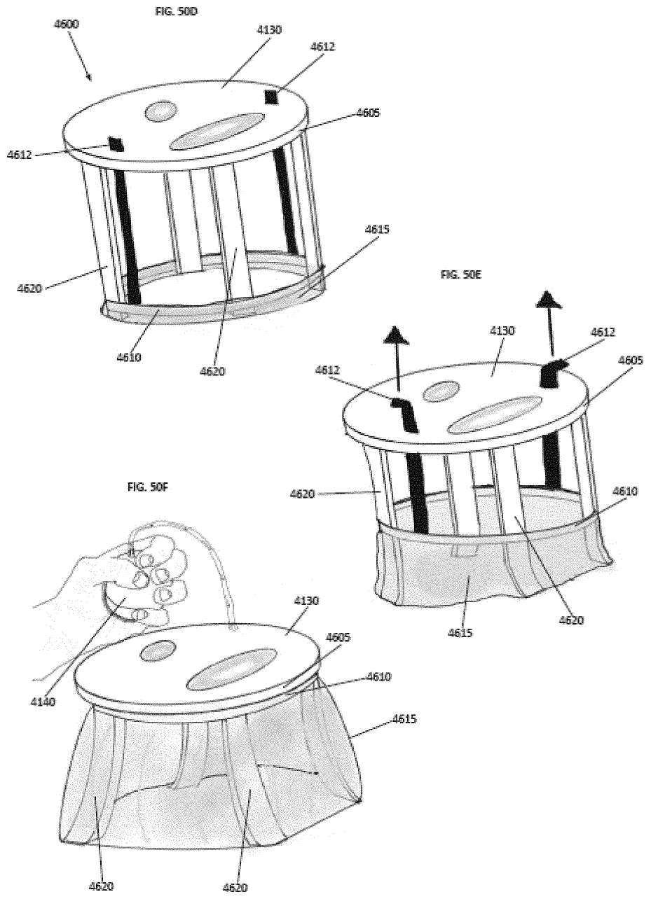

A medical device 4800 according to another example is shown in FIGS. 54A-54D. Medical device 4800 may include an annular member 4805 (FIG. 54B) with a cap 4830 attached to a proximal end. Cap 4830 may be similar to cap 4130. Annular member 4805 may be rigid or semi-rigid and may define a lumen 4807. Cap 4830 may include one or more ports 4835 to access lumen 4807. An expandable member 4815 may be attached to a radially outer surface of annular member 4805. Expandable member 4815 may include one or more expandable cavities which may be inflated together or separately. Expandable member 4815 may create a plurality of rings when inflated, which may assist in securing medical device 4800 in body opening 4020. Expandable member 4815 may include a plurality of ring-shaped members coaxial with a central axis of the lumen. Each of the plurality of ring-shaped members may be independently inflatable.

A medical procedure using medical device 4800 is described with reference to FIGS. 55A-55C. Medical device 4800 may be inserted into body opening 4020 by pushing on a proximal end of medical device 4800, as shown in FIG. 55A. Once seated in body opening 4020, expandable member 4815 may be inflated using pump 4140. Medical tools may be inserted via ports 4835 into lumen 4807. After the medical procedure is complete, expandable member 4815 may be deflated and medical device 4800 may be removed from body opening 4020.

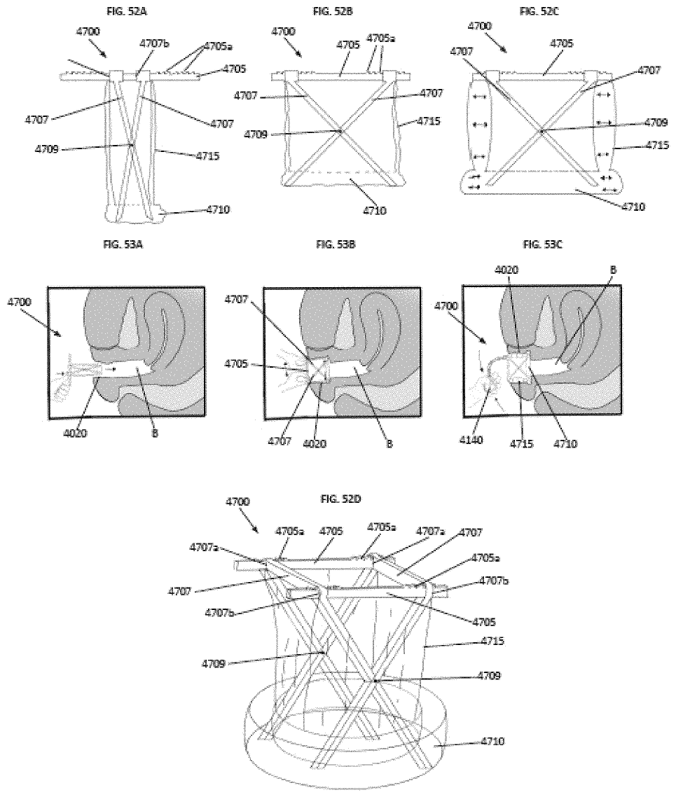

A medical device 4800′ is described with reference to FIGS. 56A-56D. Medical device 4800′ may be similar to medical device 4800 in FIGS. 54A-54D. Medical device 4800′ may include an annular member 4805. Annular member 4805 may be rigid or semi-rigid and may define a lumen 4807. An expandable member 4815 may be attached to a radially outer surface of annular member 4805. Expandable member 4815 may include one or more expandable cavities which may be inflated together or separately. Expandable member 4815 may create a plurality of rings when inflated, which may assist in securing medical device 4800 in body opening 4020.

A membrane 4820′ may extend within lumen 4807 from a distal end of annular member 4805 proximal of a proximal end of annular member 4805. Membrane 4820′ may define a lumen 4820a′ that extends from a proximal end to a distal end thereof. A cap 4830′ may seal a proximal end of lumen 4820a′ of membrane 4820′. Cap 4830′ may include one or more ports 4835′ to access lumen 4820a′. Membrane 4820′ may be flexible and may allow improved movement of medical instruments extending through ports 4835′ during a procedure.

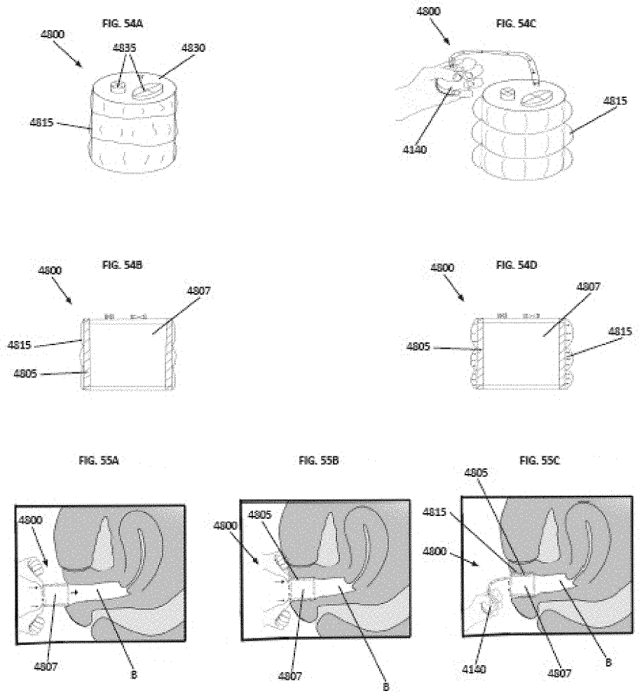

A medical procedure using medical device 4800′ is described with reference to FIGS. 57A-57C. Medical device 4800′ may be inserted into body opening 4020 in a similar manner as that described for inserting medical device 4800′ in FIGS. 55A-55C. During the medical procedure, tools may be inserted via ports 4835′. The flexibility of membrane 4820′ may allow the medical instruments to have greater movement relative to medical device 4800′, which may improve one or more aspects of the medical procedure, e.g., by enabling the medical instruments to access additional tissue.

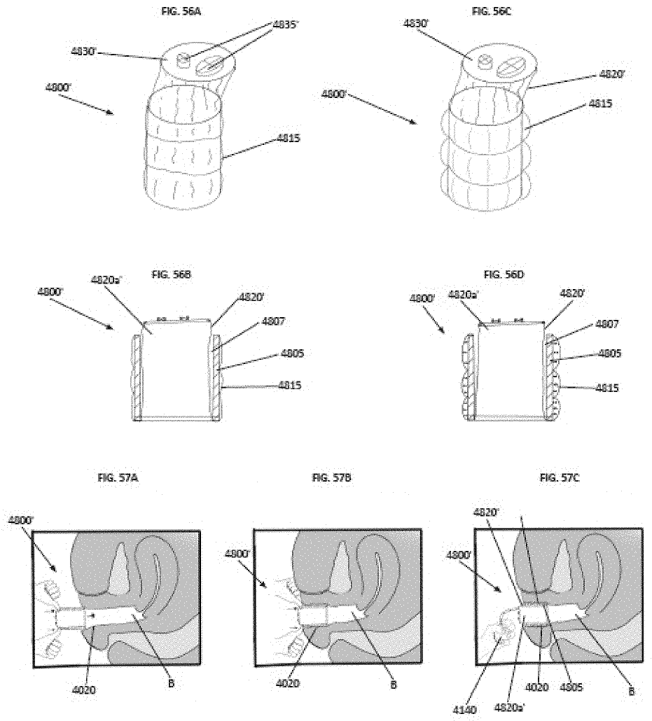

A medical device 4900 is described with reference to FIGS. 58A-58C. Medical device 4900 includes an annular member 4905. Annular member 4905 may include a distal ring 4910 connected to a distal end thereof. Annular member 4905 may expand radially outward as indicated by arrows F to have a generally elliptical shape, as shown in FIG. 58B. A cap 4130 may fluidly seal a proximal end of annular member 4905. Pump 4140 may inflate or deflate distal ring 4910. Expansion of annular member 4905 to the elliptical shape and/or inflation of distal ring 4910 may assist in securing medical device 4900 in a body opening. Medical device 4900 may be inserted into a body opening in a manner similar to that described in FIGS. 39A-39C relative to medical device 4200.

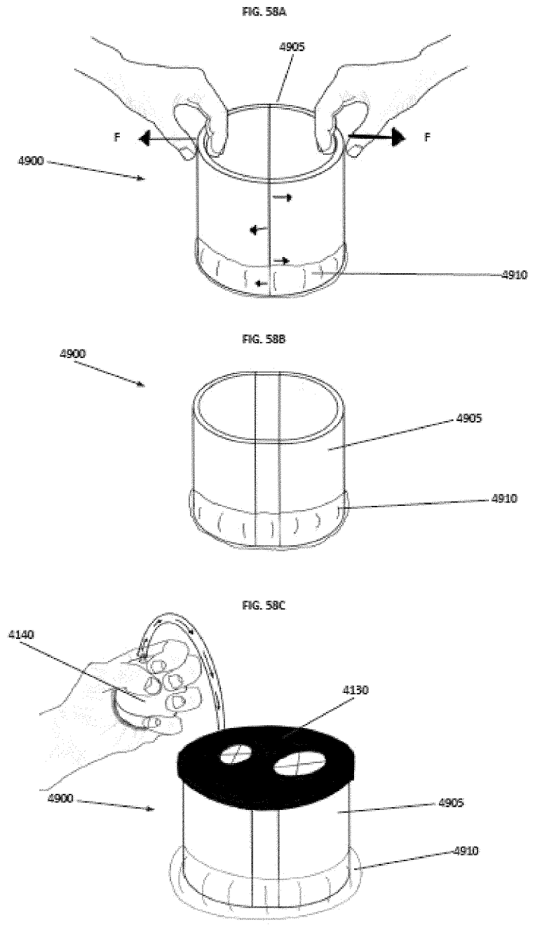

A medical device 5000 is illustrated with reference to FIGS. 59A and 59B. Medical device 5000 may be formed of a material configured to expand radially outward in a direction indicated by arrows G when proximal and distal ends of medical device 5000 are rotated about an axis of medical device 5000. For example, a proximal end of medical device 5000 may be rotated in a counterclockwise direction as indicated by arrow H, while a distal end of medical device 5000 is simultaneously rotated in a clockwise direction as indicated by arrow J. This rotational movement of medical device 5000 may cause medical device 5000 to change shape from a generally cylindrical shape to a configuration including a bulge expanding radially outward, as shown in FIG. 59B. Rotational movement may also cause a height of medical device 5000 to decrease, which may allow a portion of medical device 5000 to expand radially outward. The bulge of medical device 5000 may assist in securing medical device 5000 in a body opening. While not shown, a cap (e.g., cap 4130 in FIG. 35B) may be attached to a proximal end of medical device 5000 to fluidly seal a lumen extending therethrough.

Accordingly to an example, medical device 5000 may be inserted into a body opening in the cylindrical configuration of FIG. 59A. The proximal end of medical device 5000 may be rotated relative to the distal end of medical device 5000. In some instances, a tool may be inserted through the lumen of medical device 5000 to maintain a position relative to, and/or rotate the distal end in a direction opposite of, the proximal end of medical device 5000. Once the medical procedure is complete, medical device 5000 may be rotated in an opposite direction to cause the bulge to move radially inward and move medical device 5000 to the cylindrical shape.

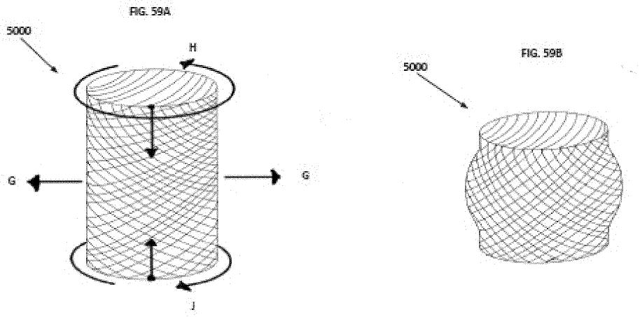

A medical device 5100 according to another example is shown in FIGS. 60A-60D. An annular member 5105 may define a lumen 5109 and may include multiple chambers 5107 which may be inflated or deflated. For example, annular member 5105 may include an outer wall 5105a and an inner wall 5105b. Portions of outer wall 5105a may be attached to portions of inner wall 5105b at multiple locations (e.g., via laser welding, adhesive, etc.) to create chambers 5107. Chambers 5107 may be coaxially arranged. These multiple locations may create reinforcement members 5105c, which may have increased rigidity relative to other portions of annular member 5105.

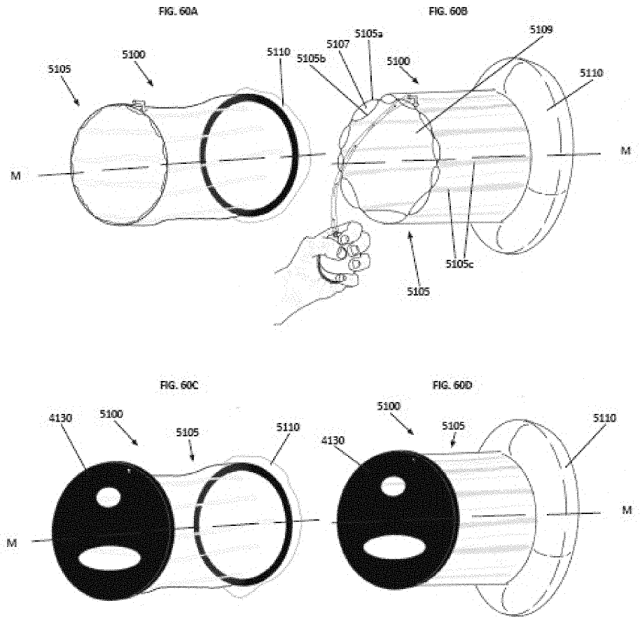

A distal ring 5110 may be attached to a distal end of annular member 5105, and cap 4130 (or other similar cap) may seal a proximal end of lumen 5109 defined by annular member 5105. Distal ring 5110 may be an inflatable member, as shown in FIGS. 60B and 60D, or may be incapable of being inflated, as shown in FIGS. 60A and 60C. In some instances, a cap 4130 may be attached to a proximal opening of lumen 5109, as shown in FIG. 61C.

Medical device 5100 may move from an uninflated configuration to an inflated configuration. For example, chambers 5107 may be uninflated in FIG. 60A, and may be inflated by pump 4140 in FIG. 60B. Inflating annular member 5105 may orient distal ring 5110 in a direction perpendicular to a longitudinal axis M of medical device 5100. For example, as chambers 5107 are inflated, reinforcement members 5105c may become aligned parallel to longitudinal axis M. This alignment may cause distal ring 5110, connected to the distal end of annular member 5105, to become generally perpendicular to longitudinal axis M. In some instances, distal ring 5110 may also be inflated, which may assist in orienting distal ring 5110 generally perpendicular to longitudinal axis M.

A medical procedure using medical device 5100 according to an example is shown in FIGS. 61A-61C. Distal ring 5110 of medical device 5100 may be positioned sideways, parallel to longitudinal axis M, and may be inserted into body opening 4020 in this manner. In other words, a cross-sectional area of distal ring 5110 may be minimized such that distal ring 5110 may be inserted into body opening 4020. As chambers 5107 and/or distal ring 5110 of medical device 5100 are inflated, lumen 5109 may expand. Medical instruments may be advanced into body cavity B via ports in cap 4130 and/or lumen 5109. Deflation of chambers 5107 and/or distal ring 5110 may allow medical device 5100 to be removed from the body.

Once disposed within body opening 4020, the user may inflate chambers 5107 and/or distal ring 5110 using pump 4140, which may reorient distal ring 5110 to be perpendicular to longitudinal axis M. For example, as chambers 5107 are filled with fluid (e.g., air), reinforcement members 5105c may extend to be parallel to longitudinal axis M. The general alignment of reinforcement members 5105c to be parallel with longitudinal axis M may cause distal ring 5110 to become perpendicular to longitudinal axis M. In the event distal ring 5110 includes an inflatable chamber, distal ring 5110 may be inflated subsequent to, or concurrently with, chambers 5107.

A medical device 5200 according to another example is shown with reference to FIGS. 62A-62C. Medical device 5200 may be bell shaped, having a smaller outer diameter at a proximal end and a larger outer diameter at a distal end. A proximal ring 5205 may be attached to a proximal end of an inflatable body 5210. While not shown, a distal ring may be attached to a distal end of inflatable body 5210. Inflatable body 5210 may include one or more chambers 5210a which may be inflated using a pump (not shown). One or more lumens 5215 may be defined by inflatable body 5210 and may extend from an opening in proximal ring 5205 to an opening in the distal end of inflatable body 5210.

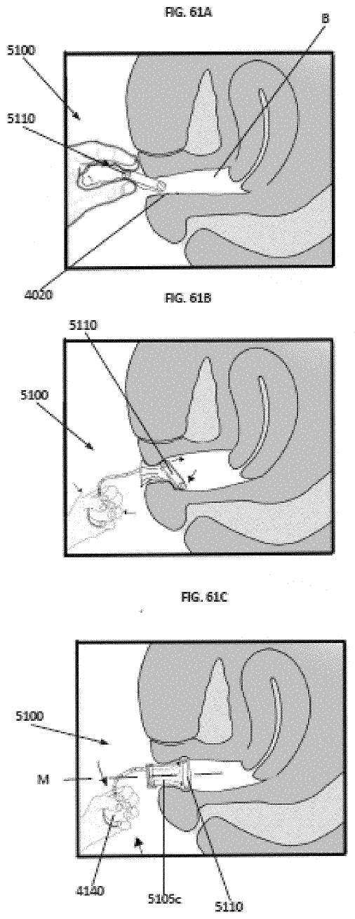

A medical device 5200′ according to another example is illustrated in FIGS. 63A-63C. Similar to medical device 5200, medical device 5200′ may include proximal ring 5205, an inflatable body 5210′, and lumens 5215 extending through inflatable body 5210′. Medical device 5200′ may also include a plurality of ribs 5220 extending radial outward from an outer surface of inflatable body 5210′. Alternatively, ribs 5220 may be formed integral with inflatable body 5210′, such that a shape of inflatable body 5210′ of medical device 5200′ includes ribs 5220 extending radially outward.

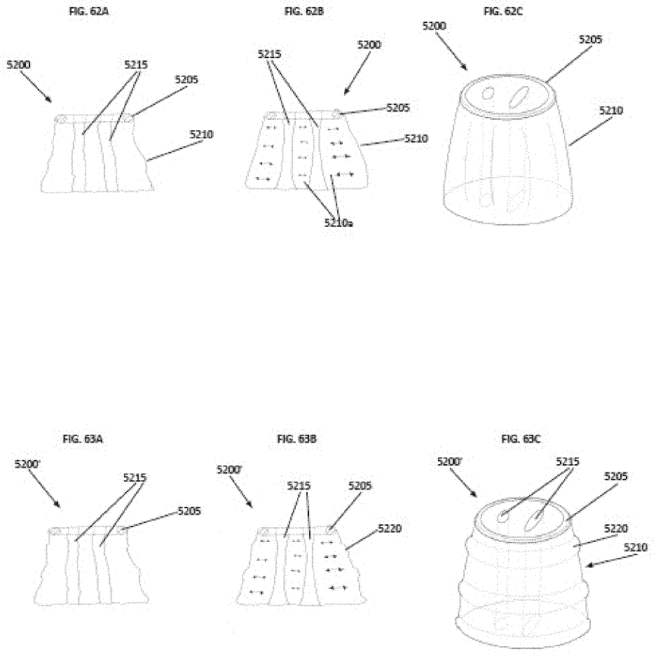

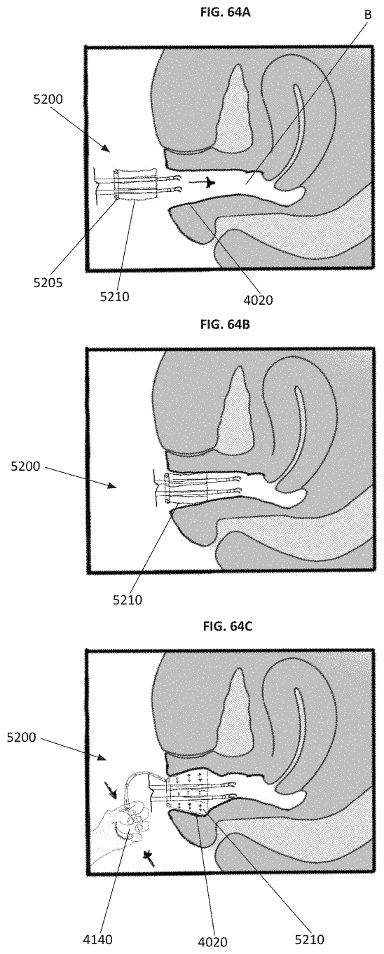

A medical procedure using either medical device 5200 or medical device 5200′ is shown in FIGS. 64A-64C (medical device 5200′ is not labeled in FIGS. 64A′64C, but a procedure using medical device 5200′ is similar as the procedure described relative to medical device 5200). Medical device 5200 may be inserted into body opening 4020. Medical tools (e.g., medical tools 4150 shown in FIG. 36C) may be inserted into lumens 5215 before or after medical device 5200 is inserted into body opening. Once inserted into body opening 4020, inflatable body 5210 may be inflated, which may assist in securing medical device 5200 in body opening 4020. The medical tools may then be used to perform a medical procedure. Inflatable body 5210 may be deflated before removing medical device 5200 from body opening 4020.

A medical device 5300 according to another example is shown in FIGS. 65A and 65B. Medical device 5300 may be similar to medical devices 5200 and 5200′. For example, medical device 5300 may include an inflatable body 5310 having a plurality of lumens 5315 extending from an opening in a proximal side to an opening in a distal side of inflatable body 5310. Inflatable body 5310 may be generally cylindrical shaped and may have a minimal height. For example, medical device 5300 may be positioned at a proximal end of a body opening (e.g., body opening 4020 in FIGS. 66A and 66B), as opposed to extending into a significant portion of the body opening. The reduced size of medical device 5300 may provide improved movement of medical tools (e.g., medical tools 4150 in FIG. 36C) extending through lumens 5315. As with other medical devices, pump 4140 may be used to inflate and/or deflate medical device 5300.

A method of performing a medical procedure using medical device 5300 will be described relative to FIGS. 66A and 66B. Medical device 5300 may be inserted into body opening 4020 in an uninflated configuration as shown in FIG. 66A. Inflatable body 5310 may be inflated with pump 4140. Medical tools 4150 may extend through lumens 5315 before or after medical device 5300 is inserted in body opening 4020 and inflated. Medical tools 4150 may perform a medical procedure. Upon completion of the medical procedure, inflatable body 5310 may be deflated and medical device 5300 may be removed from body opening 4020.

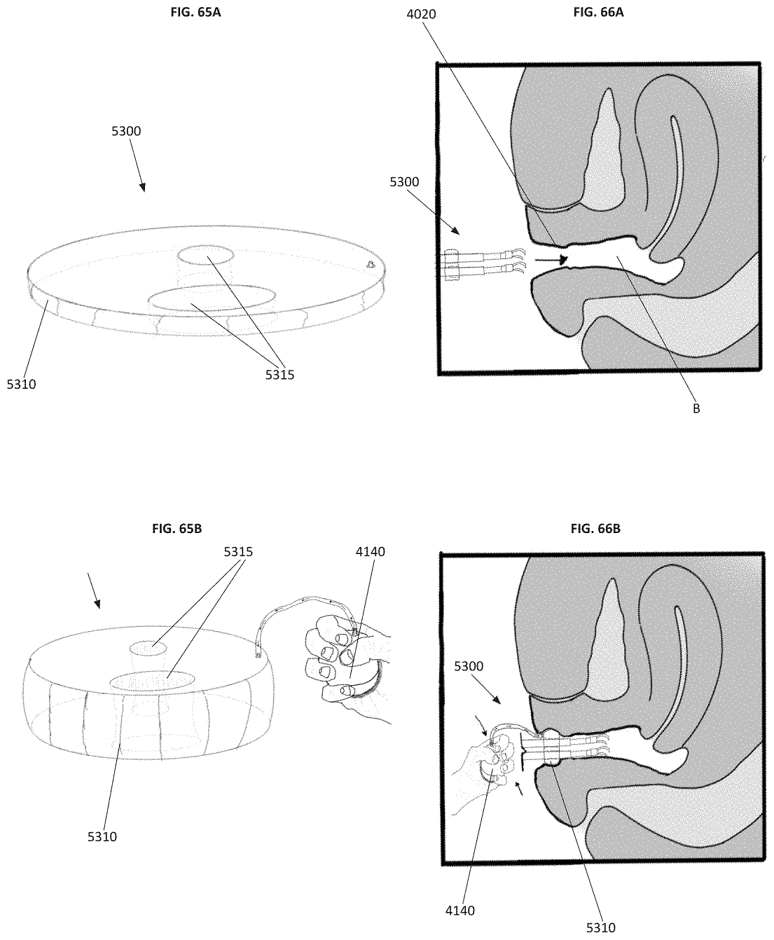

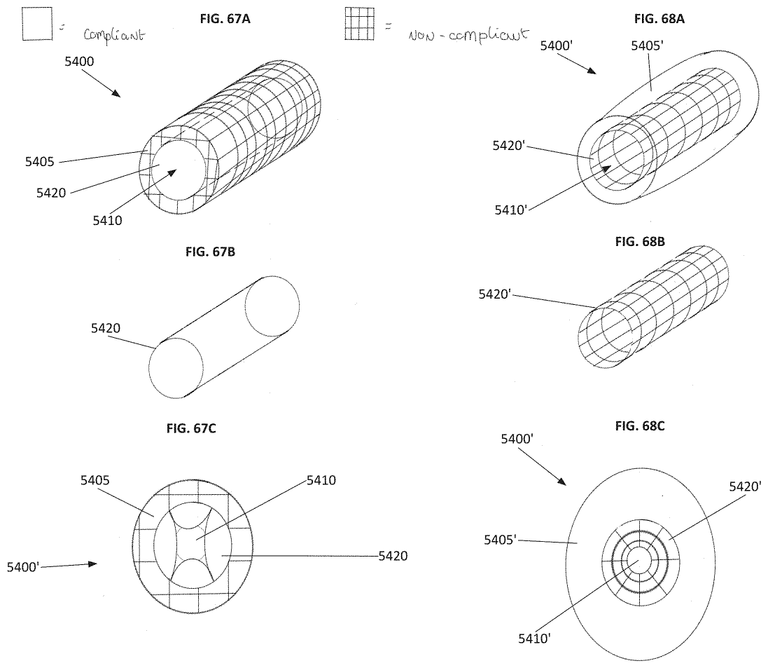

A medical device 5400 according to another example is shown in FIGS. 67A-67C. Medical device 5400 may be generally cylindrical in shape, but is not limited to that shape. Medical device 5400 may include an outer member 5405 including a non-compliant material. Outer member 5405 may define a lumen 5410. A radially outer surface of lumen 5410 may include an inner member 5420. Inner member 5420 may be formed of a compliant material. In some examples, compliant material may include a material that can inflate larger than its predetermined shape, similar to a typical party balloon. Some compliant materials may include inherent elasticity properties, such as elastic TPU, silicone, rubber, nitrile, late, or the like. Non-compliant materials may be those materials that inflate to a pre-determined shape and size determined by a construction of the device. Some non-compliant materials include non-elastic TPU, PVC, ripstop nylon, or the like. Additionally, or alternatively, the non-compliant and compliant sections may be combined, and/or may be switched (e.g., those sections that are shown as including compliant materials may include non-compliant materials, and vice-versa).

A size and shape of outer member 5405 may be preset and may allow outer member 5405 to be inflated to a specific size and shape. In contrast, the compliant material of inner member 5420 may continue to be inflated without a set size or shape. In some instances, a shape and a size of an inflated configuration of outer member 5405 may be preset based on, e.g., a size and a shape of the body opening. In this manner, outer member 5405 may be inflated when placed in the body opening. Inner member 5420 may also be inflated. Inner member 5420 may increase in size to surround one or more medical tools placed through lumen 5410. Medical device 5400 may be inserted in a body opening in a manner similar to those described herein.

A medical device 5400′ is illustrated with reference to FIGS. 68A-68C. Medical device 5400′ may include an outer member 5405′ including a compliant material, a lumen 5410′ defined by outer member 5405′, and an inner member 5420′ forming a surface of outer member 5405′ defining lumen 5410′. Inner member 5420′ may be formed of a non-compliant material. Similar to medical device 5400′, the size and shape of the non-compliant material may be preset. This may allow inner member 5420′ to be inflated to a preset size and shape to receive, e.g., a medical tool. Outer member 5405′ may be inflated without a preset size or shape based on the compliant material. This may be suitable in certain situations to allow medical device 5400′ to conform to a size and a shape of body opening.

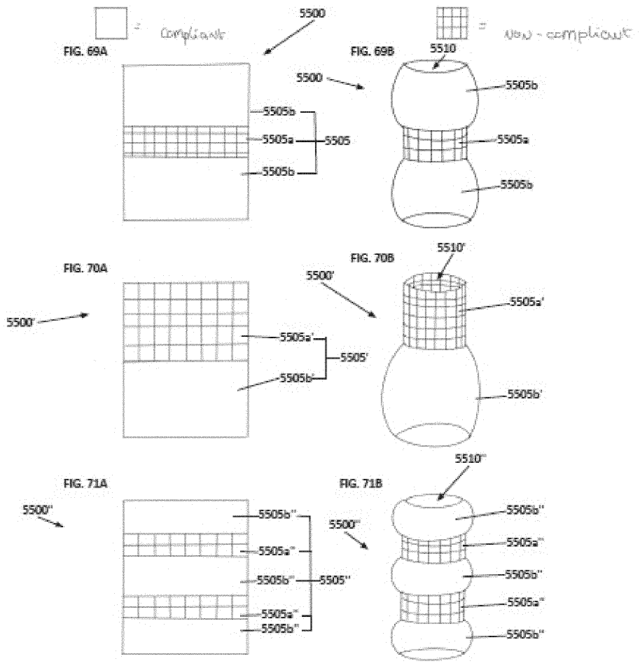

Three additional medical devices are shown in FIGS. 69A, 69B, 70A, 70B, 71A, and 71B. These medical devices may be similar to medical devices 5400 and 5400′ in that apportion of the devices may include a compliant material and a portion may include a non-compliant material. For example, medical device 5500 may be formed from a sheet 5505 shown in FIG. 69A. Sheet 5505 may include a non-compliant section 5505a sandwiched between two sections 5505b formed of compliant material. Two edges of sheet 5505 may be joined to form a lumen 5510 defined by sheet 5505. This configurations may allow medical device 5500 to be inflated such that distal and proximal ends are inflated more than a central portion of medical device 5500, as shown in FIG. 70B.

FIGS. 70A and 70B illustrate a medical device 5500′ having a similar design as medical device 5500. A sheet of material 5505′ may include non-compliant portion 5505a′ at a proximal end and a compliant portion 5505b′ at a distal end (or vice versa). Similar to medical device 5500, medical device 5500′ may include a lumen 5510′ when edges of sheet 5505′ are joined. This may cause medical device 5500′ to have a bottle shape as shown in FIG. 70B.

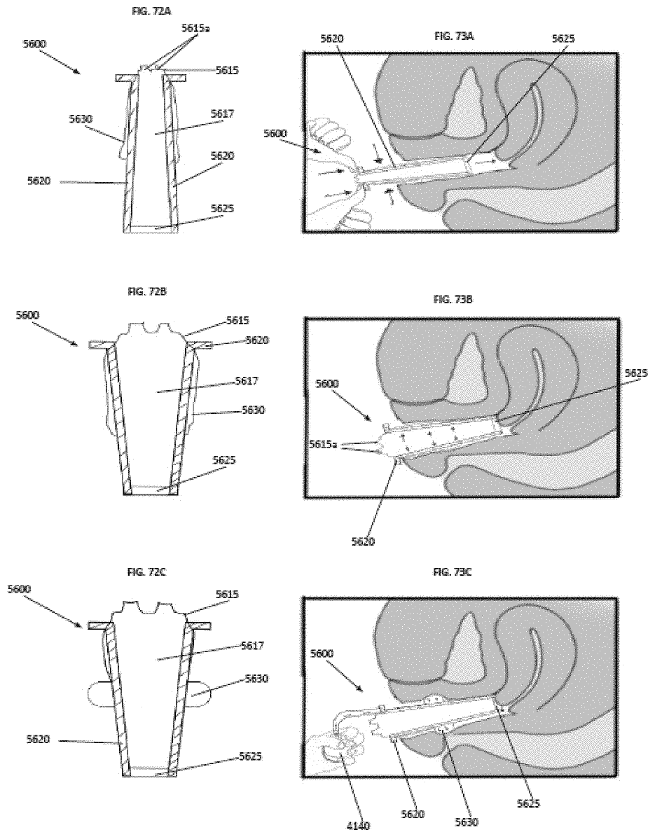

FIGS. 71A and 71B illustrate another example of a medical device 5500″. Medical device 5500″ may include a sheet of material 5505″ having alternating non-compliant portions 5505a″ and compliant portions 5505b″. While three compliant portions 5505b″ and two non-compliant portions 5505a″ are shown, medical device 5500″ is not limited thereto. Similar to medical devices 5500 and 5500′, medical device 5500″ may include a lumen 5510″ when edges of sheet 5505″ are joined. This may create outer rings of different diameters, which may assist in securing medical device 5500″ in a body opening. For example, the rings formed on an outer surface of medical device 5500″ may increase friction and/or may interact with tissue in the body opening to secure medical device 5500″ in the body opening.

It will be understood that medical tools may access a body cavity via the lumens extending through each of medical devices 5500, 5500′, and 5500″.

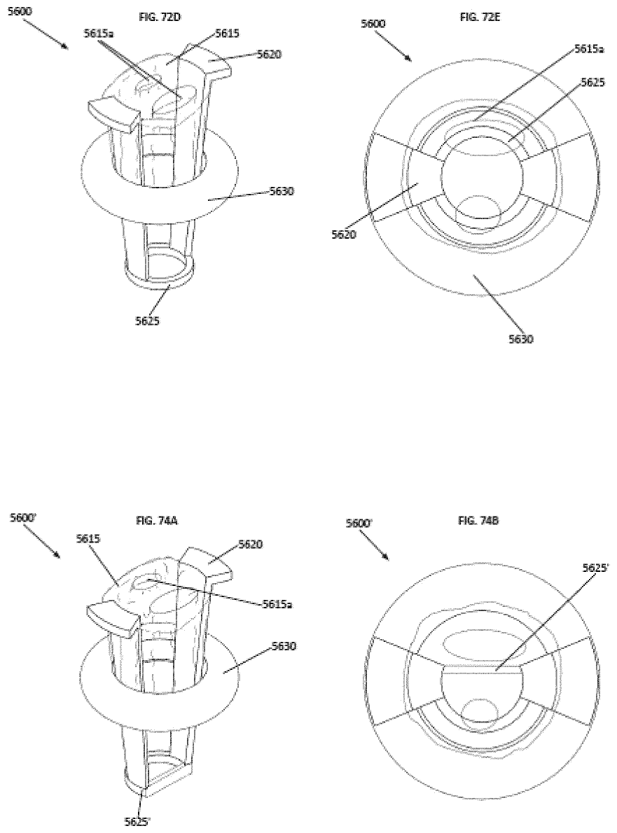

A medical device 5600 according to another example is shown in FIGS. 72A-72E. Medical device 5600 may include a pair of tabs or legs 5620. A flexible cap 5615 may be attached to each of tabs 5620 at a proximal end thereof. A lumen 5617 may extend between tabs 5620 from cap 5615 to a distal end of tabs 5620. Cap 5615 may seal an opening in the proximal end of tabs 5620. Cap 5615 may include a plurality of ports 5615a which may provide access to lumen 5617. A colpotomy member 5625 may be attached to the distal ends of tabs 5620. Tabs 5620 may be biased radially outward at one or more points along their length by a material and/or a design of tabs 5620. An inflatable member 5630 may be attached to an outer surface of each of tabs 5620 and may circumscribe lumen 5617, as shown in FIG. 72C. This configurations may assist in a colpotomy because it enables the user to remove a uterine manipulator without removing the entire port (e.g., it is unnecessary to remove colpotomy device with this design). Since a fluid seal may be maintained through the medical procedure, pneumo may be maintained. This may reduce a time associated with the procedure may be make the medical procedure safer by not needing to insert and remove additional medical instruments.

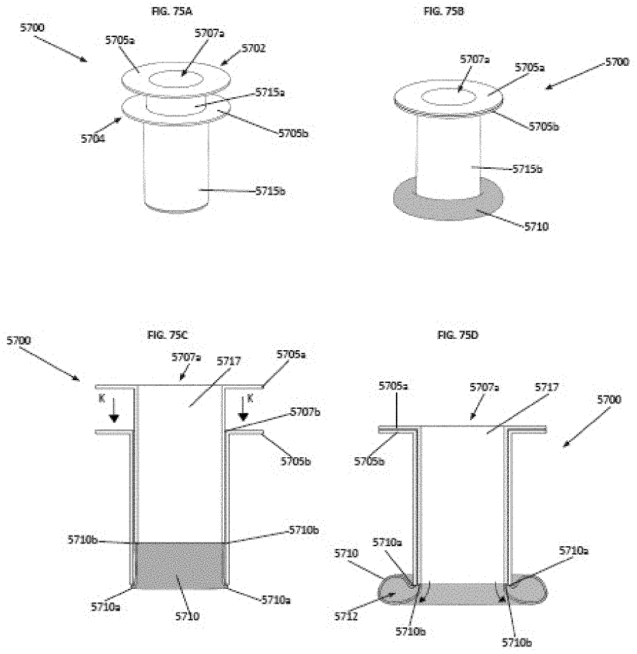

A method of performing a medical procedure using medical device 5600 is shown in FIGS. 73A-73C. Medical device 5600 may be inserted to body opening 4020, by inserting colpotomy member 5625 (e.g., a colpotomy cup). Medical device 5600 may be advanced distally until colpotomy member 5625 is appropriately positioned within body cavity B, as shown in FIG. 73B. In some instances, tabs 5620 may expand radially outward and may increase a cross-sectional area of a portion of lumen 5617. Subsequently, the user may inflate inflatable member 5630 using pump 4140 or the like. Tools may be inserted into body cavity B via ports 5615a and lumen 5617. To remove medical device 5600, inflatable member 5630 may be deflated and medical device 5600 may be moved in a proximal direction out of body opening 4020.

According to another example, a medical device 5600′ is shown in FIGS. 74A and 74B. Medical device 5600′ may be similar to medical device 5600 described with reference to FIGS. 72A-72E and may have similar features. Notably, colpotomy member 5625′ in medical device 5600′ may be semi-circular in cross section, as opposed to the annular ring of colpotomy member 5625 of medical device 5600. This may allow medical tools or instruments to have greater freedom of movement in ports 5615a and lumen 5617 during a medical procedure. A method of using medical device 5600′ may be similar to the medical procedure described for medical device 5600 with reference to FIGS. 73A-73C.

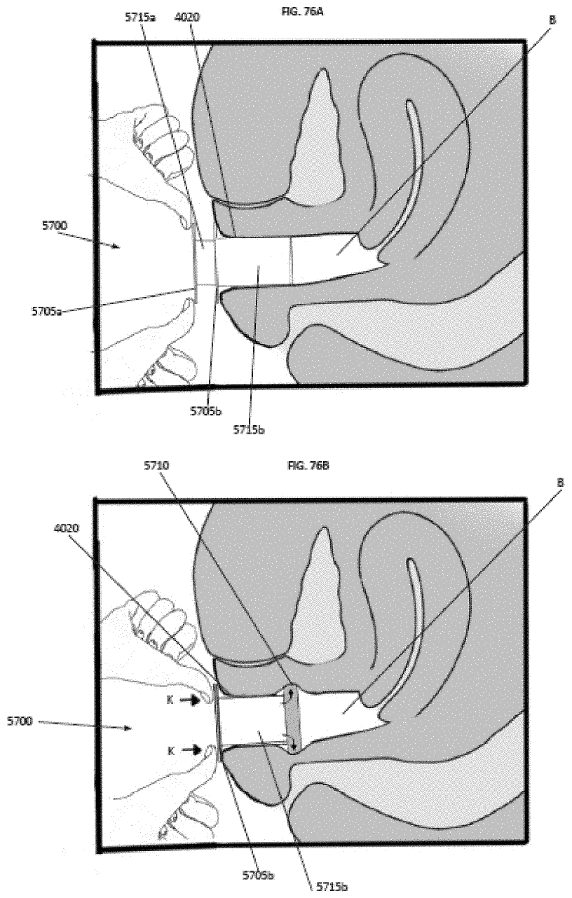

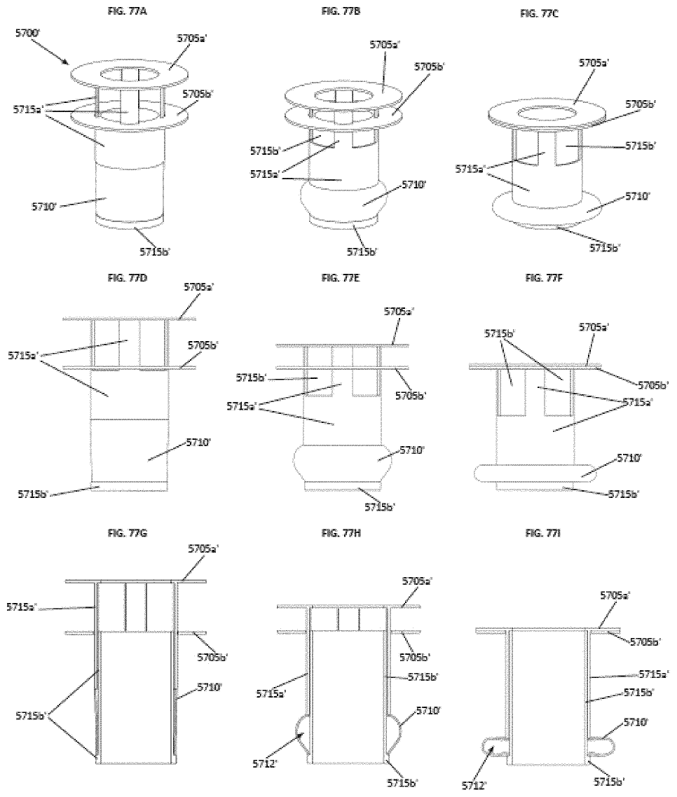

A medical device 5700 according to another example is shown in FIGS. 75A-75D. Medical device 5700 may include a first member 5702 having a proximal ring or flange 5705a and an annular member or cylindrical tube 5715a extending distally from a distal end of proximal ring 5705a. Proximal ring 5705a may define an opening 5707a connected to a first lumen 5717 defined by annular member 5715a. Medical device 5700 may also include a second member 5704 having a proximal ring or flange 5705b and an annular member or cylindrical tube 5715b extending distally from a distal end of proximal ring 5705b. Proximal ring 5705b may also define an opening 5707b connected to a second lumen defined by annular member 5715b. A diameter of an outer wall of annular member 5715a may be smaller than a diameter of an inner wall of annular member 5715b. This may allow first member 5702 to be positioned within the second lumen and be coaxial with second member 5704.

Medical device 5700 may also include an expandable member 5710. A first end 5710a of expandable member 5710 may be attached to a distal end of annular member 5715b. A second end 5710b of expandable member 5710 may be attached to a distal end of annular member 5715a. Movement of annular member 5715b relative to annular member 5715a, as shown by arrows K in FIG. 75C, may cause expandable member 5710 to move from a position generally aligned along a central axis of lumen 5717 (FIG. 75C) to an expanded position (FIG. 75D). Expandable member 5710 may form a ring-like member in the expanded position, and may create a pocket 5712. The expanded shape may take different forms, and may be a pre-formed shape, such as a pre-formed ring shape that is biased to extend radially outwardly as shown in FIGS. 75B and 75D. Pocket 5712 may include a fluid (e.g., air, water, etc.) which may assist in expanding the expandable member 5710. Rather than expanding with a fluid, the expandable member 5710 may be formed by a rigidity of the material, e.g., rubber, silicone of sufficient rigidity to allow expansion via movement of annular members 5715a and 5715b, and to form a securing element within a body opening. In this arrangement, expandable member 5710 may be a pre-formed shape, such as a pre-formed ring shape that is biased to extend radially outwardly as shown in FIGS. 75B. In this manner, expandable member 5710 may assist in securing medical device 5700 in a body opening.

A medical procedure using medical device 5700 is described with reference to FIGS. 76A and 76B. Medical device 5700 may be inserted into body opening 4020 by pushing on a proximal end of medical device 5700, as shown in FIG. 76A. Once seated in body opening 4020, a larger force may be supplied to the proximal end of medical device 5700 to, e.g., proximal ring 5705a. This may cause proximal ring 5705a to move toward proximal ring 5705b, as indicated by arrows K in FIG. 76B. FIG. 76A may indicate proximal ring 5705a prior to moving towards proximal ring 5705b. This relative movement may cause 5710 to move to the expanded configurations and may secure medical device 5700 within opening 4020. A ratchet-type or other appropriate securement system (not shown) may be included on annular members 5715a and 5715b to maintain the annular members 5715a and 5715b in the expanded configurations (FIG. 75D). Medical tools may be inserted into the lumen of medical device 5700. After the medical procedure is complete, proximal ring 5705a may be moved proximally, away from proximal ring 5705b. This may move expandable member 5710 to the unexpanded state, thereby allowing a user to remove medical device 5700 from body opening 4020.

A medical device 5700′ according to another example is shown in FIGS. 77A-77I. Medical device 5700′ may be similar to medical device 5700 described in FIGS. 75A-75D. Medical device 5700′ may include a proximal ring or flange 5705a′ having an annular member or cylindrical tube 5715a′ extending from a distal end of proximal ring 5705a′. A portion of annular member 5715a′ may be formed as struts, which may extend through and may move within openings in a proximal ring or flange 5705b′. A distal end of annular member 5715a′ may be connected to a proximal end of an expandable member 5710′.

An annular ring or cylindrical tube 5715b′ may extend from a distal end of proximal ring 5705b′. A diameter of an outer surface of annular member 5715b′ may be different along its length. For example, a diameter of an outer surface at a distal end of annular member 5715b′ may have a diameter greater than a diameter of an outer surface of annular member 5715b′ at a proximal location. The distal end of annular member 5715b′ may be attached to a distal end of expandable member 5710′. Annular member 5715a′ may be coaxial with annular member 5715b′ and may slide over a portion of an outer surface of annular member 5715b′, as described below.

FIG. 77A illustrates a configuration in which an outer surface of each of the distal end of annular member 5715b′, expandable member 5710′, and annular member 5715a′ are aligned. As proximal ring 5705a′ is moved toward proximal ring 5705b′, as shown in FIG. 77B, expandable member 5710′ may expand or bulge radially outward to an expanded state, as shown in FIG. 77C. As medical device 5700′ is transitioned from the unexpanded configurations to the expanded configurations, an outer surface of annular member 5715b′ may be viewed between struts of annular member 5715a′. In some instances, struts of annular member 5715a′ may interact with the openings in proximal ring 5705b′ in a ratchet-type configurations. This may enable expandable member 5710′ of medical device 5700′ to be expanded at different positions, which may assist in securing medical device 5700′ in a body opening. As described with respect to medical device 5700, expandable member 5710′ may include a pocket 5712′ which may include a fluid to assist in expanding expandable member 5710′. FIGS. 77D-77I illustrate additional view of medical device 5700′ in the expanded and an unexpanded configurations.

A medical procedure using medical device 5700′ may be performed in a manner similar to the medical procedure using medical device 5700. For example, medical device 5700′ may be inserted into a body opening by pushing on a proximal end of medical device 5700′. Once seated in the body opening, a larger force may be supplied to the proximal end of medical device 5700′ to, e.g., proximal ring 5705a′. This may cause proximal ring 5705a′ to move toward proximal ring 5705b′. The position of proximal ring 5705a′ and 5705b′ may be changed based on the ratchet-like mechanism. This may enable a user to expand expandable member 5710′ at different rates and use medical device 5700′ in different sized body openings. Medical tools may be inserted into the lumen of medical device 5700′. After the medical procedure is complete, proximal ring 5705a′ may be moved proximally, away from proximal ring 5705b′. This may move expandable member 5710′ to the unexpanded state, thereby allowing a user to remove medical device 5700′ from the body opening.

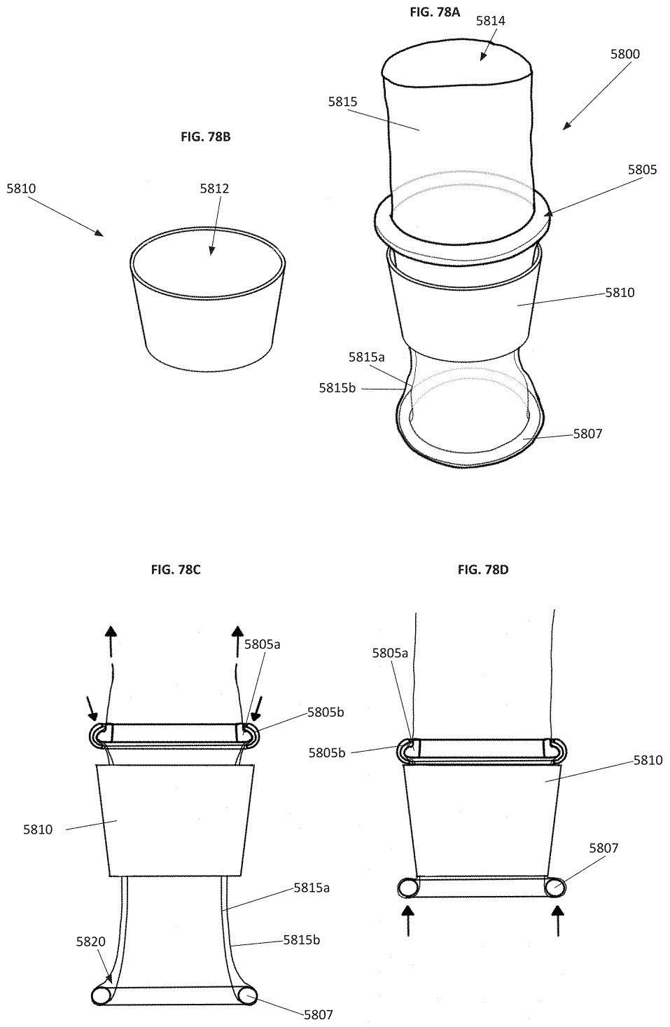

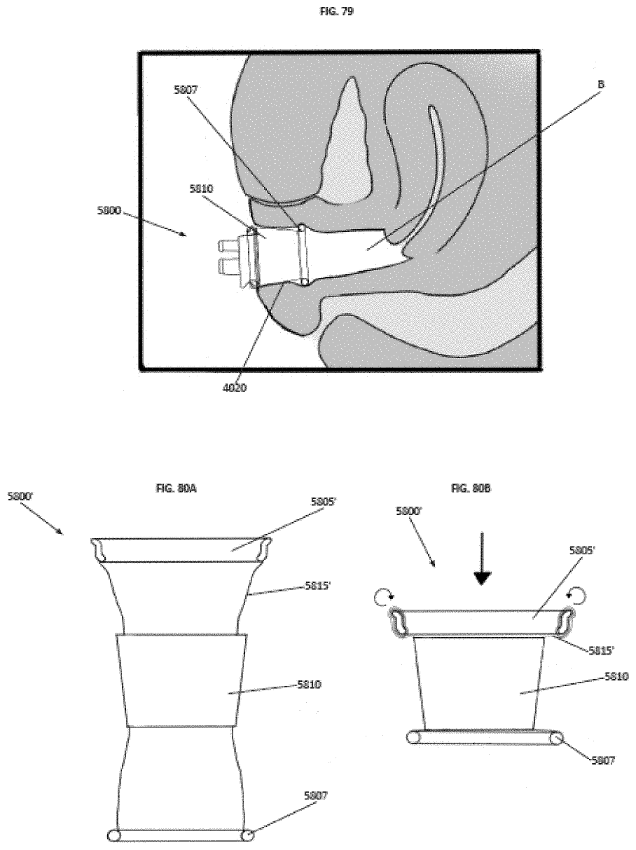

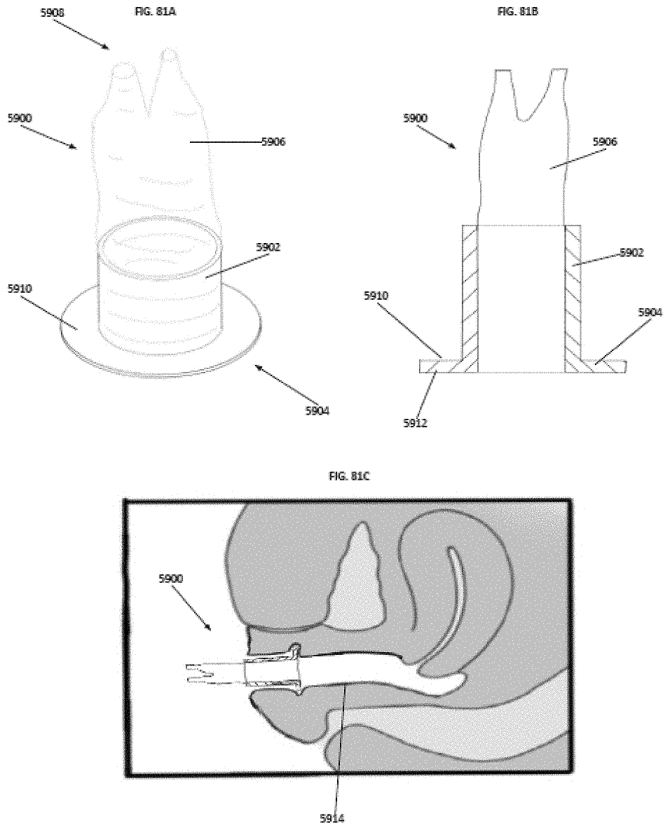

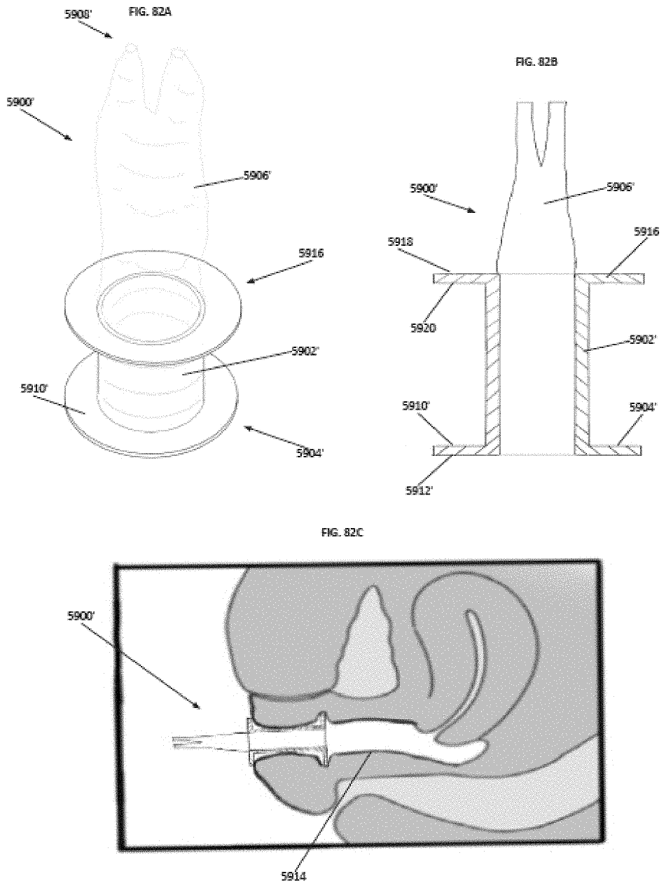

A medical device 5800 according to another example is shown in FIGS. 78A-78D. Medical device 5800 may include a retractor having a sleeve 5815 connected at a first end to an outer proximal ring 5805b. Sleeve 5815 may loop around a distal ring 5807, through an opening in distal ring 5807, and back on itself. In other words, sleeve 5815 may include a first radially outer layer 5815b and a second radially inner layer 5815a. Sleeve 5815 may extend back through an opening in proximal ring 5805. A lumen 5820 may be formed through sleeve 5815 to allow for access to a target site when medical device 5800 is placed in a body, as described below. An outer member 5810, shown in FIG. 78B, may include a lumen 5812 and may be coaxial with sleeve 5815. Outer member 5810 may be cone shaped, a truncated cone, or a frustoconical shape, but is not limited thereto (e.g., cylindrical, cuboidal, etc.) Outer member 5810 may be rigid or semi-rigid and may provide a fixed-length of medical device 5800. As shown in FIG. 78C, sleeve 5815 may extend between outer proximal ring 5805 and an inner proximal ring 5805a. Sleeve 5815 may be moved in a proximal direction to shorten a distance between proximal rings 5805a, 5805b and distal ring 5807. Outer member 5810 may determine a maximum distance distal ring 5807 may approach proximal rings 5805a, 5805b, as shown in FIG. 78D.

A medical procedure using medical device 5800 is shown with reference to FIGS. 79. Medical device 5800 maybe inserted into body opening 4020 to access cavity B. Medical device 5800 may be inserted in an expanded configurations (FIG. 78D). Movement of outer member 5810 in a proximal direction may cause distal ring 5807 and proximal rings 5805a, 5805b to move closer together until each of distal ring 5807 and proximal rings 5805a, 5805b may abut an end of outer member 5810. A cap may be attached to a proximal end of medical device 5800 which may seal a proximal end of lumen 5814. This may prevent pneumo from escaping body cavity B during the medical procedure. After the medical procedure is complete, distal ring 5817 may be pulled in a proximal direction to remove medical device 5800 from body opening 4020.

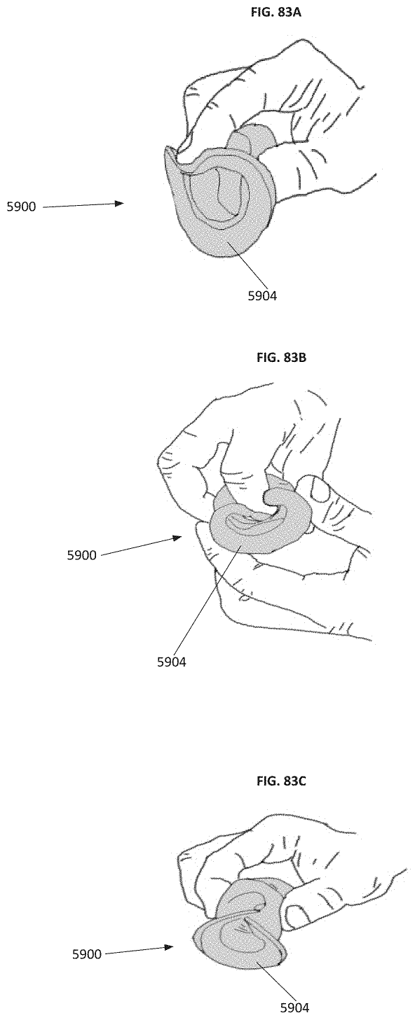

A medical device 5800′ according to another example is shown in FIGS. 80A and 80B. A sleeve 5815′ is attached at a proximal end to a proximal ring 5805′ and at a distal end to a distal ring 5807. Outer member 5810 may be coaxial with sleeve 5815′. Sleeve 5815′ may extend through an opening in outer member 5810. Distal ring 5807 may be pulled closer to proximal ring 5805′ by rolling proximal ring 5805′. Proximal ring 5805′ may be rolled until proximal ring 5805′ and distal ring 5807 abut opposite ends of outer member 5810. A medical procedure us medical device 5800′ may be performed in a manner similar to the manner described for using medical device 5800 with reference to FIG. 79.

A medical device 5900 according to another example is shown in FIGS. 81A-81C. In a first arrangement, medical device 5900 includes a semi-rigid cylindrical or conical member 5902 with a flange 5904 at its distal end. A sleeve 5906 can be secured at a proximal end of the medical device 5900, and the sleeve may include a valve system (not shown) at a proximal end 5908 for the insertion of instrumentation. The flange 5904 may extend transversely from the cylindrical member 5902 and may include opposite planar surfaces 5910, 5912, as shown. Flange 5904 may form a step up in diameter from that of the cylindrical member 5902. Cylindrical member 5902 and flange 5904 may be integrally formed, or separate members fixedly connected together, and each may be formed of a semi-rigid material, such as TPU, silicone, rubber, nitrile, or the like.

As shown in FIG. 81C, flange 5904 may help to anchor medical device 5900 within the vaginal canal 5914. Such anchoring facilitates the maintaining of a pneumoperitoneum in the vaginal canal 5914 by helping to prevent rotating or tilting of the medical device 5900 that may allow insufflation gas to escape around the outside of the medical device 5900. Further, the shape of the cylindrical member 5902 naturally aligns itself to the vaginal canal 5914, and therefore further helps prevent the distal flange 5904 from rotating/tilting within the canal 5914. FIG. 81B shows a sectional view of medical device 5900. FIG. 81C shows a sectional view of the medical device 5900 within the vaginal canal 5914.

A medical device 5900′ according to another example is shown in FIGS. 82A-82C. Medical device 5900′ is an iteration of medical device 5900, but with the addition of a second, proximal flange 5916 which sits against the vulva (FIG. 82C). Thus, the features and discussion above with respect to medical device 5900 are equally applicable to medical device 5900′ and the same reference numbers are used to indicate same components. The second flange 5916 may be located at a proximal end of cylindrical member 5902′ and may be similarly configured as distal flange 5904′. For example, proximal flange 5916 may extend transversely from the cylindrical member 5902′ and may include opposite planar surfaces 5918, 5920 as shown in FIG. 82B. Flange 5904 may form a step up in diameter from that of the cylindrical member 5902′. Cylindrical member 5902′ and flange 5916 may be integrally formed, or separate members fixedly connected together, and each may be formed of a semi-rigid material, such as TPU, silicone, rubber, nitrile, or the like.

Proximal flange 5916 may add extra stability against the tilting or rotation of the distal flange 5904′. FIG. 82B shows a sectional view of medical device 5900′. FIG. 82C shows a sectional view of the medical device 5900′ within the vaginal canal 5914.

FIGS. 83A-83C describe how to fold the semi rigid medical devices 5900 and 5900′ for ease of insertion into the vagina. Holding the cylindrical member 5902, 5902′ of the medical device, a user can use their index finger to push down and pull the flange 5904, 5904′ toward and into the cylindrical member 5902, 5902′. Continuing this motion allows folding of more than half of the flange 5904, 5904′ into the cylindrical member 5902, 5902′. Once fully folded, the folded flange 5904, 5904′ will have a reduced footprint by more than 50% and may facilitate insertion into the body opening (e.g., vagina), thereby minimizing trauma upon insertion into the vaginal canal 5914. Once within the vaginal canal 5914, the flange 5904, 5904′ can automatically restore (or be re-configured or urged) back to its original shape. These steps can similarly be used to remove the port from within the vaginal canal 5914.

The devices described herein may facilitate securely anchoring the devices into a body openings such as the vagina where there is not a tissue ledge or step on which to secure the device. This may be achieved, for example, by forming both: (a) a radial extendable or movable element that can allow for insertion and be extended or moved sufficiently in a radial direction to urge against the body opening, such as the vaginal canal, and (b) a stabilizing feature to increase the rigidity of the device and assist in preventing a tilting of the device that would allow gas to escape from around the device. For example, with respect to the device shown in FIGS. 35A-35D, a radially expandable element in the form of membrane 4115 can be extended sufficiently in a radially direction (e.g., by inflation), and tab or leg 4120 may form a stabilizing element to assist in keeping membrane (including distal ring 4110) from tilting in the body opening 4020. Medical device 4200 of FIGS. 37A-39C may include a pair of stabilizing elements via two or more tabs or legs 4220. Stabilizing feature of medical device 4300 may include tabs or legs 4320 and cap 4130. Similarly, other devices disclosed herein include stabilizing features or elements, such as tabs or legs 4420, 4520, 4620 and associated caps 4130; members 4707 and crossbars 4705 of the device of FIGS. 52A-52D; annular members 4805, 4905; chambers/inflatable body 5107, 5210, 5310; non-compliant sections 5505a; tabs 5620; outer member 5810; and semi-rigid cylindrical members 5902 (and proximal flange 5916).

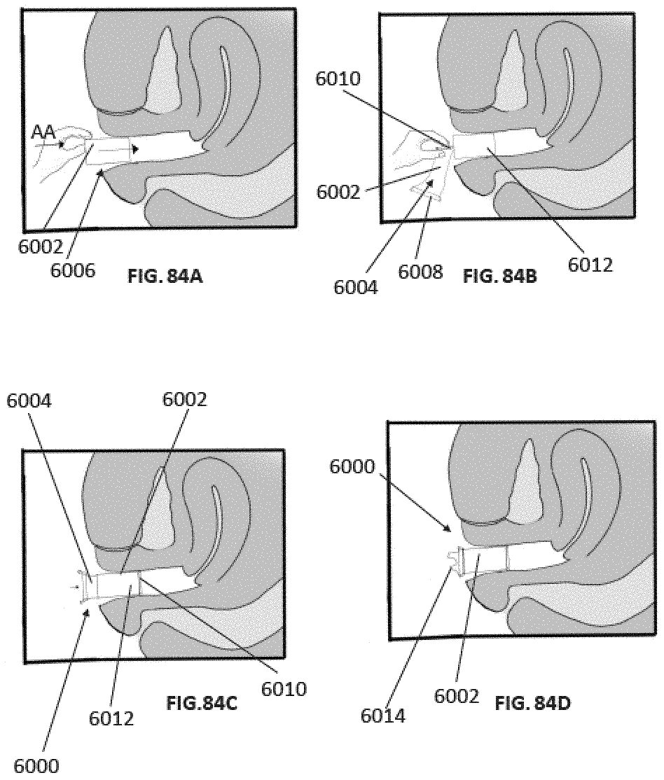

FIGS. 84A-84D illustrate a method of using a medical device 6000 in a body opening of a patient (e.g., transvaginal access). Medical device 6000 may be utilized for allowing sealed (or unsealed) insertion of surgical instruments, tools, or other equipment into the body opening 6006. A user may first insert an outer access member 6002 into a body opening 6006, e.g., a vagina, as indicated by arrows AA in FIG. 84A. The term outer member may also be referred to as conduit. The outer member 6002 may be a tube or conduit constructed of various shapes and sizes, some of which are discussed herein. The outer member 6002 may be rigid or semi rigid. A sleeve 6004, shown in FIG. 84B, may be separate from outer member 6002, and may form a part of medical device 6000. Sleeve 6004 may be an embodiment of sleeve 105. Sleeve 6004 may include a proximal ring 6008, e.g., a flexible O-ring at a proximal end of the sleeve 6004, and a distal ring 6010, e.g., flexible O-ring at a distal end of the sleeve 6004. The sleeve 6004 may take the form of a retractor device. In use, outer member 6002 may be located radially outside and coaxial with sleeve 6004. Medical device 6000 may refer to only the outer member 6002, or the combination of one or more of the outer member 6002, sleeve 6004, proximal ring 6008, distal ring 6010, and further additions, as will be described below.

FIG. 84B depicts a user inserting the distal ring 6010 and sleeve 6004 into the outer member 6002 until the distal ring 6010 exits out of a distal end 6012 of the outer member 6002. FIG. 84C illustrates how the distal ring 6010 will catch onto the distal end 6012 of the outer member 6002 when the sleeve 6004 is retracted, for example, by rolling the proximal ring 6008 towards the body opening 6006. Proximal ring 6008 may be rolled until proximal ring 6008 and distal ring 6010 abut the ends of the outer member 6002. The semi rigid or rigid structure of the outer member 6002, combined with the tension created by the rolling of the sleeve 6004, may ensure that the distal ring 6010 aligns perpendicularly to the body opening (e.g., vaginal wall) in a position conducive to sealing and anchoring. FIG. 84D illustrates the medical device 6000 in its final position with a valved sleeve end or separable valved cap 6014 coupled to proximal ring 6008. The valved sleeve end or cap 6014 may provide for sealed introduction of medical instruments, tools or other equipment (not shown) through the medical device 6000 to perform a medical procedure on a patient. Such sealed access may assist in maintaining fluid (e.g. insufflation gas) within the patient body during the medical procedure. Including a valved sleeve or attaching a valved cap 6014 to the medical device 6000 may be optional. After the medical procedure is complete, distal ring 6010 may be pulled in a proximal direction to remove medical device 6000 from body opening 6006. FIGS. 84A-84D depict a process that may be applied to describe all medical access devices described below. The process described in FIGS. 84A-84D may be applied before a medical device is inserted into a medical access device.

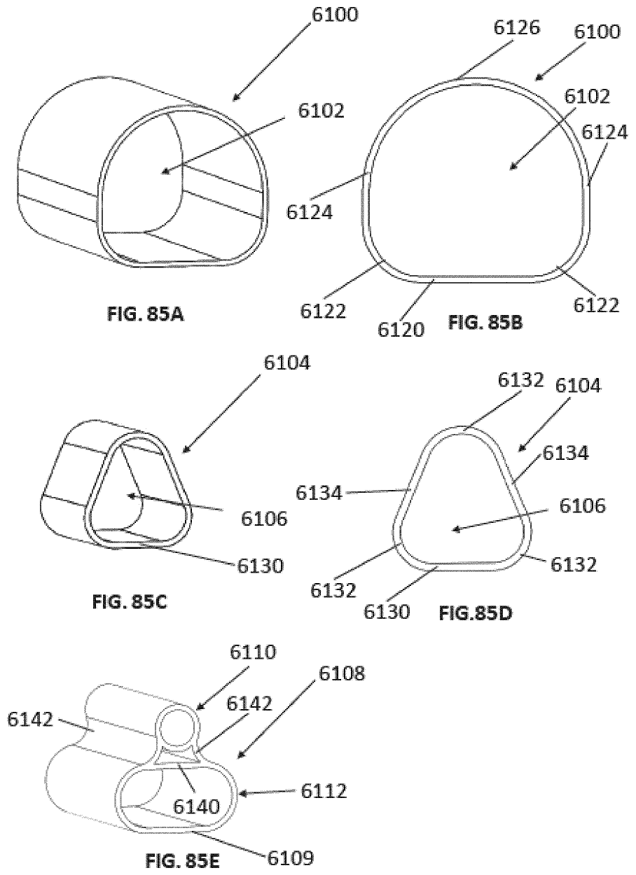

FIGS. 85A-85E depict various embodiments of outer access member 6002 shown in FIGS. 84A-84D. Outer member 6002 is not limited to the designs of FIG. 85A-85E. These embodiments may be semi rigid or rigid and may be simple tubular structures of varying sizes meant to fit a desired body opening, such as for transvaginal access. FIG. 85A depicts a first embodiment of an outer member 6100 which has opening 6102. FIG. 85B depicts an end view of outer member 6100. As shown, outer member 6100 may include a flat or straight bottom portion 6120, rounded corners 6122 from the base transitioning to straight or flat sides 6124, and a rounded top 6126 connecting the sides 6124. The rounded top 6126 of the outer member 6100 may have a constant radius. Also, flat sides 6124 may be shorter, the same size, or larger than the bottom portion 6120. Further, outer member 6100 may have a constant cross-section from the proximal to distal ends.

FIG. 85C depicts a second, triangular, embodiment of an outer member 6104 which has an opening 6106. FIG. 85D depicts an end view of outer member 6104. As shown, triangular outer member 6104 may include a bottom portion 6130 curved or rounded corners 6132, and straight sides 6134. Flat sides 6134 may be shorter, the same size, or larger than the bottom portion 6130. Further, outer member 6104 may have a constant cross-section from the proximal to distal ends.

FIG. 85E depicts a generally triangular embodiment of an outer member 6108 of a medical access device. Outer member 6108 contains an upper tubular member 6110 connected to a lower stadium-shaped member 6112. The tubular member 6110 may be centrally connected to one of the flat sides 6134 of the stadium-shaped member 6112 by a pair of connecting strips 6142. The pair of connecting strips 6142 may have a concave shape. The stadium-shaped member 6112, and upper tubular member 6110 may form separate openings for insertion of the medical instruments or tools. As shown, the cross-section size of the tubular member 6110 may be smaller than the cross-sectional size of the stadium-shaped member 6112.

In outer member 6104 and outer member 6108, the generally triangular profile may ensure a strong wide opening on the bottom portions 6130 and 6109. The stronger bottom portions 6130 and 6109 may facilitate the use of certain instrumentation or surgical operating practices that require more space in the anterior or posterior sides of the vaginal cavity.

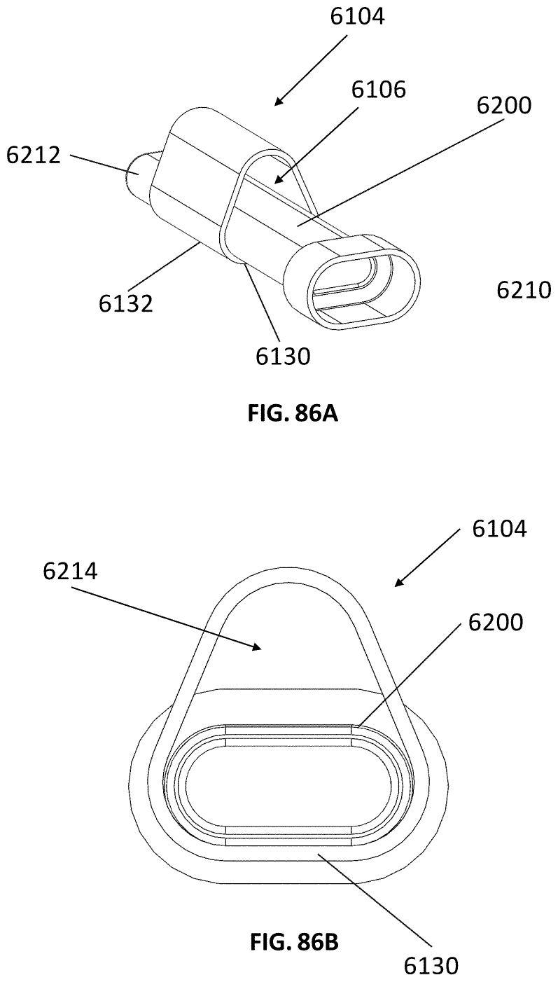

FIGS. 86A-86B illustrate medical access device 6104 of FIGS. 85C and 85D in use with an oval shaped medical instrument or tool 6200. FIG. 86A shows a perspective view of the combination medical access device 6104 and medical instrument 6200. The oval shaped medical instrument 6200 may take the form of a cannula having a proximal enlarged oval end 6210, and an oval tube 6212 extending distally from proximal end 6210. The medical instrument 6200 may be inserted into the outer member opening 6106. When inserted, medical instrument 6200 may abut the bottom portion 6130 and the curved or rounded corners 6132. The medical instrument 6200 may be kept in position posteriorly (or anteriorly if otherwise orientated) due to the narrow nature of the triangle structure. When the outer member 6104 and medical instrument 6200 are in-situ in a vagina or in an alternative orifice or opening, a lumen 6214 may be formed above medical instrument 6200. This lumen 6214 may be a maximized space above the medical instrument 6200 due to the interaction of the shape of the medical instrument 6200 and the outer member 6300.

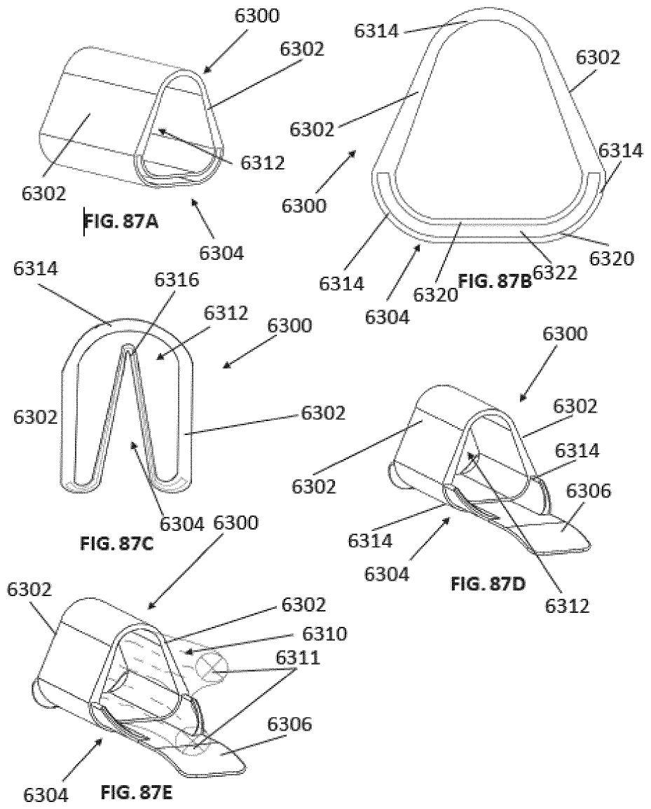

FIGS. 87A-87E illustrate alternative components of a medical access device for use with a body opening. In particular, FIGS. 87A-87E depict alternative embodiments of outer member 6002 and 6104 from FIGS. 84A-84D and 85C-85D, and is referred to as outer member 6300. As shown in FIG. 87B, triangular outer member 6300 may include a folding base 6304, curved or rounded corners 6314, and straight sides 6302 which create an opening 6312. The straight sides 6302 may be shorter, the same size, or larger than folding base 6304. Further, outer member 6300 may have a constant cross-section from the proximal to distal ends when not bent. The folding base 6304 may be constructed of two thin walls 6320 and an opening 6322 located between the two thin walls 6320. The thin walls 6320 may wrap around the rounded corner 6314 attached to the folding base 6304. The folding base 6304 may be more flexible compared to the straight sides 6302. This may allow folding base 6304 to fold in and bend at a folding point 6316 as shown in FIG. 87C. When folding base 6304 is bent, folding point 6316 may be moved closer to the rounded corner 6314 located between the straight sides 6302. This folded outer member 6300 may be more optimally sized for insertion of outer member 6300 into the vagina as shown in FIG. 87C. After outer member 6300 has been inserted into the vagina, a rigid shoe horn like stabilizing component 6306 can be fed through the opening 6312 to reestablish a strong wall at the folding base 6304 as shown in FIG. 87D. The component 6306 may extend outside the outer member 6300 on both sides of the outer member 6300. FIG. 87E shows an embodiment of the outer member 6300 with valved sleeved 6310 attached. The valved sleeve 6310 may have one or more opening 6311 which may allow for sealed insertion of medical instruments.

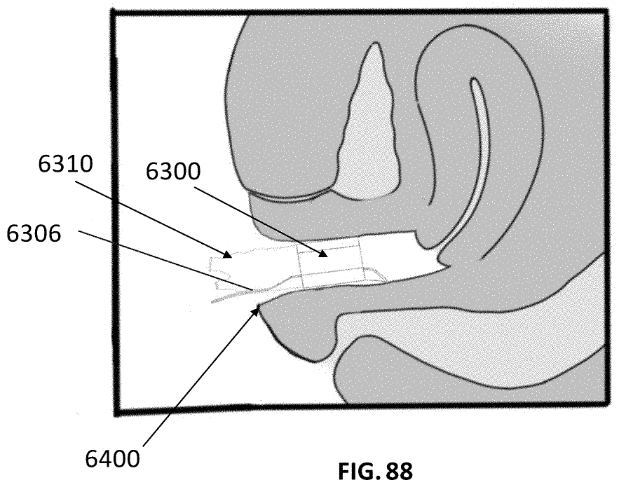

FIG. 88 depicts the outer member 6300 and stabilizing component 6306 of FIGS. 87D or FIG. 87E located in-situ within a body opening 6400 (e.g. a vagina).

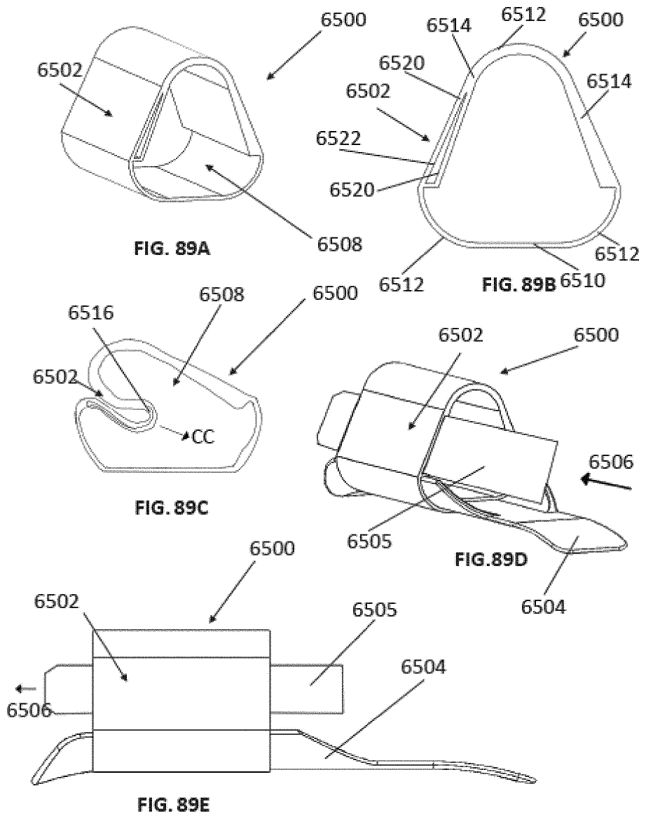

FIGS. 89A-89E illustrate alternative components of a medical access device for use with a body opening. In particular, FIGS. 89A-89E may depict alternative embodiments of outer member 6002 and 6104 shown in FIGS. 84A-84D and 85C-85D and referred to here as outer member 6500. Similar to outer member 6104, 6500 is made up of a straight base 6510, curved or rounded corners 6512, and a pair of straight sides 6514 which form an opening 6508. Outer member 6500 is differentiated from outer member 6300 from FIGS. 87A-87E, in that the folding sidewall 6502 is not located at the straight base 6510, but instead is located on one of the straight sides 6514. Folding sidewall 6502 may be constructed of two thinner walls 6520 which contain an opening 6522 located between thin walls 6520. The opening 6522 may be widest closest to the straight base 6510 and the width of opening 6522 may narrow the farther the opening 6522 is from the straight base 6510. FIG. 89C shows the outer member 6500 with the folding sidewall 6502 in a folded position. A folding point 6516, located along folding sidewall 6502 may be bent and brought towards the rounded corner 6512 opposite of the folding sidewall 6502 as demonstrated by arrows CC. The folded position may distort the opening 6508 and may allow for easier insertion of outer member 6500 into a body opening such as a vagina. FIGS. 89D-89E depict the outer member 6500 with a rigid shoe horn like component 6504 located within the outer member 6500. The component 6504 may conform to abut the straight base 6510 and bottom corners 6314. The component 6504 may extend outside the outer member 6500 on both sides of the outer member 6500. The component 6504 may help provide support for the outer member 6500. Additionally, a second rigid planar member (e.g., tongue depressor-like stick) 6505 may be inserted into outer member 6500 and be attached to the folding sidewall 6502 to help reestablish the structure of 6500 as shown in FIGS. 89D-89E. The planar member 6505 may for a stiffening member for outer member 6500 to help strengthen folding sidewall 6502. The planar member 6505 may be generally rectangular in shape as shown. Arrows 6506 may show the direction in which planar member 6505 is inserted. The component 6504 and planar member 6505 may be inserted into outer member 6500 after outer member 6500 has been inserted within an opening in a body (not shown).

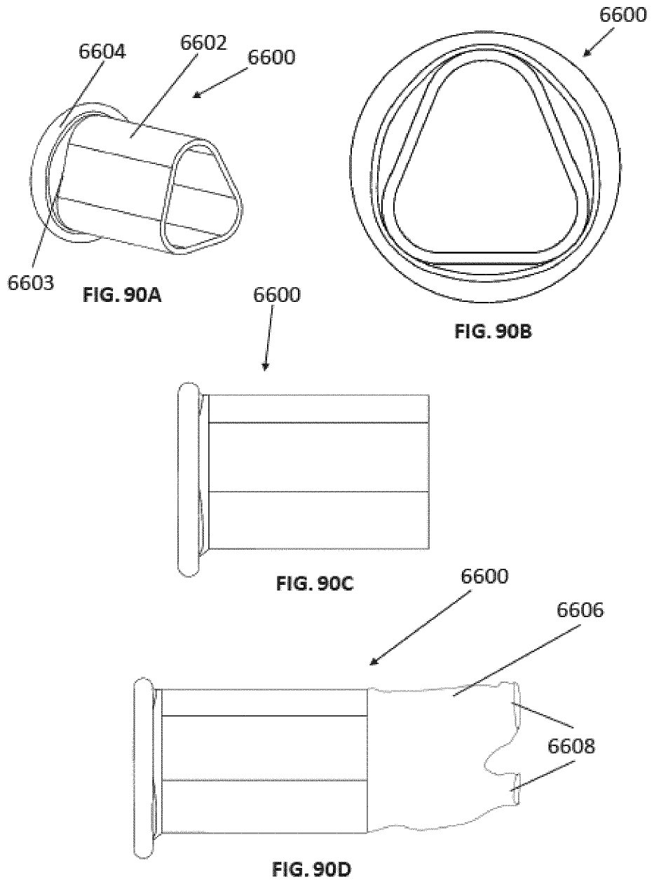

FIGS. 90A-90D illustrate another alternative component of a medical access device for use with a body opening. In particular, FIGS. 90A-90D depict an alternative embodiment of outer member 6002 of FIGS. 84A-84D, as outer member 6600. Outer member 6600 has a semi-rigid triangular shaft or tube 6602 that transitions to a ring 6604 at a distal end 6603. The outer member 6600 is designed to fold up to be inserted in an opening such as a vagina. Both the triangular shaft 6602 and ring 6604 may be made of flexible material capable of distorting and folding in any direction. Once outer member 6600 is inserted in a vagina, the outer member 6600 may spring open to its original shape and the ring 6604 may be utilized for anchoring and sealing outer member 6600 within the vagina. FIG. 90D depicts the outer member 6600 with a valved sleeve end or valved cap 6606 (similar to sleeve end or cap 6014 of FIG. 84D) attached which may allow for medical instrument access through openings 6608 in the valved sleeve 6606.

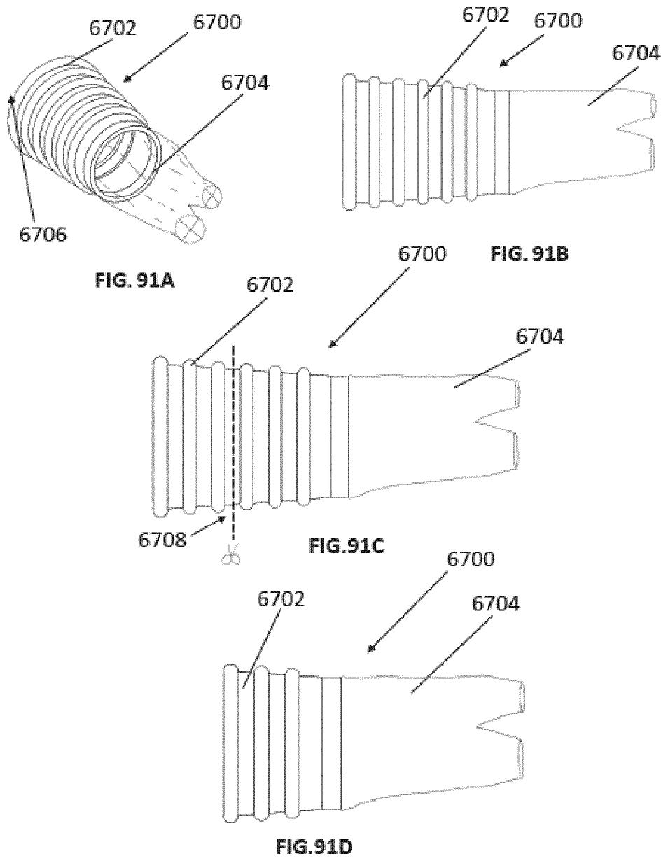

FIGS. 91A-91D illustrate a medical access device for use with a body opening, according to another example. FIGS. 91A-91D depict a medical device 6700 that is semi rigid and has a ribbed/ring anchor component 6702 that is attached to a valved sleeve 6704. Valved sleeve 6704 may have one or more openings that medical instruments may be inserted through. The ribbed anchor 6702 may have the largest diameter of rib at a distal end 6706 of the ribbed anchor 6702. The diameter of rib size may decrease on each ring starting at the distal end 6706 and moving towards the valved sleeve 6704. The most distal rib may be capable of anchoring the medical device 6800 to the vagina. The semi rigid ribbed anchor 6702 may be trimable between ribs so that, depending on a patient's anatomy, the ribbed anchor may be cut to better fit a patient's body opening (e.g. vagina). FIG. 91C shows a dashed line 6708 that depicts an example location where the ribbed anchor 6702 may be cut. FIG. 91D shows an example of a cut ribbed anchor 6702 that was cut along the dashed line 6708 from FIG. 8C.

FIGS. 84-90 demonstrate embodiments of a medical access device that may have the following attributes. When the medical access devices and medical instruments are in-situ in a vagina or an alternative orifice, the medical access device structure may maximize space above the instrument that may otherwise be pushed into the center of the opening due to the forces put on the outer medical access device by the vaginal walls. The medical access device may help maintain a medical instrument (e.g., cannula) off-center, so that the upper lumen forms a maximum (or near maximum) sized single opening for additional instruments. Further, certain embodiments, by having flexible walls or being flexible in nature, may allow for easier access of placing the medical access device into an orifice (e.g. vaginas).

FIGS. 91A-91D depict a medical access device that has the attribute of being trimmed in order to fit many different size openings based on a user's anatomy. By increasing the diameter of ribs of the semi-ribbed anchor and being capable of being trimmed between any ribs, the medical device may be utilized by many different users with different sized orifices (e.g. vaginas). Medical providers may utilize a single medical access device for patients who have various size vaginas.

It will be understood that any of the medical device used herein may include any suitable medical grade material. Any medical tool sized and shaped to access the lumens of any of the medical devices described herein may be used to access a body cavity. Unless otherwise stated, a body opening may be any natural orifice (e.g., a vagina, anus, or the like) or incision formed in the body. A body cavity may be any cavity within the body accessed via the body opening or the incision.

It will also be understood that medical tools described herein may be disassembled or unattached at various positions along their length. This may allow a user to insert medical tools into a body opening before a medical device is inserted. Subsequently, the medical device may be placed over a portion of the medical tool and advanced toward the opening. The medical device may then be positioned in the body opening in any manner described herein. Once the medical device is positioned, the medical tool may be reassembled or reattached to those portions from which it was unattached.

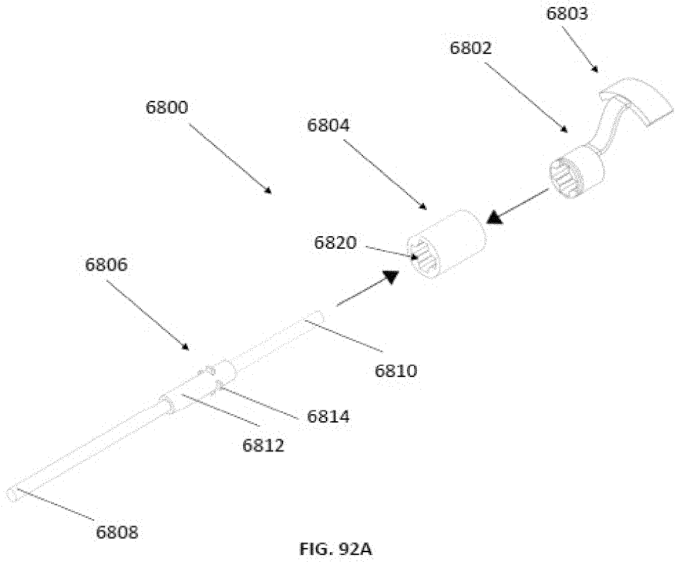

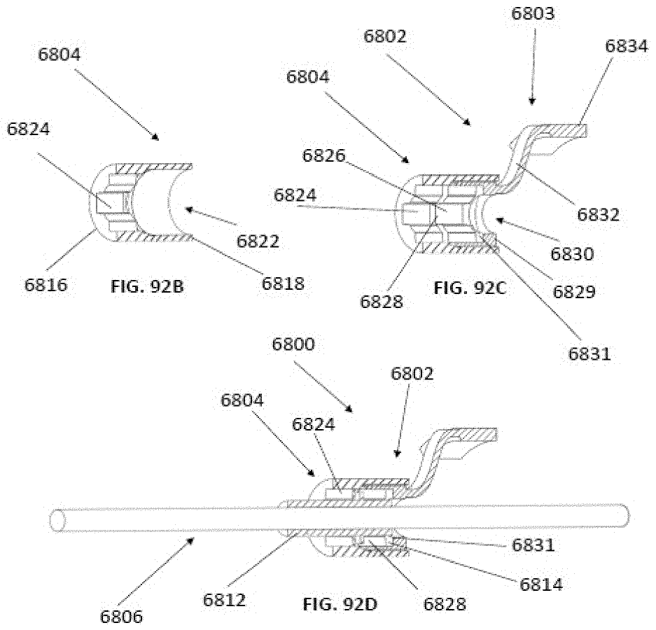

FIGS. 92A-92D illustrate a medical access device for use with a body opening, according to another example. FIG. 92A depicts a medical device 6800 that may include a base 6802, a housing 6804, and a shaft 6806. Shaft 6806 may be a straight shafted uterine manipulator. Shaft 6806 may extend from a proximal end 6808 to a distal end 6810. Shaft 6806 may be cylindrical in nature. Shaft 6806 may include a segment 6812 that wraps around a section of shaft 6806 and is located between the proximal end 6808 and distal end 6810. Segment 6812 may include one or more teeth 6814. In one embodiment, segment 6812 may have four teeth 6814. Teeth 6814 may protrude perpendicular to segment 6812. The teeth 6814 may all be located 90 degrees apart from one another along the radial exterior of segment 6812. Teeth 6814 may be capable of being received by slots 6824 of housing 6804 and slots 6826 of base 6802 (FIGS. 92B and 92C). The teeth may further be used to help rotate cup 6803 as will be described in greater detail below.

FIG. 92B depicts housing 6804 from FIG. 92A. Housing 6804 may have a proximal end 6816 and a distal end 6818. Proximal end 6816 may have an opening 6820 that extend through housing 6804 and to distal end 6818. Opening 6820 may be capable of receiving a distal end 6810 of shaft 6806. Opening 6820 may be in the shape of the distal end of shaft 6806 and further include space for segment 6812 and teeth 6814. The opening 6820 may have four slots 6824 that may receive teeth 6814. The distal end 6818 of housing 6804 may have an enlarged opening 6822 that is capable of receiving a proximal end 6828 of base 6802. The diameter of enlarged opening 6822 may be greater than the diameter of opening 6820. The four slots 6824 may stop extending once opening 6820 meets enlarged opening 6822.

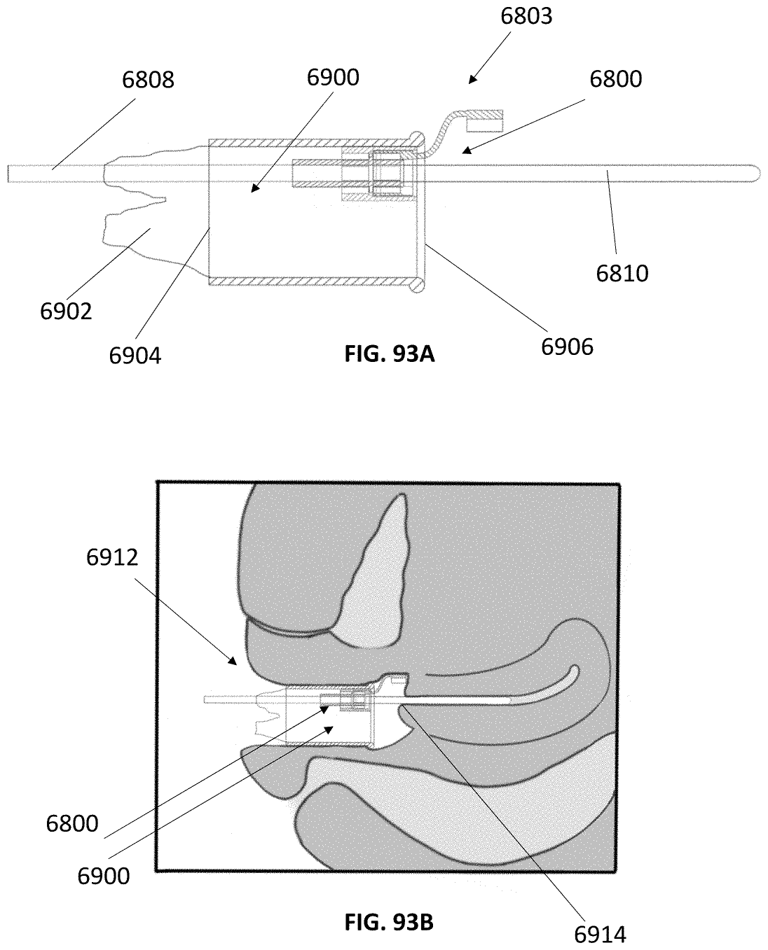

FIG. 92C depicts housing 6804 connected to the base 6802. Base 6802 may have a proximal end 6828 that may be received by opening 6822 of housing 6804. Proximal end 6828 may be rounded. Base 6802 may have an opening 6830 that extends from proximal end 6828 to the distal end 6829 of base 6802. The opening 6830 may be a similar shape to opening 6820. Opening 6830 may be in the shape of the distal end 6810 of shaft 6806 and further include space for segment 6812 and teeth 6814. Opening 6830 may narrow and no longer have space for teeth 6814 close to a distal end of 6829 at narrow opening 6831. Narrow opening 6831 may be located at the base of the cup 6803. Narrow opening 6831 may be the circumference of segment 6812, but no longer protrude outward at four locations (e.g., slots 6826) that correspond to the protrusion of teeth 6814. The opening 6830 may have four slots 6826 that may receive teeth 6814. Base 6802 may include a portion of a cup 6803 at a distal end 6829. Base 6802 may be a colpotomy cup. Base 6802 may be able to rotate freely within housing 6804 as discussed in greater detail below. The portion of a cup 6803 may be a half-cup or a portion of a fully cylindrical cup. The cup 6803 may be a half-cup or less to reduce the bulk of the culpotomy cup in the space near the cervix in order to provide more room for instruments when medical device 6800 is inserted in a body opening 6912 (shown in FIG. 93B). This may take place during a colpotomy. The cup 6803 may be capable of rotating to reach all 360 degrees during the a medical procedure such as a colpotomy. The portion of a cup 6803 may include an arm 6832 and curved wall 6834. Arm 6832 may start at the distal end 6829 at base 6802 and protrude outwards from the distal end 6829. The arm 6832 may end at curved wall 6834. Curved wall 6834 may be the wall of a colpotomy cup.

FIG. 92D depicts the medical device 6800 when the base 6802, a housing 6804, and a shaft 6806 are connected. In order for the medical device 6800 to be connected, the distal end 6810 of the shaft 6806 may be inserted into the opening 6820 of proximal end 6816 of the housing 6804. Further, the distal end 6810 of the shaft 6806 may be inserted into opening 6830 of proximal end 6828 of base 6802. In addition to the shaft 6806 being inserted in the base 6802 and housing 6804, the proximal end 6828 of the base 6802 may be received by the opening 6822 of the housing 6804 so that the housing 6804 may receiving the base 6802. The slots 6824 of housing 6804 and slots 6826 of base 6802 may align when the housing 6804 receives the base 6802. The teeth 6814 may contact the inside of base 6802 at narrow opening 6831. Medical device 6800 may employ the idea of a key and keyhole mechanism to achieve rotation of the built in cup 6803 using shaft 6806. This may be done by allowing for teeth 6814 of shaft 6806, to contact and rotate cup 6803 at the start of narrow opening 6831, where slots 6826 stops extending.

Once medical device 6800 is connected (e.g. the shaft 6806, housing 6804, and base 6802 are connected as described in FIG. 92D), rotating the shaft 6806 may cause the teeth to contact the slots 6828 of base 6802 and cause the base 6802 and the cup 6803 to rotate. In on embodiment, the base 6802 and cup 6803 may be capable of rotating 360 degrees in either direction. Base 6802 may be capable of rotating while housing 6804 does not rotate.

FIGS. 93A-93B illustrate a medical access device for use with a body opening, according to another example. FIG. 93A may depicts medical device 6800 attached to a conduit 6900. The conduit may be any access device described in the application herein such as outer access member 6002 (described in FIG. 84A-91D)The conduit may have a proximal end 6904 and a distal end 6906. The conduit 6900 may have one or more valves 6902 that are capable of receiving instruments and located at a proximal end 6904 of conduit 6900. When the medical device 6800 is connected to conduit 6900, the proximal end 6808 of shaft 6806 may extend externally of a valve 6902. Medical device 6800 may be held in place in conduit 6900 by any of the sealing techniques discussed herein. For instance, conduit 6900 may include a flexible membrane 105 that may be capable of inflation. When the medical device 6800 is connected to conduit 6900, the distal end 6810 of shaft 6806 may extend externally of the distal end 6906 of conduit 6900. Further, the portion of the cup 6803 may extend externally of the distal end 6906 of conduit 6900.

FIG. 93B may depicts the medical device 6800 attached to conduit 6900 and located within an opening 6912. The opening 6912 may be a vagina. The medical device 6800 may be located in situ within the vagina. The conduit 6900 and valves 6902 may allow for pressure, such as pneumovagina/pneumoperitoneum pressure, within the vagina and/or abdomen to be maintained during a procedure (e.g., a copotomy). The distal end 6810 of shaft 6806 may be capable of entering the cervix 6914 of an individual during a medical procedure.

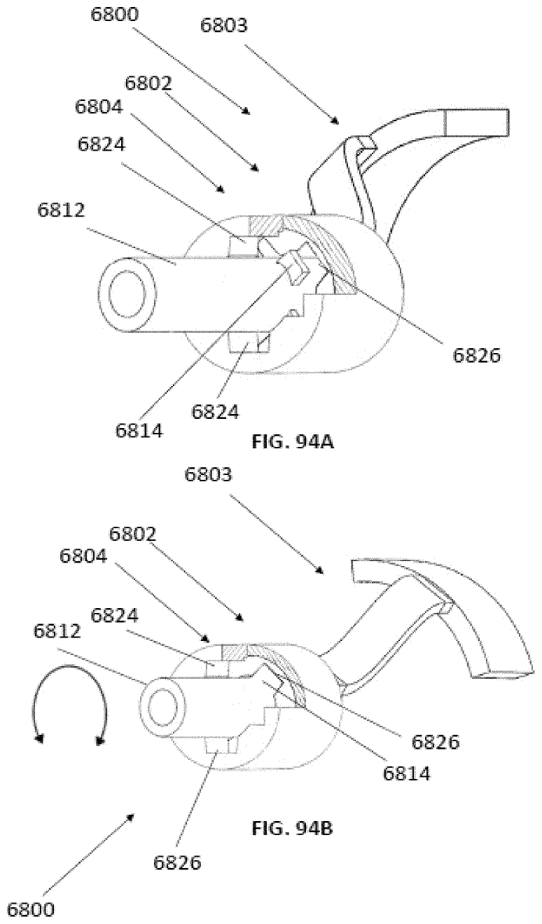

FIGS. 94A-94B illustrate a medical access device for use with a body opening, according to another example. FIG. 94A and 94B may depict segment 6812 while connecting to housing 6804 and base 6802. FIG. 94A depicts segment 6812 immediately before the teeth 6814 contact the inner base 6802 at narrow opening 6831. FIG. 94B depicts segment 6812 once the teeth 6814 contact the inner base 6802 at narrow opening 6831. FIG. 94B thus depicts medical device 6800 attached (e.g. the base 6802, housing 6804, and shaft 6806 are attached). The medical device 6800 may be considered attached once teeth 6814 move through corresponding slots 6824 of housing 6804 and through slots 6826 of base 6802 to contact the inner base of base 6802 where the narrow opening 6831 is located. Once teeth 6814 contact the inner base 6802 at narrow opening 6831 at the base of 6803, the shaft 6806 may rotate in either direction, allowing for the cup 6803 to rotate independent of the housing 6804. This may allow for slots 6824 of housing 6804 to not align with the slots 6826 of base 6802.

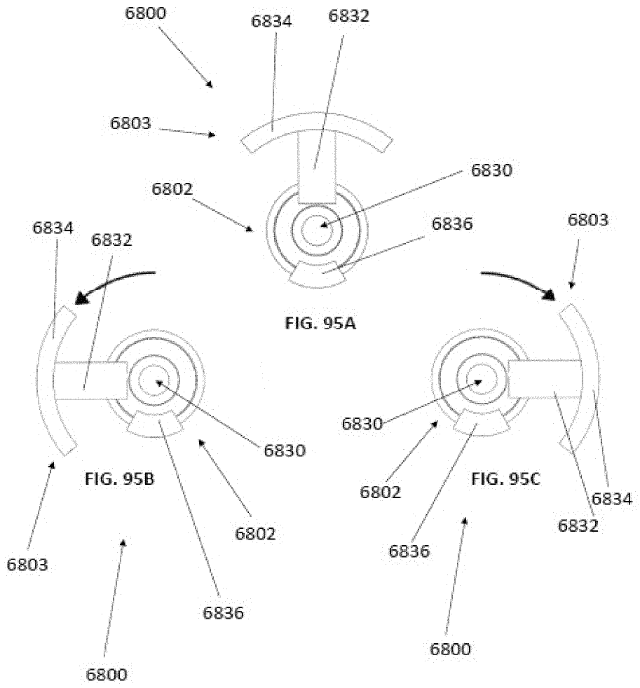

FIGS. 95A-94C illustrate a medical access device for use with a body opening, according to another example. FIG.94A-94C may depict the medical device 6800 once the shaft, 6806, housing 6804, and base 6802 are connected. FIG.94A-94C may depict an embodiment of medical device 6800 that has a stopper 6836 that may prevent the cup 6803 from rotating a full 360 degrees. Further, the stopper 6836 may prevent the base of the cup 6803 from being pushed out of housing 6804. FIG. 95A may depict the medical device 6800 prior to any rotation of base 6802 and cup 6803. FIG. 95B depicts the medical device 6800 and more specifically cup 6803, rotating counter-clockwise. FIG. 95C depicts the medical device 6800 ad more specifically cup 6803 rotating clockwise. In both FIGS. 95B and 95C, the cup 6803 is restricted from rotating 360 degrees in either direction due to stopper 6836. In one embodiment, the base 6802 and cup 6803 may only rotate until the cup 6803 (e.g., the arm 6832) contacts the stopper 6836.

It will be apparent to those skilled in the art that various modifications and variations can be made to the disclosed device without departing from the scope of the disclosure. For example, the configurations of valves and/or detachable containers on the cap may change in size, number, and shape based on the size of the opening, the type of procedure to be performed, and other factors. In other examples, the proximal ring, the distal ring, the sleeve, and any middle rings or other structures discussed herein may be used in combination. For example, the type of sleeve may be changed based on the required rigidity to retract an opening wall.

While use of the device discussed herein has been limited to vaginal procedures, the device may be used to access any bodily cavity in humans or animals. Various components of the medical access device may be changed based on the size and/or location of the body opening, and/or the medical procedure to be performed via the body opening. Further, embodiments allow different auxiliary medical devices to access the body via the body opening, while protecting tissue of the body opening and/or the auxiliary medical devices at least at the body opening. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A medical device configured to protect a body opening, the medical device comprising:
a sleeve having a proximal end and a distal end;
a proximal ring attached to the proximal end of the sleeve; and
a distal ring attached to the distal end of the sleeve.

2. The device of claim 1, wherein the sleeve is a double sleeve with an inner wall and an outer wall.

3. The device of claim 2, wherein the inner wall is configured to create an inner seal when the sleeve is inflated.

4. The device of claim 3, wherein the outer wall is configured to retract a wall of a body cavity and create an outer seal with the wall of the body cavity when the sleeve is inflated.

5. The device of claim 4, wherein the inner seal is configured to seal around a surgical tool inserted through the proximal ring and the distal ring.

6. The device of claim 2, further comprising a cap attached to a proximal side of the proximal ring, wherein the cap seals around the proximal ring and comprises one or more valves.

7. A medical device configured to protect a body opening, the medical device comprising:
a sleeve having a proximal end and a distal end;
a proximal ring attached to the proximal end of the sleeve;
a distal ring attached to the distal end of the sleeve; and
one or more valves at the proximal end of the sleeve.

8. The medical device of claim 7, further comprising:
a cap attached to a proximal side of the proximal ring, wherein the cap seals
around the proximal ring and the one or more valves are mounted on the cap;

9. The medical device of claim 8, wherein the cap further comprises a detachable container.

10. The medical device of claim 7, wherein the sleeve further comprises a plurality of middle rings.

11. The medical device of claim 10, wherein each of the plurality of middle rings has a different middle ring diameter;
wherein the proximal ring diameter is smaller than smallest middle ring diameter;
wherein the middle ring diameter of each of the plurality of middle rings increases as the plurality of middle rings approach the distal ring; and
wherein the distal ring diameter is larger than the largest middle ring diameter.

12. The medical device of claim 11, wherein the plurality of middle rings are inflatable.

13. The medical device of claim 7, further comprising a cap attached to a proximal side of the proximal ring , wherein the cap comprises of one or more insufflation ports and wherein the insufflation ports may be used to inflate the sleeve and the distal ring.

14. A method of using a medical device configured to protect a body to perform a surgical procedure, the method comprising:
positioning the medical device within a body cavity, the medical device comprising:
a sleeve having a proximal end, a distal end, an outer wall, and an inner wall;
a proximal ring attached to the proximal end of the sleeve;
a distal ring attached to the distal end of the sleeve; and
wherein the proximal ring is positioned outside the body cavity and the distal ring is positioned inside the body cavity;
expanding the outer wall of the sleeve so that a cavity wall of the body cavity retracts;
inserting one or more surgical tools through the medical device and into the body cavity; and
performing the surgical procedure using at least the one or more surgical tools.

15. The method of claim 14, wherein the inner wall seals around the one or more surgical tools and the outer wall seals with the cavity wall.

16. The method of claim 14, wherein the medical device further comprises a cap attached to the proximal ring and wherein the cap has one or more valves configured to receive and seal around surgical tools and one or more detachable containers.

17. The method of claim 16, further comprising:
removing tissue from the cavity using the one or more surgical tools;
placing the removed tissue into the detachable container; and
detaching the detachable container from the cap.

18. The method of claim 17, wherein the surgical tools are one of a scalpel, a grasper, an imaging device, a cutting device, or a suction device.

19. The method of claim 14, wherein expanding the outer wall is done by inflating the sleeve.

20-49. (canceled)
